(19) United States
(12) Patent Application Publication
(10) Pub. No.: US 2016/0222460 A1
(43) Pub. Date: Aug. 4, 2016

(54) MOLECULAR DIAGNOSTIC TEST FOR OESOPHAGEAL CANCER

(71) Applicant: ALMAC DIAGNOSTICS LIMITED, Craigavon, Armagh (GB)

(72) Inventors: Karen KEATING, Magherafelt (GB); Laura HILL, Lisburn (GB); Steve DEHARO, Lisburn (GB); Eamonn O'BRIEN, Hillsborough (GB); Tim DAVISON, Hillsborough (GB); Paul HARKIN, Dromore (GB); Richard KENNEDY, Belfast (GB); Jude O'DONNELL, Galbally (GB)

(21) Appl. No.: 14/917,925

(22) PCT Filed: Sep. 9, 2014
(86) PCT No.: PCT/GB2014/052727

§ 371 Date: Mar. 9, 2016

(30) Foreign Application Priority Data
Sep. 9, 2013 (GB) 1316027.0

(51) Int. Cl. C12Q 1/68

(52) U.S. Cl. CPC C12Q 1/6886; C12Q 2600/106; C12Q 2600/158

(57) ABSTRACT

Methods and compositions are provided for the identification of a molecular diagnostic test for oesophageal adenocarcinoma (OAC). The test defines a novel DNA damage repair deficient molecular subtype and enables classification of a patient within this subtype. The present invention can be used to determine whether patients with OAC are clinically responsive or non-responsive to a therapeutic regimen prior to administration of any chemotherapy. This test may be used with different drugs that directly or indirectly affect DNA damage or repair, such as many of the standard cytotoxic chemotherapeutic drugs currently in use. In particular, the present invention is directed to the use of certain combinations of predictive markers, wherein the expression of the predictive markers correlates with responsiveness or non-responsiveness to a therapeutic regimen.

### FIELD OF THE INVENTION

The present invention relates to a molecular diagnostic test useful for predicting responsiveness of oesophageal cancers to particular treatments that includes the use of a DNA damage repair deficiency subtype. The invention includes the generation and use of various classifiers derived from identification of this subtype in oesophageal cancer patients (in particular oesophageal adenocarcinoma (OAC) patients), such as use of a 44-gene classification model that is used to identify this DNA damage repair deficiency molecular subtype. One application is the stratification of response to, and selection of patients for Oesophageal cancer therapeutic drug classes, including DNA damage causing agents and DNA repair targeted therapies. The present invention provides a test that can guide conventional therapy selection as well as selecting patient groups for enrichment strategies during clinical trial evaluation of novel therapeutics. DNA repair deficient subtypes can be identified, for example, from fresh/frozen (FF) or formalin fixed paraffin embedded (FFPE) patient samples.

### BACKGROUND

The pharmaceutical industry continuously pursues new drug treatment options that are more effective, more specific or have fewer adverse side effects than currently administered drugs. Drug therapy alternatives are constantly being developed because genetic variability within the human population results in substantial differences in the effectiveness of many drugs. Therefore, although a wide variety of drug therapy options are currently available; more therapies are always needed in the event that a patient fails to respond.

Traditionally, the treatment paradigm used by physicians has been to prescribe a first-line drug therapy that results in the highest success rate possible for treating a disease. Alternative drug therapies are then prescribed if the first is ineffective. This paradigm is clearly not the best treatment method for certain diseases. For example, in diseases such as cancer, the first treatment is often the most important and offers the best opportunity for successful therapy, so there exists a heightened need to choose an initial drug that will be the most effective against that particular patient's disease.

Oesophageal cancer (cancer of the food pipe) is a highly aggressive disease, ranking among the 10 most common cancers in the world. It has become more common in the past 30 years in the UK. It is the 9th most common cancer in adults, with around 8,500 cases diagnosed each year in the UK (CRUK statistics). Oesophageal cancer is about twice as common in men as in women. Adenocarcinoma means a cancer that has started in glandular cells. In oesophageal cancer, these are the cells that make mucus in the lining of the oesophagus. The number of adenocarcinomas has increased in the last 20 years. They now make up just over a half of all oesophageal cancers diagnosed, with Squamous cell carcinoma accounting for the remaining occurrences. Adenocarcinomas are found mainly in the lower third of the oesophagus.

Treatment of early stage Oesophageal adenocarcinoma (OAC) is generally surgery. Surgical therapy is the only treatment that has repeatedly been shown to provide prolonged survival, albeit in only approximately 20% of cases (Müller J M et al. Br J Surg (1990) 77:845). The results of surgical resection can be excellent. Five year survival rate is over 80% when tumours are confined to the mucosa and between 50% and 80% when the submucosa is involved. Bonavina L. Br J Surg (1995) 82:98; Hölscher A H, et al. Br J Surg (1997) 84:1470.

The advent of microarrays and molecular genomics has the potential for a significant impact on the diagnostic capability and prognostic classification of disease, which may aid in the prediction of the response of an individual patient to a defined therapeutic regimen. Microarrays provide for the analysis of large amounts of genetic information, thereby providing a genetic fingerprint of an individual. There is much enthusiasm that this technology will ultimately provide the necessary tools for custom-made drug treatment regimens.

Currently, healthcare professionals have few mechanisms to help them identify cancer patients who will benefit from chemotherapeutic agents. Identification of the optimal first-line drug has been difficult because methods are not available for accurately predicting which drug treatment would be the most effective for a particular cancer's physiology. This deficiency results in relatively poor single agent response rates and increased cancer morbidity and death. Furthermore, patients often needlessly undergo ineffective, toxic drug therapy.

Molecular markers have been used to select appropriate treatments, for example, in breast cancer. Breast tumors that do not express the estrogen and progesterone hormone receptors as well as the HER2 growth factor receptor, called “triple negative”, appear to be responsive to PARP-1 inhibitor therapy (Linn, S. C., and Van't Veer, L., J. Eur J Cancer 45 Suppl 1, 11-26 (2009); O'Shaughnessy, J., et al. N Engl J Med 364, 205-214 (2011). Recent studies indicate that the triple negative status of a breast tumor may indicate responsiveness to combination therapy including PARP-1 inhibitors, but may not be sufficient to indicate responsiveness to individual PARP-1 inhibitors (O'Shaughnessy et al., 2011).

Furthermore, there have been other studies that have attempted to identify gene classifiers associated with molecular subtypes to indicate responsiveness of chemotherapeutic agents (Farmer et al. Nat Med 15, 68-74 (2009); Konstantinopoulos, P. A., et al., J Clin Oncol 28, 3555-3561 (2010)).

WO 2012/037378 describes a 44-gene DNA microarray assay, the DNA damage repair deficient (DDRD) assay. This assay identifies a molecular subgroup of cancers that have lost the DNA damage response FA/BRCA pathway, resulting in sensitivity to DNA damaging chemotherapeutic agents (Kennedy & D'Andrea Journal of Clinical Oncology (2006) 24:3799, Turner et al Nature Reviews Cancer (2004) 4:814).

In breast cancer the DDRD assay has been shown to predict response to neoadjuvant DNA-damaging chemotherapy (5-fluorouracil, anthracycline and cyclophosphamide) in 203 breast cancer patients (odd ratio 4.01) (95% CI:1.69-9.54). In a cohort of 191 early breast cancer patients treated with adjuvant 5-fluorouracil, epirubicin and cyclophosphamide treatment, the assay predicted 5-year relapse free survival with a hazard ratio of 0.37 (95% CI:0.15-0.88).

### SUMMARY OF THE INVENTION

In many instances of OAC, surgery follows neoadjuvant chemotherapy, usually a combination of cisplatin and fluorouracil. This therapy is used to reduce the size of the tumour. However it is known that not all patients respond to this form of therapy and there is, as yet, no test to identify those OAC patients that will benefit from neoadjuvant chemotherapy. Consequently many patients are undergoing chemotherapy, and the associated side effects and reduction in quality of life, without a therapeutic benefit. The DNA damage response pathway has been shown to be important in the progression of oesophageal cancer (He et al (2013) Carginogeneisis 34:139; Motoori et al International Journal of Oncology (2010) 37:1113). Consequently there is a great clinical need for a test to stratify OAC patients with respect to neoadjuvant chemotherapy in advance of surgery.

The present invention is based upon application of methods that identify deficiencies in DNA damage repair to determine which patients will benefit from certain therapies, such as platinum-based therapy in order to treat oesophageal cancer, specifically OAC. The invention is directed to methods of using a collection of gene product markers expressed in oesophageal cancer such that when some or all of the transcripts are over or under-expressed, they identify a subtype of oesophageal cancer that has a deficiency in DNA damage repair. The invention also provides methods for indicating responsiveness or resistance to DNA-damaging therapeutic agents. In different aspects, this gene or gene product list may form the basis of a single parameter or a multiparametric predictive test that could be delivered using methods known in the art such as microarray, Q-PCR, immunohistochemistry, ELISA or other technologies that can quantify mRNA or protein expression.

Thus, according to one aspect of the invention there is provided a method of predicting responsiveness of an individual having oesophageal cancer such as (in particular) oesophageal adenocarcinoma (OAC) to treatment with a DNA-damaging therapeutic agent comprising:

a. measuring expression levels of one or more biomarkers/genes in a test sample obtained from the individual, wherein the one or more biomarkers are selected from Table 1A, 1B, 2A, 2B, 3 and/or 4 such as one or more biomarkers/genes selected from the group consisting of CXCL10, MX1, IDO1, IF144L, CD2, GBP5, PRAME, ITGAL, LRP4, and APOL3;

b. deriving a test score that captures the expression levels;

c. providing a threshold score comprising information correlating the test score and responsiveness;

d. and comparing the test score to the threshold score; wherein responsiveness is predicted when the test score exceeds the threshold score and/or wherein a lack of responsiveness is predicted when the test score does not exceed the threshold score.

The methods may be performed as a method for selecting a suitable treatment for an individual or as a test and treat method. Thus, in certain embodiments if the test score exceeds the threshold score (responsiveness is predicted) the individual is treated with the DNA-damaging therapeutic agent. Similarly, if the test score does not exceed the threshold score (responsiveness is not predicted) the individual is not treated with the DNA-damaging therapeutic agent. In those circumstances, alternative treatments may be contemplated. For OAC, the alternative treatments may comprise administration of a mitotic inhibitor, such as a vinca alkaloid or a taxane. Example vinca alkaloids include vinorelbine. Example taxanes include paclitaxel or docetaxel. Alternatively, the treatment may exclude chemotherapy altogether. The methods can, in some embodiments, also involve the subsequent treatment of the individual identified as responsive. Corresponding kits are also contemplated. The method is typically performed in vitro. The method is, therefore, performed using an isolated, or pre-isolated, sample. In some embodiments, the methods may encompass the step of obtaining a test sample from the individual. In certain embodiments, the method comprises measuring an expression level of at least 10 of the biomarkers from any of Tables 1 to 4 in the test sample. More specifically, the method may comprise measuring the expression level of at least 10 and up to all 44 different biomarkers listed in Table 2B in some embodiments. In certain embodiments, expression levels are measured using primers or probes which bind to at least one of the target sequences set forth in Tables 1A, 1B or 3 (as SEQ ID NO: 1-24, SEQ ID NO: 25-50 or SEQ ID NO: 51-230).

In some embodiments, the method further comprises measuring an expression level of one or more biomarkers in the test sample, wherein the one or more biomarkers are selected from the group consisting of CDR1, FYB, TSPAN7, RAC2, KLHDC7B, GRB14, AC138128.1, KIF26A, CD274, CD109, ETV7, MFAP5, OLFM4, PI15, FOSB, FAM19A5, NLRC5, PRICKLE1, EGR1, CLDN10, ADAMTS4, SP140L, ANXA1, RSAD2, ESR1, IKZF3, OR2I1P, EGFR, NAT1, LATS2, CYP2B6, PTPRC, PPP1R1A, and AL137218.1. In certain embodiments, the test score captures the expression levels of all of the biomarkers. In some embodiments, responsiveness may be predicted when the test score exceeds a threshold score at a value of between approximately 0.1 and 0.5 such as 0.1, 0.2, 0.3, 0.4 or 0.5. for example approximately 0.3681.

The oesophageal cancer is typically oesophageal adenocarcinoma (OAC) and may be early stage. Alternatively, the OAC may be late stage or metastatic disease. The treatment for which responsiveness is predicted is typically neoadjuvant treatment. However, it may comprise adjuvant treatment additionally or alternatively.

The invention described herein is not limited to any one DNA-damaging therapeutic agent; it can be used to identify responders and non-responders to any of a range of DNA-damaging therapeutic agents, for example those that directly or indirectly affect DNA damage and/or DNA damage repair. In some embodiments, the DNA-damaging therapeutic agent comprises one or more substances selected from the group consisting of: a DNA damaging agent, a DNA repair targeted therapy, an inhibitor of DNA damage signalling, an inhibitor of DNA damage induced cell cycle arrest, a histone deacetylase inhibitor, a heat shock protein inhibitor and an inhibitor of DNA synthesis. More specifically, the DNA-damaging therapeutic agent may be selected from one or more of a platinum-containing agent, a nucleoside analogue such as gemcitabine or 5-fluorouracil or a prodrug thereof such as capecitabine, an anthracycline such as epirubicin or doxorubicin, an alkylating agent such as cyclophosphamide, an ionising radiation or a combination of radiation and chemotherapy (chemoradiation). In particular embodiments, the DNA-damaging therapeutic agent comprises a platinum-containing agent, such as a platinum based agent selected from cisplatin, carboplatin and oxaliplatin. For example, it is shown experimentally herein that the methods of the invention can predict responsiveness/act as a prognostic indicator of OAC patients who are more likely to benefit from platinum based therapies. The methods may predict responsiveness to treatment with the DNA-damaging therapeutic agent together with a further therapy or drug. Thus, the methods may predict responsiveness to a combination therapy. Thus, in some embodiments, the further drug is a mitotic inhibitor. The mitotic inhibitor may be a vinca alkaloid or a taxane. In specific embodiments, the vinca alkaloid is vinorelbine. In other embodiments, the taxane is paclitaxel or docetaxel. In certain embodiments, responders to the following (combination) treatments are identified: cisplatin/carboplatin and 5-fluorouracil (CF), cisplatin/carboplatin and capecitabine (CX), epirubicin/doxorubicin, cisplatin/carboplatin and fluorouracil (ECF), epirubicin/doxorubicin, oxaliplatin and capecitabine (EOX), and combinations with paclitaxel, irinotecan and vinorelbine, radiation or chemoradiation with or without taxanes, in treatment of oespohageal cancer.

The present invention relates to prediction of response to drugs (DNA-damaging therapeutic agents) using different classifications of response, such as overall survival, progression free survival, disease free survival, radiological response, as defined by RECIST, complete response, partial response, stable disease and serological markers such as, but not limited to, PSA, CEA, CA125, CA15-3 and CA19-9. In other embodiments of this invention it can be used to evaluate endoscopic ultrasound, CT, spiral CT, FDG-PET, pathologic, histological response in oesophageal cancer (OAC) treated with DNA damaging combination therapies, alone or in the context of standard treatment.

The present invention relies upon a DNA damage response deficiency (DDRD) molecular subtype, originally identified in breast and ovarian cancer (WO2012/037378; incorporated herein by reference). This molecular subtype can, in some embodiments, be detected by the use of two different gene classifiers—one being 40 genes in length and one being 44 genes in length. The DDRD classifier was first defined by a classifier consisting of 53 probesets on the Almac Breast Disease Specific Array (DSA™). So as to validate the functional relevance of this classifier in the context of its ability to predict response to DNA-damaging containing chemotherapy regimens, the classifier needed to be re-defined at a gene level. This facilitated evaluation of the DDRD classifier using microarray data from independent datasets that were profiled on microarray platforms other than the Almac Breast DSA®. In order to facilitate defining the classifier at a gene level, the genes to which the Almac Breast DSA® probesets map needed to be defined. This involved the utilization of publicly available genome browser databases such as Ensembl and NCBI Reference Sequence. The 44-gene DDRD classifier model supersedes that of the 40-gene DDRD classifier model. The results presented herein demonstrate that the 44 and 40 gene classifier models and related classifier models derived from the markers disclosed herein are effective and significant predictors of response to chemotherapy regimens that contain DNA damaging therapeutics in the context of OAC.

The identification of the DDRD subtype using classifier models based upon genes taken from Tables 1A and 1B (or Tables 3 and 4), such as by both the 40-gene classifier model (see Tables 2A and 2B) and the 44-gene classifier model, can be used to predict response to, and select patients for, standard OAC therapeutic drug classes, including DNA damage causing agents and DNA repair targeted therapies.

In another aspect, the present invention relates to kits for conventional diagnostic uses listed above such as nucleic acid amplification, including PCR and all variants thereof such as real-time and end point methods and qPCR, Next generation Sequencing (NGS), microarray, and immunoassays such as immunohistochemistry, ELISA, Western blot and the like. Such kits include appropriate reagents and directions to assay the expression of the genes or gene products and quantify mRNA or protein expression. The kits may include suitable primers and/or probes to detect the expression levels of at least one of the genes in Table 1A and/or 1B and/or 2A and/or 2B and/or 3 and/or 4. The kits may contain primers and/or probes that bind to target sequences set forth in Tables 1A, 1B and/or 3. The target sequences may comprise, consist essentially of or consisting of SEQ ID NO: 1-24, SEQ ID NO: 25-50 or SEQ ID NO: 51-230. Where expression is determined at the protein level the kit may contain binding reagents specific for the proteins of interest. The binding reagents may comprise antibodies to include all fragments and derivatives thereof. In the context of the various embodiments of the present invention the term “antibody” includes all immunoglobulins or immunoglobulin-like molecules with specific binding affinity for the relevant protein (including by way of example and without limitation, IgA, IgD, IgE, IgG and IgM, combinations thereof, and similar molecules produced during an immune response in any vertebrate, for example, in mammals such as humans, goats, rabbits and mice). Specific immunoglobulins useful in the various embodiments of the invention include IgG isotypes. The antibodies useful in the various embodiments of the invention may be monoclonal or polyclonal in origin, but are typically monoclonal antibodies. Antibodies may be human antibodies, non-human antibodies, or humanized versions of non-human antibodies, or chimeric antibodies. Various techniques for antibody humanization are well established and any suitable technique may be employed. The term “antibody” also refers to a polypeptide ligand comprising at least a light chain or heavy chain immunoglobulin variable region which specifically recognizes and binds an epitope of an antigen, and it extends to all antibody derivatives and fragments that retain the ability to specifically bind to the relevant protein. These derivatives and fragments may include Fab fragments, F(ab′)₂ fragments, Fv fragments, single chain antibodies, single domain antibodies, Fc fragments etc. The term antibody encompasses antibodies comprised of both heavy and light chains, but also heavy chain (only) antibodies (which may be derived from various species of cartilaginous fish or camelids). In specific embodiments, the antibodies may be engineered so as to be specific for more than protein, for example bi-specific to permit binding to two different target proteins as identified herein (see Tables 1 to 4).

In some embodiments, the kits may also contain the specific DNA-damaging therapeutic agent to be administered in the event that the test predicts responsiveness. This agent may be provided in a form, such as a dosage form, that is tailored to OAC treatment specifically. The kit may be provided with suitable instructions for administration according to OAC treatment regimens.

The invention also provides methods for identifying DNA damage response-deficient (DDRD) human OAC tumors. It is likely that this invention can be used to identify patients that are sensitive to and respond, or are resistant to and do not respond, to DNA-damaging therapeutic agents, such as drugs that damage DNA directly, damage DNA indirectly or inhibit normal DNA damage signaling and/or repair processes.

The invention also relates to guiding conventional treatment of patients. The invention also relates to selecting patients for clinical trials where novel DNA-damaging therapeutic agents, such as drugs of the classes that directly or indirectly affect DNA damage and/or DNA damage repair are to be tested specifically for treatment of OAC.

The present invention and methods accommodate the use of archived formalin fixed paraffin-embedded (FFPE) biopsy material, including fine needle aspiration (FNA) as well as fresh/frozen (FF) tissue, for assay of all transcripts in the invention, and are therefore compatible with the most widely available type of biopsy material. The expression level may be determined using RNA obtained from FFPE tissue, fresh frozen tissue or fresh tissue that has been stored in solutions such as RNAlater®.

### DETAILED DESCRIPTION OF THE INVENTION

Unless defined otherwise, technical and scientific terms used herein have the same meaning as commonly understood by one of ordinary skill in the art to which this invention belongs. Although any methods, devices, and materials similar or equivalent to those described herein can be used in the practice or testing of the invention, the preferred methods, devices and materials are now described.

All publications, published patent documents, and patent applications cited in this application are indicative of the level of skill in the art(s) to which the application pertains. All publications, published patent documents, and patent applications cited herein are hereby incorporated by reference to the same extent as though each individual publication, published patent document, or patent application was specifically and individually indicated as being incorporated by reference.

The articles “a” and “an” are used herein to refer to one or to more than one (i.e., to at least one) of the grammatical object of the article. By way of example, “an element” means one element or more than one element, unless explicitly indicated to the contrary.

A major goal of current research efforts in cancer is to increase the efficacy of perioperative systemic therapy in patients by incorporating molecular parameters into clinical therapeutic decisions. Pharmacogenetics/genomics is the study of genetic/genomic factors involved in an individual's response to a foreign compound or drug. Agents or modulators which have a stimulatory or inhibitory effect on expression of a marker of the invention can be administered to individuals to treat (prophylactically or therapeutically) oesophageal cancer in a patient. It is ideal to also consider the pharmacogenomics of the individual in conjunction with such treatment. Differences in metabolism of therapeutics may possibly lead to severe toxicity or therapeutic failure by altering the relationship between dose and blood concentration of the pharmacologically active drug. Thus, understanding the pharmacogenomics of an individual permits the selection of effective agents (e.g., drugs) for prophylactic or therapeutic treatments. Such pharmacogenomics can further be used to determine appropriate dosages and therapeutic regimens. Accordingly, the level of expression of a marker of the invention in an individual can be determined to thereby select appropriate agent(s) for therapeutic or prophylactic treatment of the individual.

The invention is directed to the application of a collection of gene or gene product markers (hereinafter referred to as “biomarkers”) expressed in certain oesophageal cancer tissue for predicting responsiveness to treatment using DNA-damaging therapeutic agents. In different aspects, this biomarker list may form the basis of a single parameter or multiparametric predictive test that could be delivered using methods known in the art such as microarray, Q-PCR, NGS, immunohistochemistry, ELISA or other technologies that can quantify mRNA or protein expression.

The present invention also relates to kits and methods that are useful for prognosis following cytotoxic chemotherapy or selection of specific treatments for oesophageal cancer (particularly OAC). Methods are provided such that when some or all of the transcripts are over or under-expressed, the expression profile indicates responsiveness or resistance to DNA-damaging therapeutic agents. These kits and methods employ gene or gene product markers that are differentially expressed in tumors of patients with oesophageal adenocarcinoma (OAC). In one embodiment of the invention, the expression profiles of these biomarkers are correlated with clinical outcome (response or survival) in archival tissue samples under a statistical method or a correlation model to create a database or model correlating expression profile with responsiveness to one or more DNA-damaging therapeutic agents. The predictive model may then be used to predict the responsiveness in a patient whose responsiveness to the DNA-damaging therapeutic agent(s) is unknown. In many other embodiments, a patient population can be divided into at least two classes based on patients' clinical outcome, prognosis, or responsiveness to DNA-damaging therapeutic agents, and the biomarkers are substantially correlated with a class distinction between these classes of patients. The biological pathways described herein have been shown to be predictive of responsiveness to treatment of OAC using DNA-damaging therapeutic agents.

### Predictive Marker Panels/Expression Classifiers

A collection of biomarkers as a genetic classifier expressed in oesophageal cancer/OAC tissue is provided that is useful in determining responsiveness or resistance to therapeutic agents, such as DNA-damaging therapeutic agents, used to treat oesophageal cancer/OAC. Such a collection may be termed a “marker panel”, “expression classifier”, or “classifier”. The collection is shown in Table 1. This collection was derived from an original collection of biomarkers as shown in Tables 1A and 1B (see WO 2012/037378) which were then mapped to an OAC-relevant platform (see the Example herein). Application of a classifier such as the 44 gene classifier shown in Table 2B permits responsiveness of OAC patients to DNA-damaging therapeutic agents to be determined. The invention may involve determining expression levels of any one or more of these genes or target sequences.

The biomarkers useful in the present methods are thus identified in Tables 1 to 4. These biomarkers are identified as having predictive value to determine a patient (having OAC) response to a therapeutic agent, or lack thereof. Their expression correlates with the response to an agent, and more specifically, a DNA-damaging therapeutic agent. By examining the expression of a collection of the identified biomarkers in an oesophageal tumor, in particular an adenocarcinoma or squamous cell carcinoma, it is possible to determine which therapeutic agent or combination of agents will be most likely to reduce the growth rate of the cancer, and in some embodiments, OAC cells. By examining a collection of identified transcript gene or gene product markers, it is also possible to determine which therapeutic agent or combination of agents will be the least likely to reduce the growth rate of the cancer. By examining the expression of a collection of biomarkers, it is therefore possible to eliminate ineffective or inappropriate therapeutic agents. Importantly, in certain embodiments, these determinations can be made on a patient-by-patient basis or on an agent-by-agent basis. Thus, one can determine whether or not a particular therapeutic regimen is likely to benefit a particular patient or type of patient, and/or whether a particular regimen should be continued.

TABLE 1A
Original list of genes tested in breast cancer and mapped to OAC (via Xcel array)

| Sense genes (166) | | | | |
|---|---|---|---|---|
| | EntrezGene | Antisense of known genes (24) | | SEQ ID |
| Gene Symbol | ID | Almac Gene ID | Almac Gene symbol | NO: |
| ABCA12 | 26154 | | N/A | |
| ALDH3B2 | 222 | | N/A | |
| APOBEC3G | 60489 | | N/A | |
| APOC1 | 341 | | N/A | |
| APOL6 | 80830 | | N/A | |
| ARHGAP9 | 64333 | | N/A | |
| BAMBI | 25805 | | N/A | |
| BIK | 638 | | N/A | |
| BIRC3 | 330 | AS1_BIRC3 | Hs127799.0C7n9_at | 1 |
| BTN3A3 | 10384 | | N/A | |
| C12orf48 | 55010 | | N/A | |
| C17orf28 | 283987 | | N/A | |
| C1orf162 | 128346 | | N/A | |
| C1orf64 | 149563 | | N/A | |
| C1QA | 712 | | N/A | |
| C21orf70 | 85395 | | N/A | |
| C22orf32 | 91689 | | N/A | |
| C6orf211 | 79624 | | N/A | |
| CACNG4 | 27092 | | N/A | |
| CCDC69 | 26112 | | N/A | |
| CCL5 | 6352 | | N/A | |
| CCNB2 | 9133 | | N/A | |
| CCND1 | 595 | | N/A | |
| CCR7 | 1236 | | N/A | |
| CD163 | 9332 | | N/A | |
| CD2 | 914 | | N/A | |
| CD22 | 933 | | N/A | |
| CD24 | 100133941 | | N/A | |
| CD274 | 29126 | | N/A | |
| CD3D | 915 | | N/A | |
| CD3E | 916 | | N/A | |
| CD52 | 1043 | | N/A | |
| CD53 | 963 | | N/A | |
| CD79A | 973 | | N/A | |
| CDH1 | 999 | | N/A | |
| CDKN3 | 1033 | | N/A | |
| CECR1 | 51816 | | N/A | |
| CHEK1 | 1111 | | N/A | |
| CKMT1B | 1159 | | N/A | |
| CMPK2 | 129607 | | N/A | |
| CNTNAP2 | 26047 | | N/A | |
| COX16 | 51241 | | N/A | |
| CRIP1 | 1396 | | N/A | |
| CXCL10 | 3627 | | N/A | |
| CXCL9 | 4283 | | N/A | |
| CYBB | 1536 | | N/A | |
| CYP2B6 | 1555 | | N/A | |
| DDX58 | 23586 | | N/A | |
| DDX60L | 91351 | | N/A | |
| ERBB2 | 2064 | | N/A | |
| ETV7 | 51513 | | N/A | |
| FADS2 | 9415 | | N/A | |
| FAM26F | 441168 | | N/A | |
| FAM46C | 54855 | | N/A | |
| FASN | 2194 | | N/A | |
| FBP1 | 2203 | | N/A | |
| FBXO2 | 26232 | | N/A | |
| FKBP4 | 2288 | | N/A | |
| FLJ40330 | 645784 | | N/A | |
| FYB | 2533 | | N/A | |
| GBP1 | 2633 | | N/A | |
| GBP4 | 115361 | | N/A | |
| GBP5 | 115362 | AS1_GBP5 | BRMX.5143C1n2_at | 2 |
| GIMAP4 | 55303 | | N/A | |
| GLRX | 2745 | | N/A | |
| GLUL | 2752 | | N/A | |
| GVIN1 | 387751 | | N/A | |
| H2AFJ | 55766 | | N/A | |
| HGD | 3081 | | N/A | |
| HIST1H2BK | 85236 | | N/A | |
| HIST3H2A | 92815 | | N/A | |
| HLA-DOA | 3111 | | N/A | |
| HLA-DPB1 | 3115 | | N/A | |
| HMGB2 | 3148 | | N/A | |
| HMGB3 | 3149 | | N/A | |
| HSP90AA1 | 3320 | | N/A | |
| IDO1 | 3620 | | N/A | |
| IFI27 | 3429 | | N/A | |
| IFI44 | 10561 | | N/A | |
| IFI44L | 10964 | AS1_IF144L | BRSA.1606C1n4_at | 3 |
| IFI6 | 2537 | | N/A | |
| IFIH1 | 64135 | | N/A | |
| IGJ | 3512 | AS1_IGJ | BRIH.1231C2n2_at | 4 |
| IKZF1 | 10320 | | N/A | |
| IL10RA | 3587 | | N/A | |
| IL2RG | 3561 | | N/A | |
| IL7R | 3575 | | N/A | |
| IMPAD1 | 54928 | | N/A | |
| IQGAP3 | 128239 | AS1_IQGAP3 | BRAD.30779_s_at | 5 |
| IRF1 | 3659 | | N/A | |
| ISG15 | 9636 | | N/A | |
| ITGAL | 3683 | | N/A | |
| KIAA1467 | 57613 | | N/A | |
| KIF20A | 10112 | | N/A | |
| KITLG | 4254 | | N/A | |
| KLRK1 | 22914 | | N/A | |
| KRT19 | 3880 | | N/A | |
| LAIR1 | 3903 | | N/A | |
| LCP1 | 3936 | | N/A | |
| LOC100289702 | 100289702 | | N/A | |
| LOC100294459 | 100294459 | AS1_LOC100294459 | BRSA.396C1n2_at | 6 |
| LOC150519 | 150519 | | N/A | |
| LOC439949 | 439949 | | N/A | |
| LYZ | 4069 | | N/A | |
| MAL2 | 114569 | | N/A | |
| MGC29506 | 51237 | | N/A | |
| MIAT | 440823 | | N/A | |
| MS4A1 | 931 | | N/A | |
| MX1 | 4599 | AS1_MX1 | BRMX.2948C3n7_at | 7 |
| NAPSB | 256236 | | N/A | |
| NCKAP1L | 3071 | | N/A | |
| NEK2 | 4751 | | N/A | |
| NLRC3 | 197358 | | N/A | |
| NLRC5 | 84166 | | N/A | |
| NPNT | 255743 | | N/A | |
| NQO1 | 1728 | | N/A | |
| OAS2 | 4939 | | N/A | |
| OAS3 | 4940 | | N/A | |
| PAQR4 | 124222 | | N/A | |
| PARP14 | 54625 | | N/A | |
| PARP9 | 83666 | | N/A | |
| PIK3CG | 5294 | | N/A | |
| PIM2 | 11040 | | N/A | |
| PLEK | 5341 | | N/A | |
| POU2AF1 | 5450 | | N/A | |
| PP14571 | 100130449 | | N/A | |
| PPP2R2C | 5522 | | N/A | |
| PSMB9 | 5698 | | N/A | |
| PTPRC | 5788 | | N/A | |
| RAC2 | 5880 | | N/A | |
| RAMP1 | 10267 | | N/A | |
| RARA | 5914 | | N/A | |
| RASSF7 | 8045 | | N/A | |
| RSAD2 | 91543 | | N/A | |
| RTP4 | 64108 | | N/A | |
| SAMD9 | 54809 | | N/A | |
| SAMD9L | 219285 | | N/A | |
| SASH3 | 54440 | | N/A | |
| SCD | 6319 | | N/A | |
| SELL | 6402 | | N/A | |
| SIX1 | 6495 | AS1_SIX1 | Hs539969.0C4n3_at | 8 |
| SLAMF7 | 57823 | | N/A | |
| SLC12A2 | 6558 | | N/A | |
| SLC9A3R1 | 9368 | AS1_SLC9A3R1 | Hs396783.3C1n4_at | 9 |
| SPOCK2 | 9806 | | N/A | |
| SQLE | 6713 | | N/A | |
| ST20 | 400410 | | N/A | |
| ST6GALNAC2 | 10610 | | N/A | |
| STAT1 | 6772 | AS1_STAT1 | BRMX.13670C1n2_at | 10 |
| STRA13 | 201254 | | N/A | |
| SUSD4 | 55061 | | N/A | |
| SYT12 | 91683 | | N/A | |
| TAP1 | 6890 | | N/A | |
| TBC1D10C | 374403 | | N/A | |
| TNFRSF13B | 23495 | | N/A | |
| TNFSF10 | 8743 | | N/A | |
| TOB1 | 10140 | AS1_TOB1 | BRAD.30243_at | 11 |
| TOM1L1 | 10040 | | N/A | |
| TRIM22 | 10346 | | N/A | |
| UBD | 10537 | AS1_UBD | BRMX.941C2n2_at | 12 |
| UBE2T | 29089 | | N/A | |
| UCK2 | 7371 | | N/A | |
| USP18 | 11274 | | N/A | |
| VNN2 | 8875 | | N/A | |
| XAF1 | 54739 | | N/A | |
| ZWINT | 11130 | | N/A | |
| | | AS1_C1QC | BRMX.4154C1n3_s_at | 13 |
| | | AS1_C2orf14 | BRAD.39498_at | 14 |
| | | AS1_EPSTI1 | BRAD.34868_s_at | 15 |
| | | AS1_GALNT6 | 5505575.0C1n42_at | 16 |
| | | AS1_HIST1H4H | BREM.1442_at | 17 |
| | | AS1_HIST2H4B | BRHP.827_s_at | 18 |
| | | AS2_HIST2H4B | BRRS.18322_s_at | 19 |
| | | AS3_HIST2H4B | BRRS.18792_s_at | 20 |
| | | AS1_KIAA1244 | Hs632609.0C1n37_at | 21 |
| | | AS1_LOC100287927 | Hs449575.0C1n22_at | 22 |
| | | AS1_LOC100291682 | BRAD.18827_s_at | 23 |
| | | AS1_LOC100293679 | BREM.2466_s_at | 24 |

TABLE 1B
Original list of genes tested in breast cancer
and mapped to OAC (via Xcel array)
Novel genes

| Gene symbol | SEQ ID NO: |
|---|---|
| BRAD.2605_at | 25 |
| BRAD.33618_at | 26 |
| BRAD.36579_s_at | 27 |
| BRAD1_5440961_s_at | 28 |
| BRAD1_66786229_s_at | 29 |
| BREM.2104_at | 30 |
| BRAG_AK097020.1_at | 31 |
| BRAD.20415_at | 32 |
| BRAD.29668_at | 33 |
| BRAD.30228_at | 34 |
| BRAD.34830_at | 35 |
| BRAD.37011_s_at | 36 |
| BRAD.37762_at | 37 |
| BRAD.40217_at | 38 |
| BRAD1_4307876_at | 39 |
| BREM.2505_at | 40 |
| Hs149363.0CB4n5_s_at | 41 |
| Hs172587.9C1n9_at | 42 |
| Hs271955.16C1n9_at | 43 |
| Hs368433.18C1n6_at | 44 |
| Hs435736.0C1n27_s_at | 45 |
| Hs493096.15C1n6_at | 46 |
| Hs493096.2C1n15_s_at | 47 |
| Hs592929.0CB2n8_at | 48 |
| Hs79953.0C1n23_at | 49 |
| BRMX.2377C1n3_at | 50 |

All or a portion of the biomarkers recited in Tables 1A and/or 1B and/or 3 may be used in a predictive biomarker panel. For example, biomarker panels selected from the biomarkers in Tables 1A and/or 1B and/or 3 can be generated using the methods provided herein and can comprise between one, and all of the biomarkers set forth in Tables 1A and/or 1B and/or 3 and each and every combination in between (e.g., four selected biomarkers, 16 selected biomarkers, 74 selected biomarkers, etc.). In some embodiments, the predictive biomarker set comprises at least 5, 10, 20, 40, 60, 100, 150, 200, or 300 or more biomarkers. In other embodiments, the predictive biomarker set comprises no more than 5, 10, 20, 40, 60, 100, 150, 200, 300, 400, 500, 600 or 700 biomarkers. In some embodiments, the predictive biomarker set includes a plurality of biomarkers listed in Tables 1A and/or 1B and/or 3. In some embodiments the predictive biomarker set includes at least about 1%, about 5%, about 10%, about 20%, about 30%, about 40%, about 50%, about 60%, about 70%, about 80%, about 90%, about 95%, about 96%, about 97%, about 98%, or about 99% of the biomarkers listed in Tables 1A and/or 1B and/or 3. Selected predictive biomarker sets can be assembled from the predictive biomarkers provided using methods described herein and analogous methods known in the art. In one embodiment, the biomarker panel contains all 50 biomarkers in Table 1 or all biomarkers listed in Table 3. In another embodiment, the biomarker panel corresponds to the 40 or 44 gene panel described in Tables 2A and 2B.

Predictive biomarker sets may be defined in combination with corresponding scalar weights on the real scale with varying magnitude, which are further combined through linear or non-linear, algebraic, trigonometric or correlative means into a single scalar value via an algebraic, statistical learning, Bayesian, regression, or similar algorithms which together with a mathematically derived decision function on the scalar value provide a predictive model by which expression profiles from samples may be resolved into discrete classes of responder or non-responder, resistant or non-resistant, to a specified drug or drug class. Such predictive models, including biomarker membership, are developed by learning weights and the decision threshold, optimized for sensitivity, specificity, negative and positive predictive values, hazard ratio or any combination thereof, under cross-validation, bootstrapping or similar sampling techniques, from a set of representative expression profiles from historical patient samples with known drug response and/or resistance or with known molecular subtype (i.e. DDRD) classification.

In one embodiment, the biomarkers are used to form a weighted sum of their signals, where individual weights can be positive or negative. The resulting sum (“decisive function”) is compared with a pre-determined reference point or value. The comparison with the reference point or value may be used to diagnose, or predict a clinical condition or outcome.

As described above, one of ordinary skill in the art will appreciate that the biomarkers included in the classifier or classifiers provided in Tables 1A and/or 1B and/or 3 will carry unequal weights in a classifier for responsiveness or resistance to a therapeutic agent. Therefore, while as few as one sequence may be used to diagnose or predict an outcome such as responsiveness to therapeutic agent, the specificity and sensitivity or diagnosis or prediction accuracy may increase using more sequences.

As used herein, the term “weight” refers to the relative importance of an item in a statistical calculation. The weight of each biomarker in a gene expression classifier may be determined on a data set of patient samples using analytical methods known in the art. Gene specific bias values may also be applied. Gene specific bias may be required to mean centre each gene in the classifier relative to a training data set, as would be understood by one skilled in the art. Specific bias values are presented in Table 4 and may be applied according to the methods and kits of the invention.

In one embodiment the biomarker panel is directed to the 40 biomarkers detailed in Table 2A with corresponding ranks and weights detailed in the table or alternative rankings and weightings, depending, for example, on the disease setting. In another embodiment, the biomarker panel is directed to the 44 biomarkers detailed in Table 2B with corresponding ranks and weights detailed in the table or alternative rankings and weightings, depending, for example, on the disease setting. Tables 2A and 2B rank the biomarkers in order of decreasing weight in the classifier, defined as the rank of the average weight in the compound decision score function measured under cross-validation. In another embodiment, the biomarker panel is directed to the 44 biomarkers detailed in Table 4 with corresponding ranks, weights and bias detailed in the table or alternative rankings, weightings and bias, depending, for example, on the disease setting.

TABLE 2A
Gene IDs and EntrezGene IDs for 40-gene DDRD classifier model
with associated ranking and weightings
DDRD classifier 40 gene model

| Rank | Genes Symbol | EntrezGene ID | Weights |
|---|---|---|---|
| 1 | GBP5 | 115362 | 0.022389581 |
| 2 | CXCL10 | 3627 | 0.021941734 |
| 3 | IDO1 | 3620 | 0.020991115 |
| 4 | MX1 | 4599 | 0.020098675 |
| 5 | IFI44L | 10964 | 0.018204957 |
| 6 | CD2 | 914 | 0.018080661 |
| 7 | PRAME | 23532 | 0.016850837 |
| 8 | ITGAL | 3683 | 0.016783359 |
| 9 | LRP4 | 4038 | −0.015129969 |
| 10 | SP140L | 93349 | 0.014646025 |
| 11 | APOL3 | 80833 | 0.014407174 |
| 12 | FOSB | 2354 | −0.014310521 |
| 13 | CDR1 | 1038 | −0.014209848 |
| 14 | RSAD2 | 91543 | 0.014177132 |
| 15 | TSPAN7 | 7102 | −0.014111562 |
| 16 | RAC2 | 5880 | 0.014093627 |
| 17 | FYB | 2533 | 0.01400475 |
| 18 | KLHDC7B | 113730 | 0.013298413 |
| 19 | GRB14 | 2888 | 0.013031204 |
| 20 | KIF26A | 26153 | −0.012942351 |
| 21 | CD274 | 29126 | 0.012651964 |
| 22 | CD109 | 135228 | −0.012239425 |
| 23 | ETV7 | 51513 | 0.011787297 |
| 24 | MFAP5 | 8076 | −0.011480443 |
| 25 | OLFM4 | 10562 | −0.011130113 |
| 26 | PI15 | 51050 | −0.010904326 |
| 27 | FAM19A5 | 25817 | −0.010500936 |
| 28 | NLRC5 | 84166 | 0.009593449 |
| 29 | EGR1 | 1958 | −0.008947963 |
| 30 | ANXA1 | 301 | −0.008373991 |
| 31 | CLDN10 | 9071 | −0.008165127 |
| 32 | ADAMTS4 | 9507 | −0.008109892 |
| 33 | ESR1 | 2099 | 0.007524594 |
| 34 | PTPRC | 5788 | 0.007258669 |
| 35 | EGFR | 1956 | −0.007176203 |
| 36 | NAT1 | 9 | 0.006165534 |
| 37 | LATS2 | 26524 | −0.005951091 |
| 38 | CYP2B6 | 1555 | 0.005838391 |
| 39 | PPP1R1A | 5502 | −0.003898835 |
| 40 | TERF1P1 | 348567 | 0.002706847 |

TABLE 2B
Gene IDs and EntrezGene IDs for 44-gene DDRD classifier
model with associated ranking and weightings
DDRD Classifier-44 Gene Model (NA: genomic sequence)

| Rank | Gene symbol | EntrezGene ID | Weight |
|---|---|---|---|
| 1 | CXCL10 | 3627 | 0.023 |
| 2 | MX1 | 4599 | 0.0226 |
| 3 | IDO1 | 3620 | 0.0221 |
| 4 | IFI44L | 10964 | 0.0191 |
| 5 | CD2 | 914 | 0.019 |
| 6 | GBP5 | 115362 | 0.0181 |
| 7 | PRAME | 23532 | 0.0177 |
| 8 | ITGAL | 3683 | 0.0176 |
| 9 | LRP4 | 4038 | −0.0159 |
| 10 | APOL3 | 80833 | 0.0151 |
| 11 | CDR1 | 1038 | −0.0149 |
| 12 | FYB | 2533 | −0.0149 |
| 13 | TSPAN7 | 7102 | 0.0148 |
| 14 | RAC2 | 5880 | −0.0148 |
| 15 | KLHDC7B | 113730 | 0.014 |
| 16 | GRB14 | 2888 | 0.0137 |
| 17 | AC138128.1 | N/A | −0.0136 |
| 18 | KIF26A | 26153 | −0.0136 |
| 19 | CD274 | 29126 | 0.0133 |
| 20 | CD109 | 135228 | −0.0129 |
| 21 | ETV7 | 51513 | 0.0124 |
| 22 | MFAP5 | 8076 | −0.0121 |
| 23 | OLFM4 | 10562 | −0.0117 |
| 24 | PI15 | 51050 | −0.0115 |
| 25 | FOSB | 2354 | −0.0111 |
| 26 | FAM19A5 | 25817 | 0.0101 |
| 27 | NLRC5 | 84166 | −0.011 |
| 28 | PRICKLE1 | 144165 | −0.0089 |
| 29 | EGR1 | 1958 | −0.0086 |
| 30 | CLDN10 | 9071 | −0.0086 |
| 31 | ADAMTS4 | 9507 | −0.0085 |
| 32 | SP140L | 93349 | 0.0084 |
| 33 | ANXA1 | 301 | −0.0082 |
| 34 | RSAD2 | 91543 | 0.0081 |
| 35 | ESR1 | 2099 | 0.0079 |
| 36 | IKZF3 | 22806 | 0.0073 |
| 37 | OR2I1P | 442197 | 0.007 |
| 38 | EGFR | 1956 | −0.0066 |
| 39 | NAT1 | 9 | 0.0065 |
| 40 | LATS2 | 26524 | −0.0063 |
| 41 | CYP2B6 | 1555 | 0.0061 |
| 42 | PTPRC | 5788 | 0.0051 |
| 43 | PPP1R1A | 5502 | −0.0041 |
| 44 | AL137218.1 | N/A | −0.0017 |

Table 3 presents the probe sets from the Xcel Array (Almac) that represent the genes in Table 2A and 2B with reference to their sequence ID numbers.

TABLE 3
Probe set IDs and SEQ Numbers for target sequences of genes
contained in 44-gene signature as mapped to Xcel platform

| | | SEQ ID NO: |
|---|---|---|
| Gene | Probeset ID | of Target sequence |
| AC138128.1 | NONMATCH | #N/A |
| ADAMTS4 | ADXEC.29185.C1_at | 51 |
| ADAMTS4 | ADXECAD.1557_at | 52 |
| ADAMTS4 | ADXECAD.1557_x_at | 53 |
| ADAMTS4 | ADXECNTDJ.9649_at | 54 |
| AL137218.1 | ADXECADA.15298_x_at | 55 |
| ANXA1 | ADXEC.961.C1_at | 56 |
| ANXA1 | ADXEC.961.C2_s_at | 57 |
| ANXA1 | ADXEC.961.C3_at | 58 |
| ANXA1 | ADXECAD.8396_at | 59 |
| APOL3 | ADXEC.11171.C1_s_at | 60 |
| CD109 | ADXEC.11145.C1_s_at | 61 |
| CD109 | ADXEC.11777.C1_at | 62 |
| CD109 | ADXEC.12292.C1_at | 63 |
| CD2 | ADXEC.7301.C1-a_s_at | 64 |
| CD2 | ADXEC.7301.C1_at | 65 |
| CD2 | ADXECEMUTR.6872_at | 66 |
| CD2 | ADXECRS.12205_s_at | 67 |
| CD274 | ADXEC.11136.C1_at | 68 |
| CD274 | ADXEC.23232.C1_at | 69 |
| CD274 | ADXECNTDJ.4196_s_at | 70 |
| CD274 | ADXECNTDJ.4198_s_at | 71 |
| CDR1 | ADXECRS.7695_s_at | 72 |
| CLDN10 | ADXEC.19503.C1_s_at | 73 |
| CLDN10 | ADXECEMUTR.6957_at | 74 |
| CLDN10 | ADXECRS.17517_s_at | 75 |
| CXCL10 | ADXEC.11676.C1_at | 76 |
| CYP2B6 | ADXEC.20112.C1_s_at | 77 |
| CYP2B6 | ADXECAD.18663_x_at | 78 |
| CYP2B6 | ADXLCEC.9263.C1_at | 79 |
| EGFR | ADXEC.14093.C1_at | 80 |
| EGFR | ADXEC.1866.C1_at | 81 |
| EGFR | ADXEC.1866.C1_x_at | 82 |
| EGFR | ADXEC.21483.C1_at | 83 |
| EGFR | ADXEC.23775.C1_at | 84 |
| EGFR | ADXEC.31869.C1_at | 85 |
| EGFR | ADXEC.4451.C1_at | 86 |
| EGFR | ADXECAD.18126_at | 87 |
| EGFR | ADXECAD.19259_at | 88 |
| EGFR | ADXECADA.15206_at | 89 |
| EGFR | ADXECADA.21225_s_at | 90 |
| EGFR | ADXECADA.8307_at | 91 |
| EGFR | ADXECEMUTR.2965_at | 92 |
| EGFR | ADXECEMUTR.3575_at | 93 |
| EGFR | ADXECNTDJ.6255_at | 94 |
| EGFR | ADXECNTDJ.6256_at | 95 |
| EGFR | ADXECNTDJ.6256_x_at | 96 |
| EGFR | ADXECRS.19907_at | 97 |
| EGFR | ADXECRS.19907_s_at | 98 |
| EGFR | ADXECRS.24032_at | 99 |
| EGFR | ADXLCEC.7900.C1_at | 100 |
| EGFR | ADXPCEC.14538.C1_at | 101 |
| EGR1 | ADXEC.2432.C2_s_at | 102 |
| EGR1 | ADXEC.2432.C4_at | 103 |
| EGR1 | ADXEC.2432.C6-a_s_at | 104 |
| ESR1 | ADXEC.27541.C1_at | 105 |
| ESR1 | ADXEC.29140.C1_s_at | 106 |
| ESR1 | ADXEC.33997.C1_at | 107 |
| ESR1 | ADXECAD.12370_s_at | 108 |
| ESR1 | ADXECAD.18631_at | 109 |
| ESR1 | ADXECAD.24092_s_at | 110 |
| ESR1 | ADXECADA.11317_s_at | 111 |
| ESR1 | ADXECADA.9299_at | 112 |
| ESR1 | ADXECNTDJ.3778_at | 113 |
| ESR1 | ADXECNTDJ.3779_at | 114 |
| ESR1 | ADXOCEC.10271.C1_at | 115 |
| ESR1 | ADXOCEC.10271.C1_x_at | 116 |
| ESR1 | ADXOCEC.9813.C1_at | 117 |
| ETV7 | ADXEC.745.C1_s_at | 118 |
| ETV7 | ADXECEMUTR.534_s_at | 119 |
| FAM19A5 | ADXEC.10689.C1_at | 120 |
| FAM19A5 | ADXEC.13789.C1_at | 121 |
| FAM19A5 | ADXEC.13789.C1_s_at | 122 |
| FAM19A5 | ADXEC.13789.C1_x_at | 123 |
| FAM19A5 | ADXECADA.11183_at | 124 |
| FAM19A5 | ADXECADA.11183_s_at | 125 |
| FAM19A5 | ADXECADA.11183_x_at | 126 |
| FAM19A5 | ADXECNTDJ.10271_at | 127 |
| FOSB | ADXEC.34273.C1_at | 128 |
| FOSB | ADXEC.34273.C1_x_at | 129 |
| FOSB | ADXEC.9157.C1-a_s_at | 130 |
| FOSB | ADXEC.9157.C1_at | 131 |
| FOSB | ADXECNTDJ.4222_s_at | 132 |
| FOSB | ADXECNTDJ.4223_at | 133 |
| FOSB | ADXECNTDJ.4223_x_at | 134 |
| FOSB | ADXPCEC.11652.C1_x_at | 135 |
| FYB | ADXECAD.24300_s_at | 136 |
| FYB | ADXECADA.2898_at | 137 |
| FYB | ADXECNTDJ.82_s_at | 138 |
| GBP5 | ADXEC.6891.C2_at | 139 |
| GBP5 | ADXEC.6891.C2_s_at | 140 |
| GBP5 | ADXEC.8878.C1_at | 141 |
| GRB14 | ADXEC.13641.C1_s_at | 142 |
| IDO1 | ADXEC.20415.C1-a_s_at | 143 |
| IFI44L | ADXEC.30980.C1_at | 144 |
| IFI44L | ADXEC.30980.C1_x_at | 145 |
| IFI44L | ADXEC.6079.C1_at | 146 |
| IFI44L | ADXEC.6079.Cl_x_at | 147 |
| IFI44L | ADXOCEC.12110.C2_s_at | 148 |
| IFI44L | ADXOCEC.9547.C1_at | 149 |
| IFI44L | ADXOCEC.9547.C1_x_at | 150 |
| IKZF3 | ADXEC.22688.C1_at | 151 |
| IKZF3 | ADXEC.32096.C1_at | 152 |
| IKZF3 | ADXEC.32096.C1_x_at | 153 |
| IKZF3 | ADXECAD.25262_s_at | 154 |
| IKZF3 | ADXECADA.10727_at | 155 |
| IKZF3 | ADXECRS.658_s_at | 156 |
| ITGAL | ADXEC.7237.C1_s_at | 157 |
| ITGAL | ADXECADA.387_x_at | 158 |
| KIF26A | ADXEC.10112.C1_at | 159 |
| KIF26A | ADXEC.19112.C1_s_at | 160 |
| KLHDC7B | ADXEC.11833.C1_at | 161 |
| KLHDC7B | ADXECADA.94_at | 162 |
| LATS2 | ADXEC.11588.C1_s_at | 163 |
| LATS2 | ADXEC.8316.C2_s_at | 164 |
| LATS2 | ADXECAD.19393_at | 165 |
| LRP4 | ADXEC.13953.C1_at | 166 |
| LRP4 | ADXEC.15783.C1_at | 167 |
| LRP4 | ADXECADA.18233_at | 168 |
| MFAP5 | ADXEC.18200.C1_at | 169 |
| MFAP5 | ADXEC.8579.C1-a_s_at | 170 |
| MFAP5 | ADXEC.8579.C1_at | 171 |
| MFAP5 | ADXEC.8579.C2_s_at | 172 |
| MX1 | ADXEC.6683.C1_at | 173 |
| MX1 | ADXEC.6683.C1_s_at | 174 |
| MX1 | ADXEC.6842.C2_at | 175 |
| MX1 | ADXEC.6842.C2_x_at | 176 |
| NAT1 | ADXEC.20034.C1-a_s_at | 177 |
| NAT1 | ADXEC.20034.C1_at | 178 |
| NAT1 | ADXEC.20034.C2_s_at | 179 |
| NAT1 | ADXECEMUTR.4521_s_at | 180 |
| NAT1 | ADXECNTDJ.5862_s_at | 181 |
| NAT1 | ADXECNTDJ.5864_s_at | 182 |
| NAT1 | ADXECNTDJ.5866_s_at | 183 |
| NAT1 | ADXECNTDJ.5867_at | 184 |
| NAT1 | ADXECNTDJ.5868_at | 185 |
| NLRC5 | ADXEC.23051.C1_s_at | 186 |
| NLRC5 | ADXEC.5068.C1_at | 187 |
| NLRC5 | ADXECEMUTR.5074_at | 188 |
| NLRC5 | ADXECEMUTR.5074_s_at | 189 |
| NLRC5 | ADXECNTDJ.5048_s_at | 190 |
| OLFM4 | ADXEC.8457.C1-a_s_at | 191 |
| OLFM4 | ADXEC.8457.C1_s_at | 192 |
| OR2I1P | ADXECAD.16836_at | 193 |
| OR2I1P | ADXECAD.16836_s_at | 194 |
| PI15 | ADXEC.29833.C1-a_s_at | 195 |
| PI15 | ADXEC.29833.C1_at | 196 |
| PI15 | ADXEC.29833.C1_s_at | 197 |
| PI15 | ADXEC.7703.C1_at | 198 |
| PI15 | ADXEC.7703.C1_x_at | 199 |
| PI15 | ADXECAD.23062_at | 200 |
| PPP1R1A | ADXEC.14340.C1_at | 201 |
| PPP1R1A | ADXEC.15744.C1_at | 202 |
| PRAME | ADXEC.11333.C1_at | 203 |
| PRAME | ADXEC.11333.C1_x_at | 204 |
| PRICKLE1 | ADXEC.9436.C1_at | 205 |
| PRICKLE1 | ADXEC.9436.C1_x_at | 206 |
| PRICKLE1 | ADXECAD.6243_s_at | 207 |
| PRICKLE1 | ADXECAD.8320_at | 208 |
| PRICKLE1 | ADXECRS.11172_s_at | 209 |
| PRICKLE1 | ADXECRS.18104_s_at | 210 |
| PTPRC | ADXEC.8915.C1-a_s_at | 211 |
| PTPRC | ADXEC.8915.C1_at | 212 |
| PTPRC | ADXECAD.17697_at | 213 |
| PTPRC | ADXECADA.4026_at | 214 |
| PTPRC | ADXECADA.52_at | 215 |
| PTPRC | ADXECNTDJ.2722_s_at | 216 |
| PTPRC | ADXECNTDJ.2723_s_at | 217 |
| RAC2 | ADXEC.15369.C1_s_at | 218 |
| RSAD2 | ADXEC.8308.C1-a_s_at | 219 |
| RSAD2 | ADXEC.8308.C1_at | 220 |
| RSAD2 | ADXECAD.11200_at | 221 |
| RSAD2 | ADXECADA.13258_s_at | 222 |
| RSAD2 | ADXECNTDJ.5191_at | 223 |
| RSAD2 | ADXECRS.4576_s_at | 224 |
| SP140L | ADXEC.31390.C1_at | 225 |
| SP140L | ADXECADA.3222_at | 226 |
| TSPAN7 | ADXEC.12786.C1_at | 227 |
| TSPAN7 | ADXECADA.9258_at | 228 |
| TSPAN7 | ADXECADA.9258_x_at | 229 |
| TSPAN7 | ADXECNTDJ.7964_at | 230 |

TABLE 4
Gene Symbols for 44-gene DDRD classifier model
with associated weightings and bias

| Gene symbol | Weight | Bias |
|---|---|---|
| AC138128.1 | −0.0136 | 1.4071 |
| ADAMTS4 | −0.0085 | 1.9569 |
| AL137218.1 | −0.0017 | −1.1744 |
| ANXA1 | −0.0082 | 2.0015 |
| APOL3 | 0.0151 | 2.2036 |
| CD109 | −0.0129 | 0.9477 |
| CD2 | 0.0190 | 4.0904 |
| CD274 | 0.0133 | 1.3730 |
| CDR1 | −0.0149 | 4.7979 |
| CLDN10 | −0.0086 | −0.3446 |
| CXCL10 | 0.0230 | 2.0393 |
| CYP2B6 | 0.0061 | 0.9218 |
| EGFR | −0.0066 | −0.1767 |
| EGR1 | −0.0086 | 2.1865 |
| ESR1 | 0.0079 | 0.8512 |
| ETV7 | 0.0124 | 1.4678 |
| FAM19A5 | −0.0110 | 0.4137 |
| FOSB | −0.0111 | 1.8589 |
| FYB | 0.0149 | 1.5618 |
| GBP5 | 0.0181 | 1.3977 |
| GRB14 | 0.0137 | 0.2696 |
| IDO1 | 0.0221 | 0.7257 |
| IFI44L | 0.0191 | 1.1758 |
| IKZF3 | 0.0073 | −0.5899 |
| ITGAL | 0.0176 | 3.2161 |
| KIF26A | −0.0136 | 2.0504 |
| KLHDC7B | 0.0140 | 1.4395 |
| LATS2 | −0.0063 | 0.4863 |
| LRP4 | −0.0159 | 0.3065 |
| MFAP5 | −0.0121 | 2.6992 |
| MX1 | 0.0226 | 3.4355 |
| NAT1 | 0.0065 | −0.7973 |
| NLRC5 | 0.0101 | 2.2686 |
| OLFM4 | −0.0117 | 0.6367 |
| OR2I1P | 0.0070 | −1.3024 |
| PI15 | −0.0115 | 0.3355 |
| PPP1R1A | −0.0041 | 1.7637 |
| PRAME | 0.0177 | 2.2499 |
| PRICKLE1 | −0.0089 | 1.7702 |
| PTPRC | 0.0051 | −1.1182 |
| RAC2 | 0.0148 | 3.0364 |
| RSAD2 | 0.0081 | 1.4489 |
| SP140L | 0.0084 | 0.5505 |
| TSPAN7 | −0.0148 | 1.6584 |

In different embodiments, subsets of the biomarkers listed in Tables 1A and/or 1B, Table 2A and/or Table 2B and/or Tables 3 and/or 4 may be used in the methods described herein. These subsets include but are not limited to biomarkers ranked 1-2, 1-3, 1-4, 1-5, 1-10, 1-20, 1-30, 1-40, 1-44, 6-10, 11-15, 16-20, 21-25, 26-30, 31-35, 36-40, 36-44, 11-20, 21-30, 31-40, and 31-44 in Table 2A or Table 2B.

In one aspect, therapeutic responsiveness is predicted in an individual by conducting an assay on a test (biological) sample from the individual and detecting biomarker values that each correspond to at least one of the biomarkers GBP5, CXCL10, IDO1 and MX1 and at least N additional biomarkers selected from the list of biomarkers in Table 2B, wherein N equals 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36. As used herein, the term “biomarker” can refer to a gene, an mRNA, cDNA, an antisense transcript, a miRNA, a polypeptide, a protein, a protein fragment, or any other nucleic acid sequence or polypeptide sequence that indicates either gene expression levels or protein production levels. In some embodiments, when referring to a biomarker of CXCL10, IDO1, CD2, GBP5, PRAME, ITGAL, LRP4, APOL3, CDR1, FYB, TSPAN7, RAC2, KLHDC7B, GRB14, AC138128.1, KIF26A, CD274, ETV7, MFAP5, OLFM4, PI15, FOSB, FAM19A5, NLRC5, PRICKLE1, EGR1, CLDN10, ADAMTS4, SP140L, ANXA1, RSAD2, ESR1, IKZF3, OR2I1P, EGFR, NAT1, LATS2, CYP2B6, PTPRC, PPP1R1A, or AL137218.1, the biomarker comprises an mRNA of CXCL10, IDO1, CD2, GBP5, PRAME, ITGAL, LRP4, APOL3, CDR1, FYB, TSPAN7, RAC2, KLHDC7B, GRB14, AC138128.1, KIF26A, CD274, ETV7, MFAP5, OLFM4, PI15, FOSB, FAM19A5, NLRC5, PRICKLE1, EGR1, CLDN10, ADAMTS4, SP140L, ANXA1, RSAD2, ESR1, IKZF3, OR2I1P, EGFR, NAT1, LATS2, CYP2B6, PTPRC, PPP1R1A, or AL137218.1, respectively. In further or other embodiments, when referring to a biomarker of MX1, GBP5, IFI44L, BIRC3, IGJ, IQGAP3, LOC100294459, SIX1, SLC9A3R1, STAT1, TOB1, UBD, C1QC, C2orf14, EPSTI, GALNT6, HIST1H4H, HIST2H4B, KIAA1244, LOC100287927, LOC100291682, or LOC100293679, the biomarker comprises an antisense transcript of MX1, IFI44L, GBP5, BIRC3, IGJ, IQGAP3, LOC100294459, SIX1, SLC9A3R1, STAT1, TOB1, UBD, C1QC, C2orf14, EPSTI, GALNT6, HIST1H4H, HIST2H4B, KIAA1244, LOC100287927, LOC100291682, or LOC100293679, respectively.

In a further aspect, therapeutic responsiveness is predicted, or a cancer diagnosis is indicated, in an individual by conducting an assay on a test (biological) sample from the individual and detecting biomarker values that each correspond to the biomarkers GBP5, CXCL10, IDO1 and MX1 and one of at least N additional biomarkers selected from the list of biomarkers in Table 2B, wherein N equals 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36. In a further aspect, therapeutic responsiveness is predicted, or a cancer diagnosis is indicated, in an individual by conducting an assay on a test (biological) sample from the individual and detecting biomarker values that each correspond to the biomarker GBP5 and one of at least N additional biomarkers selected from the list of biomarkers in Table 2B, wherein N equals 2, 3, 4, 5, 6, 7, 8, 9, 10, 11, 12, 13, 14, 15, 16, 17, 18, 29, 20, 21, 22, 23, 24, 25, 26, 27, 28, 29, 30, 31, 32, 33, 34, 35, 36, 37, 38 or 39. In a further aspect, therapeutic responsiveness is predicted, or a cancer diagnosis is indicated, in an individual by conducting an assay on a test (biological) sample from the individual and detecting biomarker values that each correspond to the biomarker CXCL10 and one of at least N additional biomarkers selected from the list of biomarkers in Table 2B, wherein N equals 2, 3, 4, 5, 6, 7, 8, 9, 10, 11, 12, 13, 14, 15, 16, 17, 18, 29, 20, 21, 22, 23, 24, 25, 26, 27, 28, 29, 30, 31, 32, 33, 34, 35, 36, 37, 38 or 39. In a further aspect, therapeutic responsiveness is predicted, or a cancer diagnosis is indicated, in an individual by conducting an assay on a test (biological) sample from the individual and detecting biomarker values that each correspond to the biomarker IDO1 and one of at least N additional biomarkers selected from the list of biomarkers in Table 2B, wherein N equals 2, 3, 4, 5, 6, 7, 8, 9, 10, 11, 12, 13, 14, 15, 16, 17, 18, 29, 20, 21, 22, 23, 24, 25, 26, 27, 28, 29, 30, 31, 32, 33, 34, 35, 36, 37, 38 or 39. In a further aspect, therapeutic responsiveness is predicted, or a cancer diagnosis is indicated, in an individual by conducting an assay on a test (biological) sample from the individual and detecting biomarker values that each correspond to the biomarker MX-1 and one of at least N additional biomarkers selected from the list of biomarkers in Table 2B, wherein N equals 2, 3, 4, 5, 6, 7, 8, 9, 10, 11, 12, 13, 14, 15, 16, 17, 18, 29, 20, 21, 22, 23, 24, 25, 26, 27, 28, 29, 30, 31, 32, 33, 34, 35, 36, 37, 38 or 39.

In a further aspect, therapeutic responsiveness is predicted, or a cancer diagnosis is indicated, in an individual by conducting an assay on a test (biological) sample from the individual and detecting biomarker values that each correspond to at least two of the biomarkers CXCL10, MX1, IDO1 and IFI44L and at least N additional biomarkers selected from the list of biomarkers in Table 2B, wherein N equals 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40. In a further aspect, therapeutic responsiveness is predicted, or a cancer diagnosis is indicated, in an individual by conducting an assay on a test (biological) sample from the individual and detecting biomarker values that each correspond to the biomarkers CXCL10, MX1, IDO1 and IFI44L and one of at least N additional biomarkers selected from the list of biomarkers in Table 2B, wherein N equals 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40. In a further aspect, therapeutic responsiveness is predicted, or a cancer diagnosis is indicated, in an individual by conducting an assay on a test (biological) sample from the individual and detecting biomarker values that each correspond to the biomarker CXCL10 and one of at least N additional biomarkers selected from the list of biomarkers in Table 2B, wherein N equals 2, 3, 4, 5, 6, 7, 8, 9, 10, 11, 12, 13, 14, 15, 16, 17, 18, 29, 20, 21, 22, 23, 24, 25, 26, 27, 28, 29, 30, 31, 32, 33, 34, 35, 36, 37, 38, 39, 40, 41, 42 or 43. In a further aspect, therapeutic responsiveness is predicted, or a cancer diagnosis is indicated, in an individual by conducting an assay on a test (biological) sample from the individual and detecting biomarker values that each correspond to the biomarker MX1 and one of at least N additional biomarkers selected from the list of biomarkers in Table 2B, wherein N equals 2, 3, 4, 5, 6, 7, 8, 9, 10, 11, 12, 13, 14, 15, 16, 17, 18, 29, 20, 21, 22, 23, 24, 25, 26, 27, 28, 29, 30, 31, 32, 33, 34, 35, 36, 37, 38, 39, 40, 41, 42 or 43. In a further aspect, therapeutic responsiveness is predicted, or a cancer diagnosis is indicated, in an individual by conducting an assay on a test (biological) sample from the individual and detecting biomarker values that each correspond to the biomarker IDO1 and one of at least N additional biomarkers selected from the list of biomarkers in Table 2B, wherein N equals 2, 3, 4, 5, 6, 7, 8, 9, 10, 11, 12, 13, 14, 15, 16, 17, 18, 29, 20, 21, 22, 23, 24, 25, 26, 27, 28, 29, 30, 31, 32, 33, 34, 35, 36, 37, 38, 39, 40, 41, 42 or 43. In a further aspect, therapeutic responsiveness is predicted, or a cancer diagnosis is indicated, in an individual by conducting an assay on a test (biological) sample from the individual and detecting biomarker values that each correspond to the biomarker IFI44L and one of at least N additional biomarkers selected from the list of biomarkers in Table 2B, wherein N equals 2, 3, 4, 5, 6, 7, 8, 9, 10, 11, 12, 13, 14, 15, 16, 17, 18, 29, 20, 21, 22, 23, 24, 25, 26, 27, 28, 29, 30, 31, 32, 33, 34, 35, 36, 37, 38, 39, 40, 41, 42 or 43.

In other embodiments, the target sequences listed in Tables 1A, 1B and/or 3, or subsets thereof, may be used in the methods described herein. The target sequences may be utilised for the purposes of designing primers and/or probes which hybridize to the target sequences. Design of suitable primers and/or probes is within the capability of one skilled in the art once the target sequence is identified. Various primer design tools are freely available to assist in this process, such as the NCBI Primer-BLAST tool; see Ye et al, BMC Bioinformatics. 13:134 (2012). The primers and/or probes may be designed such that they hybridize to the target sequence under stringent conditions (as defined herein). Primers and/or probes may be at least 15, 16, 17, 18, 19, 20, 21, 22, 23, 24 or 25 (or more) nucleotides in length. It should be understood that each subset can include multiple primers and/or probes directed to the same biomarker. The tables show in some cases multiple target sequences within the same overall gene. Such primers and/or probes may be included in kits useful for performing the methods of the invention. The kits may be array or PCR based kits for example and may include additional reagents, such as a polymerase and/or dNTPs for example.

### Measuring Gene Expression Using Classifier Models

A variety of methods have been utilized in an attempt to identify biomarkers and diagnose disease. For protein-based markers, these include two-dimensional electrophoresis, mass spectrometry, and immunoassay methods. For nucleic acid markers, these include mRNA expression profiles, microRNA profiles, sequencing, FISH, serial analysis of gene expression (SAGE), methylation profiles, and large-scale gene expression arrays.

When a biomarker indicates or is a sign of an abnormal process, disease or other condition in an individual, that biomarker is generally described as being either over-expressed or under-expressed as compared to an expression level or value of the biomarker that indicates or is a sign of a normal process, an absence of a disease or other condition in an individual. “Up-regulation”, “up-regulated”, “over-expression”, “over-expressed”, and any variations thereof are used interchangeably to refer to a value or level of a biomarker in a biological sample that is greater than a value or level (or range of values or levels) of the biomarker that is typically detected in similar biological samples from healthy or normal individuals. The terms may also refer to a value or level of a biomarker in a biological sample that is greater than a value or level (or range of values or levels) of the biomarker that may be detected at a different stage of a particular disease.

“Down-regulation”, “down-regulated”, “under-expression”, “under-expressed”, and any variations thereof are used interchangeably to refer to a value or level of a biomarker in a biological sample that is less than a value or level (or range of values or levels) of the biomarker that is typically detected in similar biological samples from healthy or normal individuals. The terms may also refer to a value or level of a biomarker in a biological sample that is less than a value or level (or range of values or levels) of the biomarker that may be detected at a different stage of a particular disease.

Further, a biomarker that is either over-expressed or under-expressed can also be referred to as being “differentially expressed” or as having a “differential level” or “differential value” as compared to a “normal” expression level or value of the biomarker that indicates or is a sign of a normal process or an absence of a disease or other condition in an individual. Thus, “differential expression” of a biomarker can also be referred to as a variation from a “normal” expression level of the biomarker.

The terms “differential biomarker expression” and “differential expression” are used interchangeably to refer to a biomarker whose expression is activated to a higher or lower level in a subject suffering from a specific disease, relative to its expression in a normal subject, or relative to its expression in a patient that responds differently to a particular therapy or has a different prognosis. The terms also include biomarkers whose expression is activated to a higher or lower level at different stages of the same disease. It is also understood that a differentially expressed biomarker may be either activated or inhibited at the nucleic acid level or protein level, or may be subject to alternative splicing to result in a different polypeptide product. Such differences may be evidenced by a variety of changes including mRNA levels, miRNA levels, antisense transcript levels, or protein surface expression, secretion or other partitioning of a polypeptide. Differential biomarker expression may include a comparison of expression between two or more genes or their gene products; or a comparison of the ratios of the expression between two or more genes or their gene products; or even a comparison of two differently processed products of the same gene, which differ between normal subjects and subjects suffering from a disease; or between various stages of the same disease. Differential expression includes both quantitative, as well as qualitative, differences in the temporal or cellular expression pattern in a biomarker among, for example, normal and diseased cells, or among cells which have undergone different disease events or disease stages.

In certain embodiments, the expression profile obtained is a genomic or nucleic acid expression profile, where the amount or level of one or more nucleic acids in the sample is determined. In these embodiments, the sample that is assayed to generate the expression profile (i.e. to measure the expression levels of the one or more biomarkers in the sample) employed in the diagnostic or prognostic methods comprises a nucleic acid sample. The nucleic acid sample includes a population of nucleic acids that includes the expression information of the phenotype determinative biomarkers of the cell or tissue being analyzed. In some embodiments, the nucleic acid may include RNA or DNA nucleic acids, e.g., mRNA, cRNA, cDNA etc., so long as the sample retains the expression information of the host cell or tissue from which it is obtained. The sample may be prepared in a number of different ways, as is known in the art, e.g., by mRNA isolation from a cell, where the isolated mRNA is used as isolated, amplified, or employed to prepare cDNA, cRNA, etc., as is known in the field of differential gene expression. Accordingly, determining the level of mRNA in a sample includes preparing cDNA or cRNA from the mRNA and subsequently measuring the cDNA or cRNA. The sample is typically prepared from a cell or tissue harvested from a subject in need of treatment, e.g., via biopsy of tissue, using standard protocols, where cell types or tissues from which such nucleic acids may be generated include any tissue in which the expression pattern of the to be determined phenotype exists, including, but not limited to, disease cells or tissue, body fluids, etc.

The expression profile, representing the measured expression levels of one or more biomarkers in the test sample may be generated from the initial nucleic acid sample using any convenient protocol. While a variety of different manners of generating expression profiles are known, such as those employed in the field of differential gene expression/biomarker analysis, one representative and convenient type of protocol for generating expression profiles is array-based gene expression profile generation protocols. Such applications are hybridization assays in which a surface such as a (glass) chip, on which several probes for each of several thousand genes are immobilised is employed. On these surfaces there are generally multiple target regions within each gene to be analysed, and multiple (usually from 11 to 100) probes per target region. In this way, expression of each gene is evaluated by hybridization to multiple (tens) of probes on the surface. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes one or several probes for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative. The methods may include normalizing the hybridization pattern against a subset of or all other probes on the array.

### Creating a Biomarker Expression Classifier

In one embodiment, the relative expression levels of biomarkers in a cancer tissue are measured to form a gene expression profile. The gene expression profile of a set of biomarkers from a patient tissue sample is summarized in the form of a compound decision score (or test score) and compared to a score threshold that may be mathematically derived from a training set of patient data. The score threshold separates a patient group based on different characteristics such as, but not limited to, responsiveness/non-responsiveness to treatment. The patient training set data is preferably derived from OAC tissue samples having been characterized by prognosis, likelihood of recurrence, long term survival, clinical outcome, treatment response, diagnosis, cancer classification, or personalized genomics profile. Alternatively it may represent a data set from a cohort of patients in which the molecular subtype (DDRD) is well defined and characterised. Expression profiles, and corresponding decision scores from patient samples (test scores) may be correlated with the characteristics of patient samples in the training set that are on the same side of the mathematically derived score decision threshold. The threshold of the linear classifier scalar output may be optimized to maximize the sum of sensitivity and specificity under cross-validation as observed within the training dataset. Alternatively the sensitivity and positive predictive value of the assay may be increased at the expense of the specificity and negative predictive value or vice versa depending on the proposed clinical utility of the test in different disease indications.

The overall expression data for a given sample is normalized using methods known to those skilled in the art in order to correct for differing amounts of starting material, varying efficiencies of the extraction and amplification reactions, etc. Using a linear classifier on the normalized data to make a diagnostic or prognostic call (e.g. responsiveness or resistance to therapeutic agent) effectively means to split the data space, i.e. all possible combinations of expression values for all genes in the classifier, into two disjoint halves by means of a separating hyperplane. This split may be empirically derived on a large set of training examples, for example from patients showing responsiveness or resistance to a therapeutic agent. Without loss of generality, one can assume a certain fixed set of values for all but one biomarker, which would automatically define a threshold value for this remaining biomarker where the decision would change from, for example, responsiveness or resistance to a therapeutic agent. Expression values above this dynamic threshold would then either indicate resistance (for a biomarker with a negative weight) or responsiveness (for a biomarker with a positive weight) to a therapeutic agent. The precise value of this threshold depends on the actual measured expression profile of all other biomarkers within the classifier, but the general indication of certain biomarkers remains fixed, i.e. high values or “relative over-expression” always contributes to either a responsiveness (genes with a positive weight) or resistance (genes with a negative weight). Therefore, in the context of the overall gene expression classifier, relative expression can indicate if either up- or down-regulation of a certain biomarker is indicative of responsiveness or resistance to a therapeutic agent.

In one embodiment, the biomarker expression profile of a test sample, for example a patient tissue sample, is evaluated by a linear classifier. As used herein, a linear classifier refers to a weighted sum of the individual biomarker intensities into a compound decision score (“decision function”). The decision score is then compared to a pre-defined cut-off score threshold, corresponding to a certain set-point in terms of sensitivity and specificity which indicates if a sample is above the score threshold (decision function positive) or below (decision function negative).

Effectively, this means that the data space, i.e. the set of all possible combinations of biomarker expression values, is split into two mutually exclusive halves corresponding to different clinical classifications or predictions, e.g. one corresponding to responsiveness to a therapeutic agent and the other to resistance. In the context of the overall classifier, relative over-expression of a certain biomarker can either increase the decision score (positive weight) or reduce it (negative weight) and thus contribute to an overall decision of, for example, responsiveness or resistance to a therapeutic agent.

The term “area under the curve” or “AUC” refers to the area under the curve of a receiver operating characteristic (ROC) curve, both of which are well known in the art. AUC measures are useful for comparing the accuracy of a classifier across the complete data range. Classifiers with a greater AUC have a greater capacity to classify unknowns correctly between two groups of interest (e.g., OAC cancer samples and normal or control samples). ROC curves are useful for plotting the performance of a particular feature (e.g., any of the biomarkers described herein and/or any item of additional biomedical information) in distinguishing between two populations (e.g., individuals responding and not responding to a therapeutic agent). Typically, the feature data across the entire population (e.g., the cases and controls) are sorted in ascending order based on the value of a single feature. Then, for each value for that feature, the true positive and false positive rates for the data are calculated. The true positive rate is determined by counting the number of cases above the value for that feature and then dividing by the total number of cases. The false positive rate is determined by counting the number of controls above the value for that feature and then dividing by the total number of controls. Although this definition refers to scenarios in which a feature is elevated in cases compared to controls, this definition also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to provide a single sum value, and this single sum value can be plotted in a ROC curve. Additionally, any combination of multiple features, in which the combination derives a single output value, can be plotted in a ROC curve. These combinations of features may comprise a test. The ROC curve is the plot of the true positive rate (sensitivity) of a test against the false positive rate (1-specificity) of the test.

The interpretation of this quantity, i.e. the cut-off threshold responsiveness or resistance to a therapeutic agent, is derived in the development phase (“training”) from a set of patients with known outcome. The corresponding weights and the responsiveness/resistance cut-off threshold for the decision score are fixed a priori from training data by methods known to those skilled in the art. In a preferred embodiment of the present method, Partial Least Squares Discriminant Analysis (PLS-DA) is used for determining the weights. (L. Ståhle, S. Wold, J. Chemom. 1 (1987) 185-196; D. V. Nguyen, D. M. Rocke, Bioinformatics 18 (2002) 39-50). Other methods for performing the classification, known to those skilled in the art, may also be used with the methods described herein, for example when applied to the transcripts of an oesophageal cancer (OAC) classifier.

Different methods can be used to convert quantitative data measured on these biomarkers into a prognosis or other predictive use. These methods include, but are not limited to methods from the fields of pattern recognition (Duda et al. Pattern Classification, 2^{nd} ed., John Wiley, New York 2001), machine learning (Schölkopf et al. Learning with Kernels, MIT Press, Cambridge 2002, Bishop, Neural Networks for Pattern Recognition, Clarendon Press, Oxford 1995), statistics (Hastie et al. The Elements of Statistical Learning, Springer, New York 2001), bioinformatics (Dudoit et al., 2002, J. Am. Statist. Assoc. 97:77-87, Tibshirani et al., 2002, Proc. Natl. Acad. Sci. USA 99:6567-6572) or chemometrics (Vandeginste, et al., Handbook of Chemometrics and Qualimetrics, Part B, Elsevier, Amsterdam 1998).

In a training step, a set of patient samples for both responsiveness/resistance cases are measured and the prediction method is optimised using the inherent information from this training data to optimally predict the training set or a future sample set. In this training step, the used method is trained or parameterised to predict from a specific intensity pattern to a specific predictive call. Suitable transformation or pre-processing steps might be performed with the measured data before it is subjected to the prognostic method or algorithm.

In a preferred embodiment of the invention, a weighted sum of the pre-processed intensity values for each transcript is formed and compared with a threshold value optimised on the training set (Duda et al. Pattern Classification, 2^{nd} ed., John Wiley, New York 2001). The weights can be derived by a multitude of linear classification methods, including but not limited to Partial Least Squares (PLS, (Nguyen et al., 2002, Bioinformatics 18 (2002) 39-50)) or Support Vector Machines (SVM, (Schölkopf et al. Learning with Kernels, MIT Press, Cambridge 2002)).

In another embodiment of the invention, the data is transformed non-linearly before applying a weighted sum as described above. This non-linear transformation might include increasing the dimensionality of the data. The non-linear transformation and weighted summation might also be performed implicitly, e.g. through the use of a kernel function. (Schölkopf et al. Learning with Kernels, MIT Press, Cambridge 2002).

In another embodiment of the invention, a new data sample is compared with two or more class prototypes, being either real measured training samples or artificially created prototypes. This comparison is performed using suitable similarity measures, for example, but not limited to Euclidean distance (Duda et al. Pattern Classification, 2^{nd} ed., John Wiley, New York 2001), correlation coefficient (Van't Veer, et al. 2002, Nature 415:530) etc. A new sample is then assigned to the prognostic group with the closest prototype or the highest number of prototypes in the vicinity.

In another embodiment of the invention, decision trees (Hastie et al., The Elements of Statistical Learning, Springer, New York 2001) or random forests (Breiman, Random Forests, Machine Learning 45:5 2001) are used to make a prognostic call from the measured intensity data for the transcript set or their products.

In another embodiment of the invention neural networks (Bishop, Neural Networks for Pattern Recognition, Clarendon Press, Oxford 1995) are used to make a prognostic call from the measured intensity data for the transcript set or their products.

In another embodiment of the invention, discriminant analysis (Duda et al., Pattern Classification, 2^{nd} ed., John Wiley, New York 2001), comprising but not limited to linear, diagonal linear, quadratic and logistic discriminant analysis, is used to make a prognostic call from the measured intensity data for the transcript set or their products.

In another embodiment of the invention, Prediction Analysis for Microarrays (PAM, (Tibshirani et al., 2002, Proc. Natl. Acad. Sci. USA 99:6567-6572)) is used to make a prognostic call from the measured intensity data for the transcript set or their products.

In another embodiment of the invention, Soft Independent Modelling of Class Analogy (SIMCA, (Wold, 1976, Pattern Recogn. 8:127-139)) is used to make a predictive call from the measured intensity data for the transcript set or their products.

In another embodiment of the invention, c-index is used to quantify predictive ability. This index applies biomarkers to a continuous response variable that can be censored. The c index is the proportion of all pairs of subjects whose survival times can be ordered such that the subject with the higher predicted survival is the one who survived longer. Two subject's survival times cannot be ordered if both subjects are censored or if one has failed and the follow up time of the other is less than the failure time of the first. The c index is the probability of concordance between predicted and observed survival, with c=0.5 for random prediction and c=1 for a perfectly discriminating model. (Frank E. Harrell, Jr. Regression Modeling Strategies, 2001).

### Therapeutic Agents

As described above, the methods described herein permit the classification of a patient suffering from OAC, including early stage OAC as responsive or non-responsive to a therapeutic agent that targets tumors with abnormal DNA repair (hereinafter referred to as a “DNA-damaging therapeutic agent”). As used herein “DNA-damaging therapeutic agent” includes agents known to damage DNA directly, agents that prevent DNA damage repair, agents that inhibit DNA damage signaling, agents that inhibit DNA damage induced cell cycle arrest, and agents that inhibit processes indirectly leading to DNA damage. Some current such therapeutics used to treat OAC include, but are not limited to, the following DNA-damaging therapeutic agents.

1) DNA Damaging Agents:
a. Alkylating agents (platinum containing agents such as cisplatin, carboplatin, and oxaliplatin; cyclophosphamide; busulphan).
b. Topoisomerase I inhibitors (irinotecan;topotecan)
c. Topoisomerase II inhibitors (etoposide;anthracyclines such as doxorubicin and epirubicin)
d. Ionising radiation

2) DNA Repair Targeted Therapies
a. Inhibitors of Non-homologous end-joining (DNA-PK inhibitors, Nu7441, NU7026)
b. Inhibitors of homologous recombination
c. Inhibitors of nucleotide excision repair
d. Inhibitors of base excision repair (PARP inhibitors, AG014699, AZD2281, ABT-888, MK4827, BSI-201, INO-1001, TRC-102, APEX 1 inhibitors, APEX 2 inhibitors, Ligase III inhibitors
e. Inhibitors of the Fanconi anemia pathway

3) Inhibitors of DNA Damage Signalling
a. ATM inhibitors (CP466722)
b. CHK 1 inhibitors (XL-844,UCN-01, AZD7762, PF00477736)
c. CHK 2 inhibitors (XL-844, AZD7762, PF00477736)
d. ATR inhibitors (AZ20)

4) Inhibitors of DNA Damage Induced Cell Cycle Arrest
a. Wee1 kinase inhibitors
b. CDC25a, b or c inhibitors

5) Inhibition of Processes Indirectly Leading to DNA Damage
a. Histone deacetylase inhibitors
b. Heat shock protein inhibitors (geldanamycin, AUY922),

6) Inhibitors of DNA Synthesis:
a. Pyrimidine analogues (5-FU, gemcitabine)
b. Prodrugs (capecitabine)

As discussed above the therapeutic agents for which responsiveness is predicted may be applied in a neoadjuvant setting. However, they may be utilised in an adjuvant setting additionally or alternatively.

The invention described herein is not limited to any one DNA-damaging therapeutic agent; it can be used to identify responders and non-responders to any of a range of DNA-damaging therapeutic agent, for example those that directly or indirectly affect DNA damage and/or DNA damage repair. In some embodiments, the DNA-damaging therapeutic agent comprises one or more substances selected from the group consisting of: a DNA damaging agent, a DNA repair targeted therapy, an inhibitor of DNA damage signalling, an inhibitor of DNA damage induced cell cycle arrest, a histone deacetylase inhibitor, a heat shock protein inhibitor and an inhibitor of DNA synthesis. More specifically, the DNA-damaging therapeutic agent may be selected from one or more of a platinum-containing agent, a nucleoside analogue such as gemcitabine or 5-fluorouracil or a prodrug thereof such as capecitabine, an anthracycline such as epirubicin or doxorubicin, an alkylating agent such as cyclophosphamide, an ionising radiation or a combination of (ionising) radiation and chemotherapy (chemoradiation). In particular embodiments, the DNA-damaging therapeutic agent comprises a platinum-containing agent, such as a platinum based agent selected from cisplatin, carboplatin and oxaliplatin. The methods and kits may predict responsiveness to treatment with the DNA-damaging therapeutic agent together with a further therapy (such as radiation) or drug. Thus, the methods and kits may predict responsiveness to a combination therapy. In some embodiments, the further drug is a mitotic inhibitor. The mitotic inhibitor may be a vinca alkaloid or a taxane. In specific embodiments, the vinca alkaloid is vinorelbine. In other embodiments, the taxane is paclitaxel or docetaxel. In certain embodiments, responders to the following treatments are identified: cisplatin/carboplatin and 5-fluorouracil (CF), cisplatin/carboplatin and capecitabine (CX), epirubicin/doxorubicin, cisplatin/carboplatin and fluorouracil (ECF), epirubicin/doxorubicin, oxaliplatin and capecitabine (EOX), and combinations with paclitaxel, irinotecan and vinorelbine, radiation or chemoradiation with or without taxanes, in treatment of OAC.

### Diseases and Tissue Sources

The predictive classifiers described herein are useful for determining responsiveness or resistance to a therapeutic agent for treating oesophageal cancer, in particular OAC. The oesophageal cancer is typically oesophageal adenocarcinoma (OAC) and may be early stage.

In one embodiment, the methods described herein refer to OACs that are treated with chemotherapeutic agents of the classes DNA damaging agents, DNA repair target therapies, inhibitors of DNA damage signalling, inhibitors of DNA damage induced cell cycle arrest, inhibition of processes indirectly leading to DNA damage and inhibition of DNA synthesis, but not limited to these classes. Each of these chemotherapeutic agents is considered a “DNA-damaging therapeutic agent” as the term is used herein.

“Biological sample”, “sample”, and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, and serum), sputum, tears, mucus, nasal washes, nasal aspirate, breath, urine, semen, saliva, meningeal fluid, amniotic fluid, glandular fluid, lymph fluid, nipple aspirate, bronchial aspirate, synovial fluid, joint aspirate, ascites, cells, a cellular extract, and cerebrospinal fluid. This also includes experimentally separated fractions of all of the preceding. For example, a blood sample can be fractionated into serum or into fractions containing particular types of blood cells, such as red blood cells or white blood cells (leukocytes). If desired, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Samples may be obtained by endoscopy in some embodiments. A “biological sample” obtained or derived from an individual includes any such sample that has been processed in any suitable manner after being obtained from the individual.

In such cases, the target cells may be tumor cells, for example OAC cells. The target cells are derived from any tissue source, including human and animal tissue, such as, but not limited to, a newly obtained sample, a frozen sample, a biopsy sample, a sample of bodily fluid, a blood sample, preserved tissue such as a paraffin-embedded fixed tissue sample (i.e., a tissue block), or cell culture.

In some specific embodiments, the samples may or may not comprise vesicles.

### Methods and Kits

### Kits for Gene Expression Analysis

Reagents, tools, and/or instructions for performing the methods described herein can be provided in a kit. For example, the kit can contain reagents, tools, and instructions for determining an appropriate therapy for a oesophageal cancer patient. Such a kit can include reagents for collecting a tissue sample from a patient, such as by biopsy, and reagents for processing the tissue. The kit can also include one or more reagents for performing a biomarker expression analysis, such as reagents for performing nucleic acid amplification, including RT-PCR and qPCR, NGS, northern blot, proteomic analysis, or immunohistochemistry to determine expression levels of biomarkers in a sample of a patient. For example, primers for performing RT-PCR, probes for performing northern blot analyses, and/or antibodies for performing proteomic analysis such as Western blot, immunohistochemistry and ELISA analyses can be included in such kits. Appropriate buffers for the assays can also be included. Detection reagents required for any of these assays can also be included. The appropriate reagents and methods are described in further detail herein.

In certain embodiments, the target sequences listed in Tables 1A (SEQ ID NO: 1-24), 1B (SEQ ID NO: 25-50) and/or 3 (SEQ ID NO: 51-230), or subsets thereof, may be used in the methods and kits described herein. The target sequences may be utilised for the purposes of designing primers and/or probes which hybridize to the target sequences. Design of suitable primers and/or probes is within the capability of one skilled in the art once the target sequence is identified. Various primer design tools are freely available to assist in this process such as the NCBI Primer-BLAST tool. The primers and/or probes may be designed such that they hybridize to the target sequence under stringent conditions. Primers and/or probes may be at least 15, 16, 17, 18, 19, 20, 21, 22, 23, 24 or 25 (or more) nucleotides in length. It should be understood that each subset can include multiple primers and/or probes directed to the same biomarker. The tables show in some cases multiple target sequences within the same overall gene. Such primers and/or probes may be included in kits useful for performing the methods of the invention. The kits may be array or PCR based kits for example and may include additional reagents, such as a polymerase and/or dNTPs for example. The kits featured herein can also include an instruction sheet describing how to perform the assays for measuring biomarker expression. The instruction sheet can also include instructions for how to determine a reference cohort, including how to determine expression levels of biomarkers in the reference cohort and how to assemble the expression data to establish a reference for comparison to a test patient. The instruction sheet can also include instructions for assaying biomarker expression in a test patient and for comparing the expression level with the expression in the reference cohort to subsequently determine the appropriate chemotherapy for the test patient. Methods for determining the appropriate chemotherapy are described above and can be described in detail in the instruction sheet.

Informational material included in the kits can be descriptive, instructional, marketing or other material that relates to the methods described herein and/or the use of the reagents for the methods described herein. For example, the informational material of the kit can contain contact information, e.g., a physical address, email address, website, or telephone number, where a user of the kit can obtain substantive information about performing a gene expression analysis and interpreting the results, particularly as they apply to a human's likelihood of having a positive response to a specific therapeutic agent.

The kits featured herein can also contain software necessary to infer a patient's likelihood of having a positive response to a specific therapeutic agent from the biomarker expression.

The kits may, in some embodiments, additionally contain the DNA-damaging therapeutic agent for administration in the event that the individual is predicted to be responsive. Any of the specific agents or combinations of agents described herein to treat OAC may be incorporated into the kits. The agent or combination of agents may be provided in a form, such as a dosage form, that is tailored to OAC treatment specifically. The kit may be provided with suitable instructions for administration according to OAC treatment regimens, for example in the context of neoadjuvant and/or adjuvant treatment.

### a) Gene Expression Profiling Methods

Measuring mRNA in a biological sample may be used as a surrogate for detection of the level of the corresponding protein in the biological sample. Thus, any of the biomarkers or biomarker panels described herein can also be detected by detecting the appropriate RNA. Methods of gene expression profiling include, but are not limited to, microarray, RT-PCT, qPCR, NGS, northern blots, SAGE, mass spectrometry.

mRNA expression levels may be measured by reverse transcription quantitative polymerase chain reaction (RT-PCR followed with qPCR). RT-PCR is used to create a cDNA from the mRNA. The cDNA may be used in a qPCR assay to produce fluorescence as the DNA amplification process progresses. By comparison to a standard curve, qPCR can produce an absolute measurement such as number of copies of mRNA per cell. Northern blots, microarrays, Invader assays, and RT-PCR combined with capillary electrophoresis have all been used to measure expression levels of mRNA in a sample. See Gene Expression Profiling: Methods and Protocols, Richard A. Shimkets, editor, Humana Press, 2004.

miRNA molecules are small RNAs that are non-coding but may regulate gene expression. Any of the methods suited to the measurement of mRNA expression levels can also be used for the corresponding miRNA. Recently many laboratories have investigated the use of miRNAs as biomarkers for disease. Many diseases involve widespread transcriptional regulation, and it is not surprising that miRNAs might find a role as biomarkers. The connection between miRNA concentrations and disease is often even less clear than the connections between protein levels and disease, yet the value of miRNA biomarkers might be substantial. Of course, as with any RNA expressed differentially during disease, the problems facing the development of an in vitro diagnostic product will include the requirement that the miRNAs survive in the diseased cell and are easily extracted for analysis, or that the miRNAs are released into blood or other matrices where they must survive long enough to be measured. Protein biomarkers have similar requirements, although many potential protein biomarkers are secreted intentionally at the site of pathology and function, during disease, in a paracrine fashion. Many potential protein biomarkers are designed to function outside the cells within which those proteins are synthesized.

Gene expression may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

The foregoing assays enable the detection of biomarker values that are useful in methods for predicting responsiveness of a cancer therapeutic agent, where the methods comprise detecting, in a biological sample from an individual suffering from OAC, at least N biomarker values that each correspond to a biomarker selected from the group consisting of the biomarkers provided in Tables 1 to 4, wherein a classification, as described in detail herein, using the biomarker values indicates whether the individual will be responsive to a therapeutic agent. While certain of the described predictive biomarkers are useful alone for predicting responsiveness to a therapeutic agent, methods are also described herein for the grouping of multiple subsets of the biomarkers that are each useful as a panel of two or more biomarkers. Thus, various embodiments of the instant application provide combinations comprising N biomarkers, wherein N is at least three biomarkers. It will be appreciated that N can be selected to be any number from any of the above-described ranges, as well as similar, but higher order, ranges. In accordance with any of the methods described herein, biomarker values can be detected and classified individually or they can be detected and classified collectively, as for example in a multiplex assay format.

### b) Microarray Methods

In one embodiment, the present invention makes use of “oligonucleotide arrays” (also called herein “microarrays”). Microarrays can be employed for analyzing the expression of biomarkers in a cell, and especially for measuring the expression of biomarkers of cancer tissues.

In one embodiment, biomarker arrays are produced by hybridizing detectably labeled polynucleotides representing the mRNA transcripts present in a cell (e.g., fluorescently-labeled cDNA synthesized from total cell mRNA or labeled cRNA) to a microarray. A microarray is a surface with an ordered array of binding (e.g., hybridization) sites for products of many of the genes in the genome of a cell or organism, preferably most or almost all of the genes. Microarrays can be made in a number of ways known in the art. However produced, microarrays share certain characteristics. The arrays are reproducible, allowing multiple copies of a given array to be produced and easily compared with each other. Preferably the microarrays are small, usually smaller than 5 cm², and they are made from materials that are stable under binding (e.g., nucleic acid hybridization) conditions. A given binding site or unique set of binding sites in the microarray will specifically bind the product of a single gene in the cell. In a specific embodiment, positionally addressable arrays containing affixed nucleic acids of known sequence at each location are used.

It will be appreciated that when cDNA complementary to the RNA of a cell is made and hybridized to a microarray under suitable hybridization conditions, the level of hybridization to the site in the array corresponding to any particular gene will reflect the prevalence in the cell of mRNA transcribed from that gene/biomarker. For example, when detectably labeled (e.g., with a fluorophore) cDNA or cRNA complementary to the total cellular mRNA is hybridized to a microarray, the site on the array corresponding to a gene (i.e., capable of specifically binding the product of the gene) that is not transcribed in the cell will have little or no signal (e.g., fluorescent signal), and a gene for which the encoded mRNA is prevalent will have a relatively strong signal. Nucleic acid hybridization and wash conditions are chosen so that the probe “specifically binds” or “specifically hybridizes’ to a specific array site, i.e., the probe hybridizes, duplexes or binds to a sequence array site with a complementary nucleic acid sequence but does not hybridize to a site with a non-complementary nucleic acid sequence. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can be demonstrated that specific hybridization conditions result in specific hybridization by carrying out a hybridization assay including negative controls using routine experimentation.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65° C. for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif. (1992).

Microarray platforms include those manufactured by companies such as Affymetrix, Illumina and Agilent. Examples of microarray platforms manufactured by Affymetrix include the U133 Plus2 array, the Almac proprietary Xcel™ array and the Almac proprietary Cancer DSAs®, including the Breast Cancer DSA® and Lung Cancer DSA®

### c) Immunoassay Methods

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Clinical Uses

In some embodiments, methods are provided for identifying and/or selecting an OAC patient who is responsive to a therapeutic regimen. In particular, the methods are directed to identifying or selecting a cancer patient who is responsive to a therapeutic regimen that includes administering an agent that directly or indirectly damages DNA. Methods are also provided for identifying a patient who is non-responsive to a therapeutic regimen. These methods typically include determining the level of expression of a collection of predictive markers in a patient's tumor (primary, metastatic or other derivatives from the tumor such as, but not limited to, blood, or components in blood, urine, saliva and other bodily fluids)(e.g., a patient's cancer cells), comparing the level of expression to a reference expression level, and identifying whether expression in the sample includes a pattern or profile of expression of a selected predictive biomarker or biomarker set which corresponds to response or non-response to therapeutic agent.

In some embodiments a method of predicting responsiveness of an individual having oesophageal adenocarcinoma (OAC) to treatment with a DNA-damaging therapeutic agent comprises:
a. measuring expression levels of one or more biomarkers in a test sample obtained from the individual, wherein the one or more biomarkers are selected from Table 1A and/or 1B and/or 2A and/or 2B and/or 3 and/or 4;
b. deriving a test score that captures the expression levels;
c. providing a threshold score comprising information correlating the test score and responsiveness;
d. and comparing the test score to the threshold score; wherein responsiveness is predicted when the test score exceeds the threshold score.

In specific embodiments, a method of predicting responsiveness of an individual having oesophageal adenocarcinoma (OAC) to treatment with a DNA-damaging therapeutic agent comprises the following steps: obtaining a test sample from the individual; measuring expression levels of one or more biomarkers in the test sample, wherein the one or more biomarkers are selected from the group consisting of CXCL10, MX1, IDO1, IF144L, CD2, GBP5, PRAME, ITGAL, LRP4, and APOL3; deriving a test score that captures the expression levels; providing a threshold score comprising information correlating the test score and responsiveness; and comparing the test score to the threshold score; wherein responsiveness is predicted when the test score exceeds the threshold score. One of ordinary skill in the art can determine an appropriate threshold score, and appropriate biomarker weightings and bias, using the teachings provided herein including the teachings of Example 1.

In other embodiments, the method of predicting responsiveness of an individual having oesophageal adenocarcinoma (OAC) to treatment with to a DNA-damaging therapeutic agent comprises measuring the expression levels of one or more biomarkers in the test sample, wherein the one or more biomarkers are selected from the group consisting of CXCL10, MX1, IDO1, IF144L, CD2, GBP5, PRAME, ITGAL, LRP4, APOL3, CDR1, FYB, TSPAN7, RAC2, KLHDC7B, GRB14, AC138128.1, KIF26A, CD274, CD109, ETV7, MFAP5, OLFM4, PI15, FOSB, FAM19A5, NLRC5, PRICKLE1, EGR1, CLDN10, ADAMTS4, SP140L, ANXA1, RSAD2, ESR1, IKZF3, OR2I1P, EGFR, NAT1, LATS2, CYP2B6, PTPRC, PPP1R1A, and AL137218.1. Tables 2A and 2B provide exemplary gene signatures (or gene classifiers) wherein the biomarkers consist of 40 or 44 of the gene products listed therein, respectively, and wherein a threshold score is derived from the individual gene product weightings listed therein. In one of these embodiments wherein the biomarkers consist of the 44 gene products listed in Table 2B, and the biomarkers are associated with the weightings provided in Table 2B, a test score that exceeds a threshold score, such as a threshold score of 0.3681 indicates a likelihood that the individual will be responsive to a DNA-damaging therapeutic agent.

A cancer is “responsive” to a therapeutic agent if its rate of growth is inhibited as a result of contact with the therapeutic agent, compared to its growth in the absence of contact with the therapeutic agent. Growth of a cancer can be measured in a variety of ways, for instance, the size of a tumor or the expression of tumor markers appropriate for that tumor type may be measured.

A cancer is “non-responsive” to a therapeutic agent if its rate of growth is not inhibited, or inhibited to a very low degree, as a result of contact with the therapeutic agent when compared to its growth in the absence of contact with the therapeutic agent. As stated above, growth of a cancer can be measured in a variety of ways, for instance, the size of a tumor or the expression of tumor markers appropriate for that tumor type may be measured. The quality of being non-responsive to a therapeutic agent is a highly variable one, with different cancers exhibiting different levels of “non-responsiveness” to a given therapeutic agent, under different conditions. Still further, measures of non-responsiveness can be assessed using additional criteria beyond growth size of a tumor, including patient quality of life, degree of metastases, etc.

An application of this test will predict end points including, but not limited to, overall survival, progression free survival, radiological response, as defined by RECIST, complete response, partial response, stable disease and serological markers such as, but not limited to, PSA, CEA, CA125, CA15-3 and CA19-9. In other embodiments of this invention it can be used to evaluate endoscopic ultrasound, CT, spiral CT, FDG-PET, pathologic, histological response in oesophageal cancer treated with DNA damaging combination therapies, alone or in the context of standard treatment.

Array or non-array based methods for detection, quantification and qualification of RNA, DNA or protein within a sample of one or more nucleic acids or their biological derivatives such as encoded proteins may be employed, including quantitative PCR (QPCR), enzyme-linked immunosorbent assay (ELISA) or immunohistochemistry (IHC) and the like.

After obtaining an expression profile from a sample being assayed, the expression profile is compared with a reference or control profile to make a diagnosis regarding the therapy responsive phenotype of the cell or tissue, and therefore host, from which the sample was obtained. The terms “reference” and “control” as used herein in relation to an expression profile mean a standardized pattern of gene or gene product expression or levels of expression of certain biomarkers to be used to interpret the expression classifier of a given patient and assign a prognostic or predictive class. The reference or control expression profile may be a profile that is obtained from a sample known to have the desired phenotype, e.g., responsive phenotype, and therefore may be a positive reference or control profile. In addition, the reference profile may be from a sample known to not have the desired phenotype, and therefore be a negative reference profile.

If quantitative PCR is employed as the method of quantitating the levels of one or more nucleic acids, this method may quantify the PCR product accumulation through measurement of fluorescence released by a dual-labeled fluorogenic probe (e.g. a TaqMan® probe or a molecular beacon or FRET/Light Cycler probes). Some methods may not require a separate probe, such as the Scorpion and Ampliflyor systems where the probes are built into the primers.

In certain embodiments, the obtained expression profile is compared to a single reference profile to obtain information regarding the phenotype of the sample being assayed. In yet other embodiments, the obtained expression profile is compared to two or more different reference profiles to obtain more in depth information regarding the phenotype of the assayed sample. For example, the obtained expression profile may be compared to a positive and negative reference profile to obtain confirmed information regarding whether the sample has the phenotype of interest.

The comparison of the obtained expression profile and the one or more reference profiles may be performed using any convenient methodology, where a variety of methodologies are known to those of skill in the array art, e.g., by comparing digital images of the expression profiles, by comparing databases of expression data, etc. Patents describing ways of comparing expression profiles include, but are not limited to, U.S. Pat. Nos. 6,308,170 and 6,228,575, the disclosures of which are herein incorporated by reference. Methods of comparing expression profiles are also described above.

The comparison step results in information regarding how similar or dissimilar the obtained expression profile is to the one or more reference profiles, which similarity information is employed to determine the phenotype of the sample being assayed. For example, similarity with a positive control indicates that the assayed sample has a responsive phenotype similar to the responsive reference sample. Likewise, similarity with a negative control indicates that the assayed sample has a non-responsive phenotype to the non-responsive reference sample.

The level of expression of a biomarker can be further compared to different reference expression levels. For example, a reference expression level can be a predetermined standard reference level of expression in order to evaluate if expression of a biomarker or biomarker set is informative and make an assessment for determining whether the patient is responsive or non-responsive. Additionally, determining the level of expression of a biomarker can be compared to an internal reference marker level of expression which is measured at the same time as the biomarker in order to make an assessment for determining whether the patient is responsive or non-responsive. For example, expression of a distinct marker panel which is not comprised of biomarkers of the invention, but which is known to demonstrate a constant expression level can be assessed as an internal reference marker level, and the level of the biomarker expression is determined as compared to the reference. In an alternative example, expression of the selected biomarkers in a tissue sample which is a non-tumor sample can be assessed as an internal reference marker level. The level of expression of a biomarker may be determined as having increased expression in certain aspects. The level of expression of a biomarker may be determined as having decreased expression in other aspects. The level of expression may be determined as no informative change in expression as compared to a reference level. In still other aspects, the level of expression is determined against a pre-determined standard expression level as determined by the methods provided herein.

The invention is also related to guiding conventional treatment of patients. Patients in which the diagnostics test reveals that they are responders to the drugs, of the classes that directly or indirectly affect DNA damage and/or DNA damage repair, can be administered with that therapy and both patient and oncologist can be confident that the patient will benefit. Patients that are designated non-responders by the diagnostic test can be identified for alternative therapies which are more likely to offer benefit to them.

The invention further relates to selecting patients for clinical trials where novel drugs of the classes that directly or indirectly affect DNA damage and/or DNA damage repair in order to treat OAC. Enrichment of trial populations with potential responders will facilitate a more thorough evaluation of that drug under relevant criteria.

The invention still further relates to methods of diagnosing patients as having or being susceptible to developing OAC associated with a DNA damage response deficiency (DDRD). DDRD is defined herein as any condition wherein a cell or cells of the patient have a reduced ability to repair DNA damage, which reduced ability is a causative factor in the development or growth of a tumor. The DDRD diagnosis may be associated with a mutation in the Fanconi anemia/BRCA pathway. The DDRD diagnosis may also be associated with adenocarcinoma. The methods of diagnosing an individual having oesophageal adenocarcinoma (OAC) may comprise:
a. measuring expression levels of one or more biomarkers in a test sample obtained from the individual, wherein the one or more biomarkers are selected from Table 1A, 1B, 2A, 2B, 3 or 4;
b. deriving a test score that captures the expression levels;
c. providing a threshold score comprising information correlating the test score and diagnosis of OAC;
d. and comparing the test score to the threshold score; wherein the individual is determined to have OAC or be susceptible to developing OAC when the test score exceeds the threshold score.

The methods of diagnosis may comprise the steps of obtaining a test sample from the individual; measuring expression levels of one or more biomarkers in the test sample, wherein the one or more biomarkers are selected from the group consisting of CXCL10, MX1, IDO1, IF144L, CD2, GBP5, PRAME, ITGAL, LRP4, and APOL3; deriving a test score that captures the expression levels; providing a threshold score comprising information correlating the test score and a diagnosis of the OAC; and comparing the test score to the threshold score; wherein the individual is determined to have the cancer or is susceptible to developing the cancer when the test score exceeds the threshold score. One of ordinary skill in the art can determine an appropriate threshold score, and appropriate biomarker weightings, using the teachings provided herein including the teachings of Example 1.

In other embodiments, the methods of diagnosing patients as having or being susceptible to developing OAC associated with DDRD comprise measuring expression levels of one or more biomarkers in the test sample, wherein the one or more biomarkers are selected from the group consisting of CXCL10, MX1, IDO1, IF144L, CD2, GBP5, PRAME, ITGAL, LRP4, APOL3, CDR1, FYB, TSPAN7, RAC2, KLHDC7B, GRB14, AC138128.1, KIF26A, CD274, CD109, ETV7, MFAP5, OLFM4, PI15, FOSB, FAM19A5, NLRC5, PRICKLE1, EGR1, CLDN10, ADAMTS4, SP140L, ANXA1, RSAD2, ESR1, IKZF3, OR2I1P, EGFR, NAT1, LATS2, CYP2B6, PTPRC, PPP1R1A, and AL137218.1. Tables 2A and 2B provide exemplary gene signatures (or gene classifiers) wherein the biomarkers consist of 40 or 44 of the gene products listed therein, respectively, and wherein a threshold score is derived from the individual gene product weightings listed therein. In one of these embodiments wherein the biomarkers consist of the 44 gene products listed in Table 2B, and the biomarkers are associated with the weightings provided in Table 2B, a test score that exceeds a threshold score, such as a threshold score of 0.3681, indicates a diagnosis of OAC or of being susceptible to developing OAC.

### DESCRIPTION OF THE FIGURES

FIG. 1: Kaplan Meier plot for DDRD signature predicting relapse-free survival (RFS) in EAC patients treated with neo-adjuvant chemotherapy.

FIG. 2: Kaplan Meier for DDRD signature predicting overall survival (PFS) in EAC patients treated with neo-adjuvant chemotherapy.

FIG. 3: Kaplan Meier for DDRD signature predicting survival (PFS) in EAC patients not treated with neo-adjuvant chemotherapy.

The following examples are offered by way of illustration and not by way of limitation.

### EXAMPLES

### Example 1

### Application of DDRD Assay to Oesophageal Adenocarcinoma (OAC) and Validation

### Methods

### Samples

46 FFPE pre-chemotherapy (platinum-based; cisplatin treatment) oesophageal endoscopy biopsy samples with appropriate ethical approval and consent information for the use of human tissue were used to demonstrate the predictive ability of the DDRD assay (based on the 44 genes listed in table 2B) in Oesophageal cancer (OAC). Five FFPE tissue slides were received per sample along with a “marked up” Haematoxylin & Eosin (H&E) stained slide per sample. H&E slides were reviewed for pathology subtypes and marked for macrodissection.

### Gene Expression Profiling

All samples were processed in a quality controlled environment according to the appropriate Standard Operating Procedures (SOPs).

All 5 sections per sample were macrodissected, and extracted using the Ambion RecoverAll kit. The lysate was split in two and for most cases only one half of the material was sufficient for profiling. In 2 cases both aliquots of the macrodissected material was extracted Samples were randomised and extracted over 3 batches.

Following extraction sample concentration was determined using the Nanodrop 1000 spectrophotometer. Vacuum concentration was required for 5 samples to yield a suitable concentration of RNA for cDNA amplification.

All 36 RNA samples, whether these were the original primary extract, vacuum concentrated or the secondary re-extract, advanced to amplification using the NUGEN V3 FFPE Amplification kit. The 46 samples were profiled using the Ovation® FFPE WTA System. Following quality control of the generated cDNA using both the spectrophotometer and the Agilent 2100 Bioanalyzer, the samples were fragmented and labelled using the NuGEN Encore Biotin module. Following the fragmentation QC, the fragmented and labelled product was then hybridized to Xcel arrays in accordance with the NuGEN guidelines for hybridisation onto Affymetrix GeneChip® arrays.

### Array QC

Following microarray profiling a quality control (QC) assessment of the data was performed. The sample and array quality was assessed using Almac's proprietary toolbox. One of the forty-six samples failed microarray QC. This sample failed on image analysis, present call, scaling factor and distribution. Therefore 45 samples were used in subsequent analysis.

### Data Preparation

Samples were pre-processed using Almac's pre-processing toolbox, with RMA settings (background correction; quantile normalisation using the median for average; and median polish summarisation). A cross mapping of probesets representing the DDRD genes was used to extract relevant probesets for analysis.

### Calculating Classifier Scores

The probe sets that map to the genes in the classifier are determined, excluding anti-sense probe sets (if applicable). The median intensity over all probe sets relating to each gene in the classifier is calculated resulting in a reduced gene intensity matrix. If no probe sets exist for the gene on the particular array platform, the observed average from the training data will be used as a replacement. The median value of all probe sets for each sample is calculated and subtracted from the reduced gene intensity matrix.

The classifier score of a sample is calculated as a weighted sum of the expression of genes in the signature.

$$SignatureScore = {\sum }_{i} {w}_{i} \times ( {ge}_{i} - {b}_{i} - {a}_{median} ) + k$$

where wᵢ is a weight for each gene, bᵢ is a gene-specific bias which is required to mean center each gene relative to the training data (Table 4), a_{median} is the median expression intensity across all probe sets on the array, geᵢ is the observed gene expression level after pre-processing, and k=0.3738 is a constant offset.

The classifier score is calculated for each sample, which can then be used to stratify patients from, for example, more likely to respond to less likely to respond.

### Results

TABLE 5
Multivariable analysis of recurrence free survival
(43 samples, 20 non-events and 23 events)

| | Hazard | Lower | Upper | |
|---|---|---|---|---|
| | Ratio | 95% CI | 95% CI | P-value |
| DDRD-positive | 0.069 | 0.007 | 0.613 | 0.0189 |
| N Stage 1 vs 0 | 3.173 | 0.933 | 10.783 | 0.0644 |
| N Stage 2&3 vs 0 | 7.089 | 2.092 | 24.023 | 0.0017 |
| Surgical T Stage 2 vs 0&1 | 0.249 | 0.020 | 3.161 | 0.2838 |
| Surgical T Stage 3+ vs 0&1 | 0.995 | 0.120 | 8.273 | 0.9962 |

Using Cox proportional hazards regression, there is a statistically significant effect of the DDRD assay on recurrence free survival. The effect is statistically significant when other important risk factors are included in the model (hazard ratio 0.07, 95% CI (0.007, 0.643), p=0.019). Covariates included in the model were directed by clinical expertise.

TABLE 6
Multivariable analysis of overall survival
(43 samples, 17 non-events and 26 events)

| | Hazard | Lower | Upper | |
|---|---|---|---|---|
| | Ratio | 95% CI | 95% CI | P-value |
| DDRD-positive | 0.110 | 0.014 | 0.885 | 0.0380 |
| N Stage 1 vs 0 | 3.276 | 0.938 | 11.450 | 0.0630 |
| N Stage 2&3 vs 0 | 6.991 | 2.212 | 22.087 | 0.0009 |
| Surgical T Stage 2 vs 0&1 | 0.412 | 0.035 | 4.802 | 0.4787 |
| Surgical T Stage 3+ vs 0&1 | 1.147 | 0.141 | 9.309 | 0.8981 |

Using Cox proportional hazards regression, there is a statistically significant effect of the DDRD assay on overall survival. The effect is statistically significant when other important risk factors are included in the model (hazard ratio 0.11, 95% CI (0.014, 0.885), p=0.038). Covariates included in the model were directed by clinical expertise.

### Conclusion

Evidence is provided demonstrating that the DDRD assay, is a significant prognosticator of recurrence free survival (p=0.019) and overall survival (p=0.038) in oesophageal cancer after adjusting for other important covariates.

### Example 2

### Background

Oesophageal cancer is the eighth most common cancer worldwide with 480,000 cases diagnosed annually. In the UK the incidence of the disease in men has risen 68% in the last 45 years with the most common pathological subtype being adenocarcinoma. The reason for this increase in oesophageal adenocarcinoma (OAC) is unclear but development of this tumour is strongly associated with the pre-malignant metaplastic condition, Barrett's oesophagus. This is caused by gastro-oesophageal reflux disease and, along with obesity, smoking and alcohol intake, is one of the strongest risk factors for OAC. Despite efforts to screen for Barrett's oesophagus and pre-operatively select OAC patients who are likely to benefit from potentially curative surgery, survival remains poor. The five year survival rate is 13% and even in early stage loco-regional confined disease this figure rarely exceeds 40%. A significant improvement in overall survival has been demonstrated with neo-adjuvant or peri-operative therapy but the optimal approach for individual patients remains unclear. There is a pressing need to identify biomarkers capable of predicting response, enabling clinicians to stratify patients to the neo-adjuvant therapy which would be most beneficial.

### Oesophageal Adenocarcinoma—Patient Cohort

Considering one of the most active drugs in OAC is the DNA-damaging agent Cisplatin we decided to investigate the efficacy of the DDRD signature in OAC. We analyzed 63 formalin fixed paraffin embedded (FFPE) pre-treatment endoscopic biopsy specimens from early stage OAC patients treated with cisplatin-based neo-adjuvant chemotherapy followed by surgical resection between 2004 and 2010 at the Northern Ireland Cancer Centre (Tables 7 and 8).

The 63 samples used in this example include the 45 samples from Example 1. We profiled an additional small sample set and merged the two sample sets together for this analysis. Accordingly, the methods described in Example 1 are applicable to this example.

Biopsies were reviewed for pathological subtype prior to marking for macrodissection and samples containing at least 70% adenocarcinoma tissue were to be taken forward. The matched resection specimens were scored according to the Mandard Score (<3=pathological response). A sufficient quantity of RNA was extracted from 62 out of the 63 FFPE biopsy specimens (98.4%) and hybridized to the Xcel™ array, a cDNA microarray-based technology optimized for archival FFPE tissue (Almac/Affymetrix). All samples were scored for the DDRD assay.

TABLE 7
Patient Characteristics.

| Patient Characteristics | n = 62 |
|---|---|
| Sex, n (%) | |
| Male | 49 (79) |
| Female | 13 (21) |
| Age, years (%) | |
| <60 | 21 (33.9) |
| 60-69 | 29 (46.8) |
| ≧70 | 12 (19.4) |
| Median | 62 |
| Range | 39-78 |
| ECOG Performance Status, n (%) | |
| 0 | 6 (9.7) |
| 1 | 52 (83.9) |
| 2 | 4 (6.5) |
| Primary Site, n (%) | |
| Oesophagus | 5 (8.1) |
| GOJ, Siewert 1 | 43 (69.4) |
| GOJ, Siewert 2 | 10 (16.1) |
| GOJ, Siewert 3 | 4 (6.5) |
| Chemotherapy treatment, n (%) | |
| ECF/X | 59 (95.2) |
| ECarboX | 2 (3.2) |
| CFU | 1 (1.6) |
| PET Response, n (%) | |
| Responder | 15 (24.2) |
| Non-responder | 33 (53.2) |
| Unknown | 14 (22.6) |

TABLE 8
Tumour Characteristics.

| Tumour Characteristics | n = 61* |
|---|---|
| Tumour stage, n (%) | |
| T1 | 9 (14.8) |
| T2 | 11 (18) |
| T3 | 39 (63.9) |
| T4 | 2 (3.3) |
| Nodal Status, n (%) | |
| N0 | 20 (32.8) |
| N1 | 16 (26.2) |
| N2 | 13 (21.3) |
| N3 | 12 (19.7) |
| Differentiation, n (%) | |
| Well | 4 (6.6) |
| Moderate | 24 (39.3) |
| Moderate/poor | 6 (9.8) |
| Poor | 27 (44.3) |
| Lymphovascular invasion, n (%) | |
| Present | 37 (60.7) |
| Absent | 22 (36.1) |
| Unknown | 2 (3.3) |
| Distal Margin involved, n (%) | |
| Yes | 1 (1.6) |
| No | 60 (98.4) |
| Circumferential Margin involved, n (%) | |
| Yes | 33(54.1) |
| No | 28 (45.9) |

*One pathology report unable to be retrieved

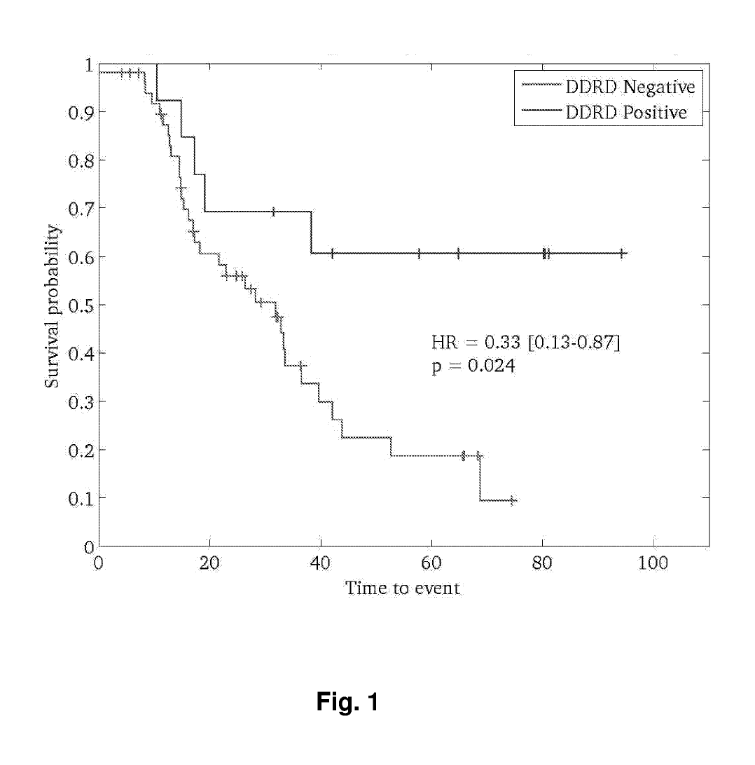

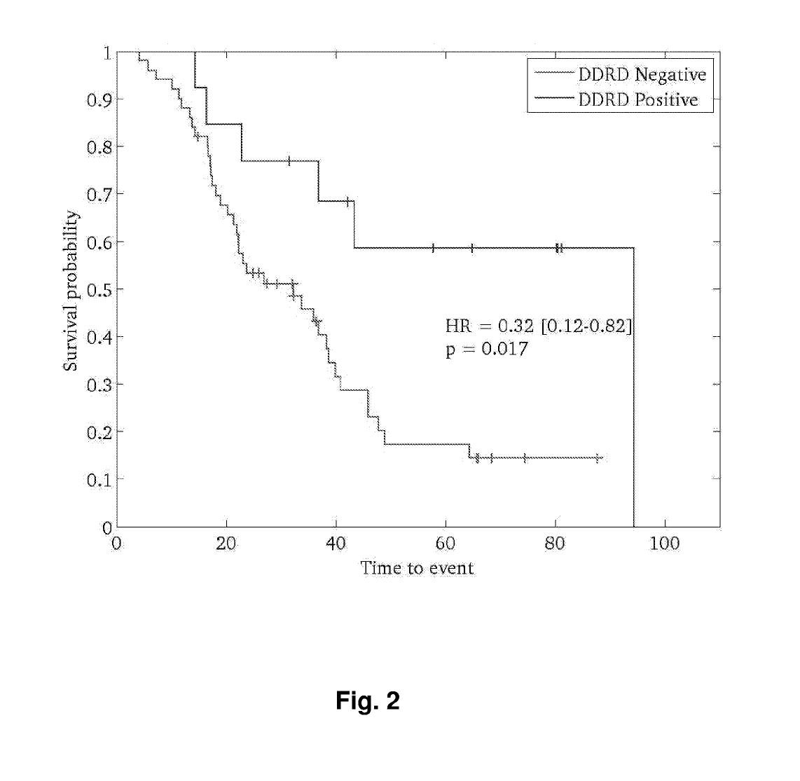

The association between the DDRD score and prognosis was assessed by Kaplan-Meier analysis and Cox Proportional Hazards regression. A total of 13 samples (21%) were characterised as DDRD positive with the remaining 49 samples (79%) DDRD negative. DDRD assay positivity demonstrated a statistically significant association with relapse-free survival (RFS) (HR 0.33; 95% CI 0.13-0.87; p=0.024) resulting in a median RFS of 65.2 months for DDRD+ve patients vs 33.9 months for DDRD-ve patients (FIG. 1). Following multivariable analysis the effect of the DDRD assay on RFS remained statistically significant when other important risk factors were included in the model (HR 0.31, 95% CI (0.110, 0.877), p=0.027) (Table 9). Median overall survival (OS) was significantly higher in DDRD+ve patients (94.3 months vs 32.2 months; HR 0.32; 95% CI 0.12-0.82; p=0.017) (FIG. 2). Following multivariable analysis the effect of the DDRD assay on OS remained statistically significant when other important risk factors were included in the model (HR 0.36, 95% CI (0.132, 0.953), p=0.040). These results indicate that the DDRD assay is a strong prognostic marker in the setting of neo-adjuvant chemotherapy for early stage esophageal adenocarcinoma.

TABLE 9
Multivariable analysis of the predictive value of the DDRD
assay for relapse-free survival

| | Hazard | Lower | Upper | |
|---|---|---|---|---|
| | Ratio | 95% CI | 95% CI | P-value |
| DDRD-positive | 0.31 | 0.11 | 0.877 | 0.027 |
| Surgical T stage 2 vs 0/1 | 0.343 | 0.078 | 1.519 | 0.159 |
| Surgical T stage 3+ vs 0/1 | 0.47 | 0.126 | 1.751 | 0.261 |
| N stage 1 vs 0 | 4.793 | 1.59 | 14.447 | 0.005 |
| N stage 2&3 vs 0 | 7.822 | 2.777 | 22.034 | <0.001 |

TABLE 10
Multivariable analysis of the predictive value of the DDRD
assay for overall survival

| | Hazard | Lower | Upper | |
|---|---|---|---|---|
| | Ratio | 95% CI | 95% CI | P-value |
| DDRD-positive | 0.355 | 0.132 | 0.953 | 0.040 |
| Surgical T stage 2 vs 0/1 | 0.402 | 0.098 | 1.644 | 0.204 |
| Surgical T stage 3+ vs 0/1 | 0.411 | 0.112 | 1.514 | 0.181 |
| N stage 1 vs 0 | 6.679 | 2.090 | 21.341 | 0.001 |
| N stage 2&3 vs 0 | 8.809 | 3.031 | 25.605 | <0.001 |

### DDRD in OAC—Chemotherapy Naive

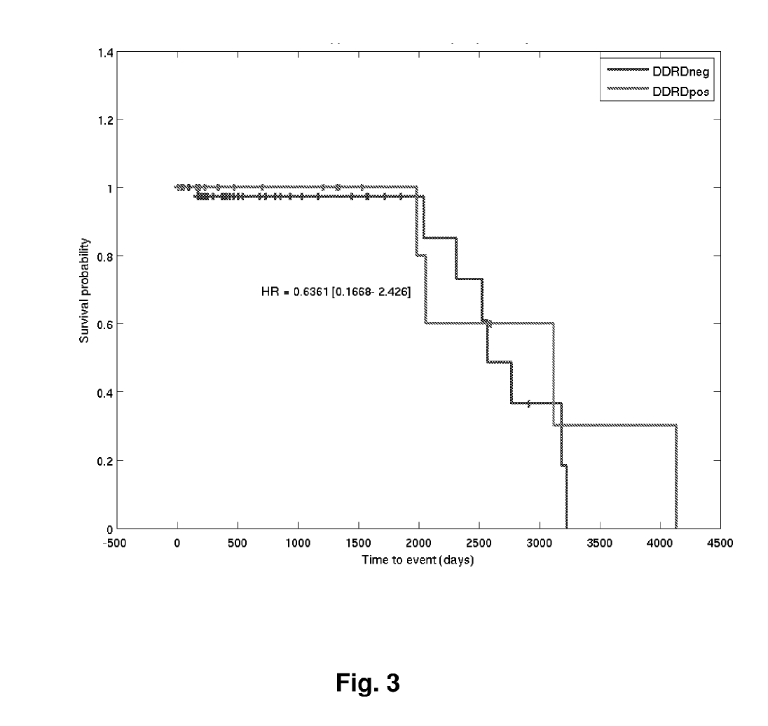

To determine whether the DDRD assay was prognostic independent of treatment it was applied to a dataset of 75 fresh frozen tissue samples derived from potentially curative resections for oesophageal and gastro-oesophageal junction tumours. These resections were carried out between 1992 and 200 and none of the patients received neo-adjuvant chemotherapy. All samples were analysed using a custom-made Agilent 44K 60-mer oligo-microarray. As shown in FIG. 3, no difference in survival was noted between the DDRD positive and negative populations HR 0.64 (95% CI 0.17-2.43). Further Affymetrix-based validation sets are being sought in order to provide a consistent analysis platform between datasets.

### CONCLUSIONS

The DDRD assay could personalise the treatment approach and improve outcomes for early stage EAC patients.
First array-based biomarker to be developed from FFPE tissue in EAC.
Transcriptional profiling successful for 98% of pre-treatment FFPE endoscopic biopsies.
The DDRD assay demonstrates a strong association with prognosis.
A positive DDRD assay result predicted increased RFS (HR 0.31, 95% CI (0.11-0.88), p=0.027) and OS (HR 0.36, 95% CI (0.13-0.95), p=0.04) compared with the assay negative population.
DDRD positive patients have a five-year survival of 59% compared to 17% for DDRD negative patients.
Expansion of this validation set is ongoing to increase the statistical power of the analysis.
The role of the DDRD assay in early stage EAC will be assessed in a prospective clinical trial.

The various embodiments of the present invention are not to be limited in scope by the specific embodiments described herein. Indeed, various modifications of the various embodiments of the invention in addition to those described herein will become apparent to those skilled in the art from the foregoing description and accompanying figures. Such modifications are intended to fall within the scope of the appended claims. Moreover, all embodiments described herein are considered to be broadly applicable and combinable with any and all other consistent embodiments, as appropriate.

Various publications are cited herein, the disclosures of which are incorporated by reference in their entireties.

1. A method of predicting responsiveness of an individual having oesophageal adenocarcinoma (OAC) to treatment with a DNA-damaging therapeutic agent comprising:
a. measuring expression levels of one or more biomarkers in a test cancer sample obtained from the individual, wherein the one or more biomarkers are selected from those listed in Table 2B, 1A, 1B, 2A, 3, and/or 4;
b. deriving a test score that captures the expression levels;
c. providing a threshold score comprising information correlating the test score and responsiveness;
d. and comparing the test score to the threshold score; wherein responsiveness is predicted when the test score exceeds the threshold score and/or wherein a lack of responsiveness is predicted when the test score does not exceed the threshold score.

2. The method of claim 1, wherein the one or more biomarkers are selected from CXCL10, MX1, IDO1, IF144L, CD2, GBP5, PRAME, ITGAL, LRP4, and APOL3 and/or are selected from CDR1, FYB, TSPAN7, RAC2, KLHDC7B, GRB14, AC138128.1, KIF26A, CD274, CD109, ETV7, MFAP5, OLFM4, PI15, FOSB, FAM19A5, NLRC5, PRICKLE1, EGR1, CLDN10, ADAMTS4, SP140L, ANXA1, RSAD2, ESR1, IKZF3, OR2I1P, EGFR, NAT1, LATS2, CYP2B6, PTPRC, PPP1R1A, and AL137218.1.

3. The method of claim 2, comprising measuring the expression level of all of the biomarkers.

4. The method of claim 1 where expression levels are measured using primers and/or probes which bind to at least one of the target sequences set forth in Tables 1A (SEQ ID NO: 1-24), 1B (SEQ ID NO: 25-50), and/or 3 (SEQ ID NO: 51-230).

5. The method of claim 1, wherein the OAC is at early stage, late stage or metastatic disease stage.

6. The method of claim 1, wherein the DNA-damaging therapeutic agent comprises one or more substances selected from: a DNA damaging agent, a DNA repair targeted therapy, an inhibitor of DNA damage signalling, an inhibitor of DNA damage induced cell cycle arrest, a histone deacetylase inhibitor, a heat shock protein inhibitor, and an inhibitor of DNA synthesis.

7. The method of claim 6, wherein the DNA-damaging therapeutic agent comprises one or more of a platinum-containing agent, a nucleoside analogue, an anthracycline, an alkylating agent, an ionising radiation, or a combination of radiation and chemotherapy (chemoradiation).

8. The method of claim 1, wherein the DNA-damaging therapeutic agent comprises a platinum-containing agent.

9. The method of claim 8, wherein the platinum based agent is selected from cisplatin, carboplatin, and oxaliplatin.

10. The method of claim 1, which predicts responsiveness to treatment with the DNA-damaging therapeutic agent together with a further therapy.

11. The method of claim 10 wherein the further therapy is a mitotic inhibitor.

12. The method of claim 11, wherein the mitotic inhibitor is a vinca alkaloid or a taxane.

13. The method of claim 12, wherein the vinca alkaloid is vinorelbine.

14. The method of claim 12, wherein the taxane is paclitaxel or docetaxel.

15. The method of claim 1 which predicts responsiveness to a combination therapy comprising a DNA-damaging therapeutic agent, wherein the combination therapy is selected from:
a. cisplatin/carboplatin and 5-fluorouracil;
b. cisplatin/carboplatin and capecitabine;
c. epirubicin/doxorubicin, cisplatin/carboplatin, and fluorouracil;
d. epirubicin/doxorubicin, oxaliplatin, and capecitabine;
e. cisplatin/carboplatin and paclitaxel;
f. cisplatin/carboplatin and irinotecan; and
g. cisplatin/carboplatin and vinorelbine.

16. The method of claim 15 wherein the combination therapy further comprises one or more of a taxane, a topoisomerase inhibitor, a vinca alkaloid, or chemoradiation.

17. The method of claim 1 wherein the treatment is neoadjuvant treatment and/or adjuvant treatment.

18. The method of claim 1 wherein individuals for whom response is predicted are further treated with the DNA-damaging therapeutic agent.

19. The method of claim 1 wherein individuals for whom lack of response is predicted are not further treated with the DNA-damaging therapeutic agent.

20. The method of claim 1 wherein the treatment is neoadjuvant platinum-based therapy treatment.

21. The method of claim 19 wherein the individuals for whom lack of response is predicted are further treated with a mitotic inhibitor.

22. The method of claim 1 wherein responsiveness comprises or is increased overall survival, progression free survival and/or disease free survival.

23. A method of treating oesophageal adenocarcinoma (OAC) comprising administering a DNA-damaging therapeutic agent to a subject, wherein the subject is predicted to be responsive to the DNA-damaging therapeutic agent on the basis of a test score derived from expression levels of one or more biomarkers in a test cancer sample obtained from the individual, wherein the one or more biomarkers are selected from those listed in Table 2B, 1A, 1B, 2A, 3, and/or 4.

24. A method of treating oesophageal adenocarcinoma (OAC) comprising administering a mitotic inhibitor to a subject, wherein the subject is predicted to be non-responsive to a DNA-damaging therapeutic agent on the basis of a test score derived from expression levels of one or more biomarkers in a test cancer sample obtained from the individual, wherein the one or more biomarkers are selected from those listed in Table 2B, 1A, 1B, 2A, 3, and/or 4.

25. The method of claim 23 wherein the test score has been derived according to the method of claim 1.

26. A kit for predicting responsiveness of an individual having oesophageal adenocarcinoma (OAC) to treatment with a DNA-damaging therapeutic agent comprising primers or probes which hybridize to at least one of the target sequences set forth in Table 3 (SEQ ID NO: 51-230).

27. The kit of claim 26 wherein the primers or probes hybridize to at least 10 of the target sequences set forth in Table 3.

28. The kit of claim 26 further comprising a DNA-damaging therapeutic agent.

29. The kit of claim 28 wherein the DNA-damaging therapeutic agent is provided in a dosage form specifically for treatment of OAC.

30. The kit of claim 29 wherein the treatment is neo-adjuvant or adjuvant treatment.

31. The kit of claim 26 wherein the DNA-damaging therapeutic agent comprises a platinum-based agent.

32. The method of claim 7, wherein the nucleoside analogue is selected from gemcitabine and 5-fluorouracil, or a prodrug thereof.

33. The method of claim 32, wherein the prodrug is capecitabine.

34. The method of claim 7, wherein the anthracycline is selected from epirubicin and doxorubicin.

35. The method of claim 7, wherein the alkylating agent is cyclophosphamide.

36. The method of claim 16, wherein the taxane is paclitaxel.

37. The method of claim 16, wherein the topoisomerase inhibitor is iriniotecan.

38. The method of claim 16, wherein the vinca alkaloid is vinorelbine.

39. The method of claim 24 wherein the test score has been derived according to the method of claim 1.


SEQUENCE LISTING (230 sequences)

SEQ ID NO: 1 - DNA, 541 bp, Homo sapiens
gggaccaaggtggagatcaaacgtaagtgcactttcctaatgctttttcttataaggttttaaatttggagcctttttgtgtttgagatattagctcaggtcaattccaaagagtaccagattctttcaaaaagtcagatgagtaagggatagaaaagtagttcatcttaaggaacagccaagcgctagccagttaagtgaggcatctcaattgcaagattttctctgcatcggtcaggttagtgatattaacagcgaaaagagatttttgtttaggggaaagtaattaagttaacactgtggatcaccttcggccaagggacacgactggagattaaacgtaagtaatttttcactattgtcttctgaaatttgggtctgatggccagtattgacttttagaggcttaaataggagtttggtaaagattggtaaatgagggcatttaagatttgccatgggttgcaaaagttaaactcagcttcaaaaatggatttggagaaaaaaagattaaattgctctaaactgaatgacacaaagt

SEQ ID NO: 2 - DNA, 600 bp, Homo sapiens
tttattggtcttcagatgtggctgcaaacacttgagactgaactaagcttaaaacacggtacttagcaatcgggttgccagcaaagcactggatgcaagccttgccttccagaagcttaccagtcgggttgccagcaaagcagtggatgcaagacttgccctccaggagcttaccatcacaacgaagaagacaaataaatgcataatatatagacgacataaatccatactgtacacatttaagaataaacagtccagtagtaagaggcagtacatattcaatctgctgagaaatgtagacaataactactataagaatcctaatgctacagaagtcactggctgctgggaaaccggggaaaacttggctatggacgtgggggcttgtgtcggactctgaataaagagcagaatgattggcgtcctactgagatacatagtaaagggggcgagggcagggaggaagtggcaagaataacatttgtgaagatgtccaggtgagaaatagaggttttaatgctcaagatgtttccttttcccttttaaatctgacctgtgatttccagcattgctatttcgaatatcactgattgtttttaa

SEQ ID NO: 3 - DNA, 600 bp, Homo sapiens
tgtggcacatatacaccatggaatactatgcagccataaaaaagaatgggatcatgtcctgtgcagcaacgtggatggagctggaagccattatcctaaatgaactcactcagaaacagaaaaccaaataccacatgttctcacttataagtagaagctaaacattgagtacacatggatacaaagaagggaaccgcagacactggggcctacctgaggtcggagcatggaaggagggtgaggatcaaaaaactacctatctggtactatgctttttatctggatgatgaaataatctgtacaacaaaccctggtgacatgcaatttacctatatagcaagcctacacatgtgcccctgaacctaaaaaaaaagttaaaagaaaaacgtttggattattttccctctttcgaacaaagacattggtttgcccaaggactacaaataaaccaacgggaaaaaagaaaggttccagttttgtctgaaaattctgattaagcctctgggccctacagcctggagaacctggagaatcctacacccacagaacccggctttgtccccaaagaataaaaacacctctctaaaaaaaaaaaaaaaa

SEQ ID NO: 4 - DNA, 600 bp, Homo sapiens
tccttatggggcccggtatgtgggctccatggtggctgatgttcatcgcactctggtctacggagggatatttctgtaccccgctaacaagaagagccccaatggaaagctgagactgctgtacgaatgcaaccccatggcctacgtcatggagaaggctgggggaatggccaccactgggaaggaggccgtgttagacgtcattcccacagacattcaccagagggcgccggtgatcttgggatcccccgacgacgtgctcgagttcctgaaggtgtatgagaagcactctgcccagtgagcacctgccctgcctgcatccggagaattgcctctacctggaccttttgtctcacacagcagtaccctgacctgctgtgcaccttacattcctagagagcagaaataaaaagcatgactatttccaccatcaaatgctgtagaatgcttggcactccctaaccaaatgctgtctccataatgccactggtgttaagatatattttgagtggatggaggagaaataaacttattcctccttaaaaaaaaaaaaaaaaaaaaaaaaaaaaaaaaaaaaaaaaaaaaaaaaaaaaaaaaaaa

SEQ ID NO: 5 - DNA, 600 bp, Homo sapiens
cgggcgtggtagcgggcgcctgtagtcccagctactcgggaggctgaggcaggagaatggcgtgaacccgggaggcggagcttgcagtgagccgagatcgcgccactgcactccagcctgggcgacagagcgagactccgtctcaaaaaaaaaaaaaaaaaaaaaaatacaaaaattagccgggcgtggtggcccacgcctgtaatcccagctactcgggaggctaaggcaggaaaattgtttgaacccaggaggtggaggctgcagtgagctgagattgtgccacttcactccagcctgggtgacaaagtgagactccgtcacaacaacaacaacaaaaagcttccccaactaaagcctagaagagcttctgaggcgctgctttgtcaaaaggaagtctctaggttctgagctctggctttgccttggctttgccagggctctgtgaccaggaaggaagtcagcatgcctctagaggcaaggaggggaggaacactgcactcttaagcttccgccgtctcaacccctcacaggagcttactggcaaacatgaaaaatcggcttaccattaaagttctcaatgcaaccataaaaaaaaaa

SEQ ID NO: 6 - DNA, 600 bp, Homo sapiens
tacagatactcagaagccaataacatgacaggagctgggactggtttgaacacagggtgtgcagatggggagggggtactggccttgggcctcctatgatgcagacatggtgaatttaattcaaggaggaggagaatgttttaggcaggtggttatatgtgggaagataattttattcatggatccaaatgtttgttgagtcctttctttgtgctaaggttcttgcggtgaaccagaattataacagtgagctcatctgactgttttaggatgtacagcctagtgttaacattcttggtatctttttgtgccttatctaaaacatttctcgatcactggtttcagatgttcatttattatattcttttcaaagattcagagattggcttttgtcatccactattgtatgttttgtttcattgacctctagtgataccttgatctttcccactttctgttttcggattggagaagatgtaccttttttgtcaactcttacttttatcagatgatcaactcacgtatttggatctttatttgttttctcaaataaatatttaaggttatacatttaaaaaaaaaaaaaaaaaaaaaaaaaaa

SEQ ID NO: 7 - DNA, 600 bp, Homo sapiens
tgagaagtagttactgtgcacatgtgtagatttgcagttctgtggctcctgatggatctgagaagatggacgtggaggatgaaaatctgtctgattattttgaactgatgtttgttgctatggagatgctgcctatatgttgatgttgcagacgttaagtcactagcccacagccttgtattccatactcagagaccctgctacttacttgacatctcaacttgaaagtccaattaatatgcacttcaaactttaataggcttcaaacagaatttctttcattatctctgcaaaacagcttctctcatcatcttgaaattagtgaatggcattttactgttttagttggagtcatttctgtggttttctttcacatcctacataacaatccatcagtaagttctatgagctcttctttgaaaacaaacagaatccaactgtttcattcccacttctgctctggtcaagccactgccaacactcacctttattattgtagcaccctcattgcctagttctgtcccacagatttccaataaaaggtgaataaaatcaggtcactcttctgctaaaaaaaaaaaaaaaaaaaaaaaaaaaaaa

SEQ ID NO: 8 - DNA, 600 bp, Homo sapiens
  misc_feature (1)..(5): n is a, c, g, or t
nnnnntttgctacagccagggttagctcagcaggtgaaaaccccgagggtgggtgaaacccctctggggctcagacatgcaaaccttgggcatctctctgtcccagctggccccgccagccggtaggaagtttcccctgagttctcagttttttcttctgaaaaatgaggggttgtatgcaaggttctcctcctggcctgtggtccccagagaagggcaggaaggaaccttagataattctcatatgcatttaacagacgaggaaactgagacccagagccgtcacatcaatacctcatttgatcttcataagagcacctggaggaggggggtggggtgtttgtgtttgtttaaannnnnnnnngtgaaaaaaatgaagataggcattttgtagacaatctggaagttctggaccggaatccatgatgtagtcagggaagaaatgacccgtgtccagtaaccccaggcctcgagtgtgtggtgtatttttctacataattgtaatcattctatacatacaaattcatgtcttgaccatcatattaatatttggtaagtttctctctctttagagactccacaataaagttttcaacatgg

SEQ ID NO: 9 - DNA, 394 bp, Homo sapiens
  misc_feature (2)..(2): n is a, c, g, or t
tnttntnttttttttttttttttttttttttncatagttgttatcttaaggtgatttccaatttttttttccatttacatttttccacaagcattgtccactttattctgtaaccttttcaactaccattttgaaatttgcttttatccatgtggttgtttgtgatgaactacaggttgctgactttcttccccttctgtnnnnnnnnnnnnnnnnnnnnnnngtnntnnnnctcaagaggatctcatcagtggaatcattagatcaaaggatatgactgttgctcagctctctgtgtgtatgtaaattaataggctgtttatttgagcagttgtaggcttacaaaaatattgagtcaaaagtatagaattcccatatattctcctcttctccc

SEQ ID NO: 10 - DNA, 600 bp, Homo sapiens
atcttcccacctcgatggggggttgctgataagaccttcaggcctccttattaccataggaactgcatgagtgagttcatgggactcatccgaggtcactatgaggcaaagcaaggtgggttcctgccagggggagggagtctacacagcacaatgaccccccatggacctgatgctgactgctttgagaaggccagcaaggtcaagctggcacctgagaggattgccgatggcaccatggcatttatgtttgaatcatctttaagtctggcggtcacaaagtggggactcaaggcctccaggtgtttggatgagaactaccacaagtgctgggagccactcaagagccacttcactcccaactccaggaacccagcagaacctaattgagactggaacattgctaccataattaagagtagatttgtgaagattcttcttcagaatctcatgctttctggtagtattggaggagggggttggttaaaatgaaaattcacttttcatagtcaagtaactcagaacttttatggaaacgcatttgcaaagttctatggctgtcaccttaattactcaataaacttgctggtgttctgtgga

SEQ ID NO: 11 - DNA, 600 bp, Homo sapiens
gggagctaagtatccagcctctcccaaacctctttgaacaaagcttctgtccctcccacacctctcacctcacaggcacatcaggctgcagaatgcgctttagaaagcattgttttagtccaggcacagtggctcacgcctgtaatcccagcactttgggaggccgaggtgggtggatcacaaggttgggagattgagaccatcctggctaacacagtgaaaccctgtctctactaaaaaaatacaaaaaattagcttggcgtggtggtgggcgcctgtagtcccagcagcttgggaggctgaggctggagaatggtgtgaacccaggaggcggagcttgcagtgagccaagatcgcgccactgcactccagcccgggtgacagagcaagactccgtctcaaaaaaaagaaaagaaaaaagaaagcattgttttaattgagaggggcagggctggagaaggagcaagttgtggggagccaggcttccctcacgcagcctgtggtggatgtgggaaggagatcaacttctcctcactctgggacagacgatgtatggaaactaaaaagaacatgcggcaccttaaaaaaaaaaaaaaaaaa

SEQ ID NO: 12 - DNA, 600 bp, Homo sapiens; identical to SEQ ID NO: 2

SEQ ID NO: 13 - DNA, 600 bp, Homo sapiens
atcccaaaggccctttttagggccgaccacttgctcatctgaggagttggacacttgactgcgtaaagtgcaacagtaacgatgttggaaggcttatgattttactgtgtatgtatttgggagaagaaattctgtcagctcccaaaggataaaccagcagttgctttattggtcttcagatgtggctgcaaacacttgagactgaactaagcttaaaacacggtacttagcaatcgggttgccagcaaagcactggatgcaagccttgccttccagaagcttaccagtcgggttgccagcaaagcagtggatgcaagacttgccctccaggagcttaccatcacaacgaagaagacaaataaatgcataatatatagacgacataaatccatactgtacacatttaagaataaacagtccagtagtaagaggcagtacatattcaatctgctgagaaatgtagacaataactactataagaatcctaatgctacagaagtcactggctgctgggaaaccggggaaaacttggctatggacgtgggggcttgtgtcggactctgaataaagagcagaatgattggcaaaaaaaaaaaaaaa

SEQ ID NO: 14 - DNA, 600 bp, Homo sapiens
cgtcttctaaatttccccatcttctaaacccaatccaaatggcgtctggaagtccaatgtggcaaggaaaaacaggtcttcatcgaatctactaattccacaccttttattgacacagaaaatgttgagaatcccaaatttgattgatttgaagaacatgtgagaggtttgactagatgatggatgccaatattaaatctgctggagtttcatgtacaagatgaaggagaggcaacatccaaaatagttaagacatgatttccttgaatgtggcttgagaaatatggacacttaatactaccttgaaaataagaatagaaataaaggatgggattgtggaatggagattcagttttcatttggttcattaattctataagccataaaacaggtaatataaaaagcttccatgattctatttatatgtacatgagaaggaacttccaggtgttactgtaattcctcaacgtattgtttcgacagcactaatttaatgccgatatactctagatgaagttttacattgttgagctattgctgttctcttgggaactgaactcactttcctcctgaggctttggatttgacattgcatttgac

SEQ ID NO: 15 - DNA, 600 bp, Homo sapiens
actcaaatgctcagaccagctcttccgaaaaccaggccttatctccaagaccagagatagtggggagacttcttggcttggtgaggaaaagcggacatcagctggtcaaacaaactctctgaacccctccctccatcgttttcttcactgtcctccaagccagcgggaatggcagctgccacgccgccctaaaagcacactcatcccctcacttgccgcgtcgccctcccaggctctcaacaggggagagtgtggtgtttcctgcaggccaggccagctgcctccgcgtgatcaaagccacactctgggctccagagtggggatgacatgcactcagctcttggctccactgggatgggaggagaggacaagggaaatgtcaggggcggggagggtgacagtggccgcccaaggcccacgagcttgttctttgttctttgtcacagggactgaaaacctctcctcatgttctgctttcgattcgttaagagagcaacattttacccacacacagataaagttttcccttgaggaaacaacagctttaaaagaaaaagaaaaaaaaagtctttggtaaatggcaaaaaaaaaaaaaaaaaa

SEQ ID NO: 16 - DNA, 600 bp, Homo sapiens
gggatttgttaaaatggaggtctttggtgaccttaacagaaagggtttttgaggagtagtggagtggggaggggcagcaggaaggggagattgtacacaccccaggagacaagtcttctagcagttctgccagaatgggcaggagagaagtgccatagagctggaaggctacattgaatagagaaatttctttaacttgttttttaagaagggtgataaaaaggcatgttctgatggtgatagggatgtttccataactggaaagaaattgatgtgcaagagaaagaatataattgcaggaggacttgaagaagttggagagaaaaagcctttagggaccctgaaccaatgaatctgaaattccccaactgccagatgtatcttcatttttcattttccgggagatgtaatatgtcctaaaaatcacagtcgctagattgaaatcaaccttaaaaatcatctagtccaatgtctactcccagtccactacttgaatcccctgtgtcccctcccagtagtcgtcttgacaacctccactgaaaggcaatttctacactccatccaccccaccaccaacccatggttcatgatctcttcgga

SEQ ID NO: 17 - DNA, 600 bp, Homo sapiens
ttactatatcaacaactgataggagaaacaataaactcattttcaaagtgaatttgttagaaatggatgataaaatattggttgacttccggctttctaagggtgatggattggagttcaagagacacttcctgaagattaaagggaagctgattgatattgtgagcagccagaaggtttggcttcctgccacatgatcggaccatcggctctggggaatcctgatggagtttcactcttgtctcccaggctggagtacaatggcatgatctcagcttactgcaacctccgtctcctgggttcaagcgattctcctgcctcagccttccaagtagctgggattacaggtgcccaccaccacacctggctaggttttgtatttttagtagagatggggtttttttcatgttggccaggctgatctggaactcctgacctcaagtgatccacctgccttggcctcccaaagtgctgggattttaggtgtgagccacctcgcctggcaagggattctgttcttagtccttgaaaaaataaagttctgaatcttcaaaaaaaaaaaaaaaaaaaaaaaaaaaaaaaaaaaaaaaaaaaaaaaaa

SEQ ID NO: 18 - DNA, 600 bp, Homo sapiens
gtgtatcatgagccaaccctcaaaggacccgtattacagtgccacgttggaaaacgctacaggaagcatgacctatccacatctttccaagatagacactaacatgtcatgtcccaaacattagcacgtgggggttgagctctgtgcagtaatcgagattgggagaatttgggcagcgcgtgagaagtgctaagctacttgttttctcacttgagcccgggtaggctgtgttggccctcacttgggattctcagcagttacatgaaagttgtgctgataatctcttctcttgtaccaattttagtcaggcagaaaatggtaaacatgagggtgctcttgtgacttaatttttgttcaagggactaaattgcttatgtttattccctgtcagcggagtggagaatgtcattcatcaataaaccaaagccaatagctggagaattgagatctggttgaaagtggtttatggtttacatgctgtactatcctgaggaattgcgagatattgctgaggggaaaaaaaaatgaccttttcttgaaatgtaacttgaaaacaaaataaaatgtggaacataaaaaaaaaaaaaaaaaaaaaaaaaa

SEQ ID NO: 19 - DNA, 600 bp, Homo sapiens
ccagaggcagaaggattgggactaggccaacatagagattggcgatggttgtgagattctaagagtgtgtgtgcatcttgacaatattagaggaggctgagcccaagcaggcacattctcttcgacccctccctcattcagtctgctttggagtctactgaacatcaagcttgctatgagcaggatcttagagctgaggaattggcctcccaatccgaacaggtgttataatcctttcttaataggttgtgctgtggacccaatgtgagggctgtgctggtgtaaatggtgacatattgagctggggggatgctttcggggtggggggactggttccattccatcaaaggccctcttgagagtctatccagggacccattgttttactttaacagaccagaaaagatgtttgttttccatgtcattacccccaggggataccgaatgtgtgggtagaaatttctctgtagattaaaaatcagatttttacatggattcaacaaaggagcgtcacttggatttttgttttcatccatgaatgtagctgcttctgtgtaaaatgccattttgctattaaaaatcaattcacgctggaaaaaa

SEQ ID NO: 20 - DNA, 600 bp, Homo sapiens
gcacgtctacggggctggacagagtgtggttaaccggggaactgggcaagccggcgccgagcctgcgtcagccgtgcaagccgctccttcaggaacttccgcttgtcgctggtgtcgctccgctccttcaggagccagctgtaggtgtccttgtcctgcaggagctgcagcatggccttctgaagctgctggccgtacgtctggagcatgaagaactggatgatcaaagggatgtggctggagatgcgcttgctggcctcctggtgataggccatcaggtgctgaaagatctcctccatggaagagtctgttgccgagctggactggaaagccccaaaatcccaggatttcttcttcttttcttcttccagctccttctctctgaccttctgcaatgcacccctgtatacctggtcctggcagtagacaatctgttccatctggaagtggaggcggatcagcttctcaccttctctctcttgttctgctctaatgtcttcaattttggacttggcggttctgtggaggttaaaaaactcttcaaaattttttatcgccaacttttttgtacaaagttggccttataaagaaagcattgct

SEQ ID NO: 21 - DNA, 600 bp, Homo sapiens
  misc_feature (1)..(107): n is a, c, g, or t
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccaaatgagtgatgcattgaccgttcgtaattcttggatgcaaaagtagaactcaagctacttaataacaatcatggtggcatgggcaccagcaagtcagggtggacaacagccatagttctggagcatggtcctcaagactaccttttgtatgcagagtattaacactttaactcttagatccttggaacataaggaagagaggctggaacaaaaaggggttggcatttggaggtggagaggtagtgtaaggcacaactgtttatcaactggtatctaagtatttcaggccagacacgtggctcacacctctaatcccagcactttgggagctgagccaggaggattgcttgagtctaggagttcaagaccggtctgggcaacatggtgaaaccctgtctctacaaaaaaatacaaaaattagccaggtgtggtggggcacgcctatggtcccagctactggggaggctgagatgggaggatccacctgagc

SEQ ID NO: 22 - DNA, 477 bp, Homo sapiens
ttttttttaattaacttgactttattgatagttacagcacaatttattaattaacttgactttattgatagttacagcacaatctgtccaaaaccaccagaatatacattcttttcaagagctcaaatggaacatttaccacaaaagaccatattctgggcttcaaaataagcctaaataaatacaaaagcatttaggacctatgaatcagaagactgaatatgcacatatacaaaatgagaatcattctctcacatacaaaacttatataggtagtaaagatacagttgattaggtagatttgaatgttgaatcactgacatttcctgaaggtagagctacaaattacttttttaaaaccactaacccacccccaccttacctcacttactctttttggccttaccacctactttagtcataccctatacatgttactcagaccaaatggctctcataaacaatctcagtatatgt

SEQ ID NO: 23 - DNA, 600 bp, Homo sapiens
  misc_feature (229)..(316): n is a, c, g, or t
ttaagaaggtatggaaagagtctgggagtgactaaactatccaatgtcattgaaataaagcaatgaagaataagagtaatttttgttgctttattaaattttttctcacagaattctttataaaaacaccatgtccctaaaatgtcattcaacatatatgcacaccttcgatgtataggacactgatcaaaaaagacagagaaatgtgtccctggtgttttgtttttgnnnnnnnnnnnnnnnnnnnnnnnnnnnnnnnnnnnnnnnnnnnnnnnnnnnnnnnnnnnnnnnnnnnnnnnnnnnnnnnnnnnnnnnngggactacaggcacataccaccacacctggcttcatgttcccggtattagtacaatgccaaaatatttaaaattcttaaaggttaactcaaatatcttaagttttacttcacttacaatttcaataatgctgaaattttgattgaatattgtgtttgtagtgctacctctttttcgttcataagaacaaaagcctatcattctcttagtttctaaaaaatatatgttcatatggtttagatacatatataaatatntacacaaaacaatgttttttgagttgta

SEQ ID NO: 24 - DNA, 429 bp, Homo sapiens
gcccgtgccgccccagccgctgccgcctgcaccggacccggagccgccatgcccaagtgtcccaagtgcaacaaggaggtgtacttcgccgagagggtgacctctctgggcaaggactggcatcggccctgcctgaagtgcgagaaatgtgggaagacgctgacctctgggggccacgctgagcacgaaggcaaaccctactgcaaccacccctgctacgcagccatgtttgggcctaaaggctttgggcggggcggagccgagagccacactttcaagtaaaccaggtggtggagaccccatccttggctgcttgcagggccactgtccaggcaaatgccaggccttgtccccagatgcccagggctcccttgttgcccctaatgctctcagtaaacctgaacacttggaaaaaaaaaaaaaaaaaaa

SEQ ID NO: 25 - DNA, 600 bp, Homo sapiens
caaccaggaagaaccgtaccagaaccactccggccgattcgtctgcactgtacccggctactactacttcaccttccaggtgctgtcccagtgggaaatctgcctgtccatcgtctcctcctcaaggggccaggtccgacgctccctgggcttctgtgacaccaccaacaaggggctcttccaggtggtgtcagggggcatggtgcttcagctgcagcagggtgaccaggtctgggttgaaaaagaccccaaaaagggtcacatttaccagggctctgaggccgacagcgtcttcagcggcttcctcatcttcccatctgcctgagccagggaaggaccccctcccccacccacctctctggcttccatgctccgcctgtaaaatgggggcgctattgcttcagctgctgaagggagggggctggctctgagagccccaggactggctgccccgtgacacatgctctaagaagctcgtttcttagacctcttcctggaataaacatctgtgtctgtgtctgctgaacatgagcttcagttgctactcggagcattgagagggaggcctaagaataataacaatccagtgcttaagagtca

SEQ ID NO: 26 - DNA, 600 bp, Homo sapiens
gggtcgacccttgccactacacttcttaaggcgagcatcaaaagccggggaggttgatgttgaacagcacactttagccaagtatttgatggagctgactctcatcgactatgatatggtgcattatcatccttctaaggtagcagcagctgcttcctgcttgtctcagaaggttctaggacaaggaaaatggaacttaaagcagcagtattacacaggatacacagagaatgaagtattggaagtcatgcagcacatggccaagaatgtggtgaaagtaaatgaaaacttaactaaattcatcgccatcaagaataagtatgcaagcagcaaactcctgaagatcagcatgatccctcagctgaactcaaaagccgtcaaagaccttgcctccccactgataggaaggtcctaggctgccgtgggccctggggatgtgtgcttcattgtgccctttttcttattggtttagaactcttgattttgtacatagtcctctggtctatctcatgaaacctcttctcagaccagttttctaaacatatattgaggaaaaataaagcgattggtttttcttaaggtaaaaaaaaaaaaaaaaaa

SEQ ID NO: 27 - DNA, 600 bp, Homo sapiens
cagaaaggcccgcccctccccagacctcgagttcagccaaaacctccccatggggcagcagaaaactcattgtccccttcctctaattaaaaaagatagaaactgtctttttcaataaaaagcactgtggatttctgccctcctgatgtgcatatccgtacttccatgaggtgttttctgtgtgcagaacattgtcacctcctgaggctgtgggccacagccacctctgcatcttcgaactcagccatgtggtcaacatctggagtttttggtctcctcagagagctccatcacaccagtaaggagaagcaatataagtgtgattgcaagaatggtagaggaccgagcacagaaatcttagagatttcttgtcccctctcaggtcatgtgtagatgcgataaatcaagtgattggtgtgcctgggtctcactacaagcagcctatctgcttaagagactctggagtttcttatgtgccctggtggacacttgcccaccatcctgtgagtaaaagtgaaataaaagctttgactagaaaaaaaaaaaaaaaaaaaaaaaaaaaaaaaaaaaaaaaaaaaaaaaaaaaaaaaaaaaaaaaaa

SEQ ID NO: 28 - DNA, 600 bp, Homo sapiens
  misc_feature (302)..(302): n is a, c, g, or t
tcagcactgagtgttcaaagacagtaggacgtcggttgctgacctgcctcttagaagctagtttaactcagcgggtaaggatctaggacttctacattagttaccactgtaatgataacaccaccagaaaagtctgtagtttaatatttcccaccttatgcctgtttcttcattcacgcaaagaaaataaaaatataatacctaagcctctttgtattacataaagcaaaatgcaaagcactgtatcttccaaatacttcctcttgatatggtggaattatagagtagtatcatttgtaacntgaaatgtcttctagggttgctatgcgaaagcaagactgtggtttcattccaatttcctgtatatcggaatcatcaccatctgtgtatgtgtgattgaggtgttggggatgtcctttgcactgaccctgaactgccagattgacaaaaccagccagaccatagggctatgatctgcagtagtcctgtggtgaagagacttgtttcatctccgggaaatgcaaaaccatttataggcatgaagccctacatgatcacttgcagggtgancctcctcccatccttttcccttttagggtc

SEQ ID NO: 29 - DNA, 600 bp, Homo sapiens
gcctgggacgctgctgctgttcaggaaacgatggcagaacgagaagctcgggttggatgccggggatgaatatgaagatgaaaacctttatgaaggcctgaacctggacgactgctccatgtatgaggacatctcccggggcctccagggcacctaccaggatgtgggcagcctcaacataggagatgtccagctggagaagccgtgacacccctactcctgccaggctgcccccgcctgctgtgcacccagctccagtgtctcagctcacttccctgggacattctcctttcagcccttctgggggcttccttagtcatattcccccagtggggggtgggagggtaacctcactcttctccaggccaggcctccttggactcccctgggggtgtcccactcttcttccctctaaactgccccacctcctaacctaatccccccgccccgctgcctttcccaggctcccctcaccccagcgggtaatgagcccttaatcgctgcctctaggggagctgattgtagcagcctcgttagtgtcaccccctcctccctgatctgtcagggccacttagtgataataaattcttcccaactgca

SEQ ID NO: 30 - DNA, 600 bp, Homo sapiens
ggattcagccagtgcggattttccatataatccaggacaaggccaagctataagaaatggagtcaacagaaactcggctatcattggaggcgtcattgctgtggtgattttcaccatcctgtgcaccctggtcttcctgatccggtacatgttccgccacaagggcacctaccataccaacgaagcaaagggggcggagtcggcagagagcgcggacgccgccatcatgaacaacgaccccaacttcacagagaccattgatgaaagcaaaaaggaatggctcatttgaggggtggctacttggctatgggatagggaggagggaattactagggaggagagaaagggacaaaagcaccctgcttcatactcttgagcacatccttaaaatatcagcacaagttgggggaggcaggcaatggaatataatggaatattcttgagactgatcacaaaaaaaaaaaacctttttaatatttctttatagctgagttttcccttctgtatcaaaacaaaataatacaaaaaatgcttttagagtttaagcaatggttgaaatttgtaggtaatatctgtcttattttgtgtgtgtttagaggt

SEQ ID NO: 31 - DNA, 600 bp, Homo sapiens
atgtccaaaaagatacagaagaactaaagagctgtggtatacaagacatatttgttttctgcaccagaggggaactgtcaaaatatagagtcccaaaccttctggatctctaccagcaatgtggaattatcacccatcatcatccaatcgcagatggagggactcctgacatagccagctgctgtgaaataatggaagagcttacaacctgccttaaaaattaccgaaaaaccttaatacactgctatggaggacttgggagatcttgtcttgtagctgcttgtctcctactatacctgtctgacacaatatcaccagagcaagccatagacagcctgcgagacctaagaggatccggggcaatacagaccatcaagcaatacaattatcttcatgagtttcgggacaaattagctgcacatctatcatcaagagattcacaatcaagatctgtatcaagataaaggaattcaaatagcatatatatgaccatgtctgaaatgtcagttctctagcataatttgtattgaaatgaaaccaccagtgttatcaacttgaatgtaaatgtacatgtgcagatattcctaaagttttattgac

SEQ ID NO: 32 - DNA, 600 bp, Homo sapiens
ggtttccttcccaggacagctgcagggtagagatcattttaagtgcttgtggagttgacatccctattgactctttcccagctgatatcagagacttagacccagcactccttggattagctctgcagagtgtcttggttgagagaataacctcatagtaccaacatgacatgtgacttggaaagagactagaggccacacttgataaatcatggggcacagatatgttcccacccaacaaatgtgataagtgattgtgcagccagagccagccttccttcaatcaaggtttccaggcagagcaaataccctagagattctctgtgatataggaaatttggatcaaggaagctaaaagaattacagggatgtttttaatcccactatggactcagtctcctggaaataggtctgtccactcctggtcattggtggatgttaaacccatattcctttcaactgctgcctgctagggaaaactgctcctcattatcatcactattattgctcaccactgtatcccctctacttggcaagtggttgtcaagttctagttgttcaataaatgtgttaataatgcttaaaaaaaaaaaaaaaaaa

SEQ ID NO: 33 - DNA, 600 bp, Homo sapiens
attccaggaagcatgggattttattttgcttgattttgggcacatgaaataatagctctaggaaaatgcgcatcttaatgactctttgtaaagagaggcatttcttacaactgtgatgtttgcttacataaaagttacctcataagttaattctaacttttattcttgaattttatttcatttcaatagcttgtttcatttgcacgcctttgtattttgattgacctgtagaatggatgttaggaaactcaaaattgaacacagtgaaacaaatggtatttgaagaaatgtaatatcttttatattctatttatgatatccataatcaaatgagattattttaccacataaatgttttaaatatcagatttttagtttgcagttttaggaaaatgctttagatagaaaaggttcttatgcattgaatttggagtactaccaacaatgaatgaatttattttttatattcttacacattttattggtcattgtcacagatagtaaatactaaaaatttcaggtcagtttgttttgaaactgaaattggaaataaatctggaaatgttttgttgcactaaaataataaaatgaattgtactg

SEQ ID NO: 34 - DNA, 600 bp, Homo sapiens
taggccagccctgtcaccacctccactgccatgaccaggccgaaggcagggaacgccctccccagtcccgctgtccagcaaggccccgagacttttcttctgtgatttccaaaagcaaggcagccgtgctgttctagttcctctccatccgccacctcccctcccgctgccccagaagtttctatcattccatggagaaagctgtgttccaatgaatcctacctcttgcccagtcccaggcagagtaagcagggcccacctagggaccaagaaagagtaggaagaaggggacgagccgggagcaaaaccacctcagacacccgggccttctcagccttctccccgcggccagctgggtctccggggaccctgggccctgggccgcccattcctggccctcccgctgcatctcagacctgacacccaacggggggatgtggtggcctgtgcccaccttctctccctcctcccgacccgccccctcgcccccacccctgtgtgtttcgccagttaagcacctgtgactccagtacctactactggttttgggttggttgttctgtcttttttttaattaaataaaaacatttttaaaatgtt

SEQ ID NO: 35 - DNA, 600 bp, Homo sapiens
tgctcagaccagctcttccgaaaaccaggccttatctccaagaccagagatagtggggagacttcttggcttggtgaggaaaagcggacatcagctggtcaaacaaactctctgaacccctccctccatcgttttcttcactgtcctccaagccagcgggaatggcagctgccacgccgccctaaaagcacactcatcccctcacttgccgcgtcgccctcccaggctctcaacaggggagagtgtggtgtttcctgcaggccaggccagctgcctccgcgtgatcaaagccacactctgggctccagagtggggatgacatgcactcagctcttggctccactgggatgggaggagaggacaagggaaatgtcaggggcggggagggtgacagtggccgcccaaggcccacgagcttgttctttgttctttgtcacagggactgaaaacctctcctcatgttctgctttcgattcgttaagagagcaacattttacccacacacagataaagttttcccttgaggaaacaacagctttaaaagaaaaagaaaaaaaaagtctttggtaaatggcaaaaaaaaaaaaaaaaaaaaaaaaa

SEQ ID NO: 36 - DNA, 600 bp, Homo sapiens
tccccagacaccgccacatggcttcctcctgcgtgcatgtgcgcacacacacacacacacgcacacacacacacacacactcactgcggagaaccttgtgcctggctcagagccagtctttttggtgagggtaaccccaaacctccaaaactcctgcccctgttctcttccactctccttgctacccagaaatcatctaaatacctgccctgacatgcacacctcccctgccccaccagcccactggccatctccacccggagctgctgtgtcctctggatctgctcgtcattttccttcccttctccatctctctggccctctacccctgatctgacatccccactcacgaatattatgcccagtttctgcctctgagggaaagcccagaaaaggacagaaacgaagtagaaaggggcccagtcctggcctggcttctcctttggaagtgaggcattgcacggggagacgtacgtatcagcggccccttgactctggggactccgggtttgagatggacacactggtgtggattaacctgccagggagacagagctcacaataaaaatggctcagatgccacttcaaagaaaaaaaaaa

SEQ ID NO: 37 - DNA, 420 bp, Homo sapiens
gggcggttctccaagcacccagcatcctgctagacgcgccgcgcaccgacggaggggacatgggcagagcaatggtggccaggctcgggctggggctgctgctgctggcactgctcctacccacgcagatttattccagtgaaacaacaactggaacttcaagtaactcctcccagagtacttccaactctgggttggccccaaatccaactaatgccaccaccaaggtggctggtggtgccctgcagtcaacagccagtctcttcgtggtctcactctctcttctgcatctctactcttaagagactcaggccaagaaacgtcttctaaatttccccatcttctaaacccaatccaaatggcgtctggaagtccaatgtggcaaggaaaaacaggtcttcatcgaatctactaattcca

SEQ ID NO: 38 - DNA, 600 bp, Homo sapiens
  misc_feature (187)..(187): n is a, c, g, or t
accctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgncacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttanntngtanannannatgtccattttcttgtttattttgtgtttaataaaatgttcagtttaacatcccannngagaaagttaaaaaa

SEQ ID NO: 39 - DNA, 523 bp, Homo sapiens
ctcctggttcaaaagcagctaaaccaaaagaagcctccagacagccctgagatcacctaaaaagctgctaccaagacagccacgaagatcctaccaaaatgaagcgcttcctcttcctcctactcaccatcagcctcctggttatggtacagatacaaactggactctcaggacaaaacgacaccagccaaaccagcagcccctcagcatccagcaacataagcggaggcattttccttttcttcgtggccaatgccataatccacctcttctgcttcagttgaggtgacacgtctcagccttagccctgtgccccctgaaacagctgccaccatcactcgcaagagaatcccctccatctttgggaggggttgatgccagacatcaccaggttgtagaagttgacaggcagtgccatgggggcaacagccaaaataggggggtaatgatgtaggggccaagcagtgcccagctgggggtcaataaagttacccttgtacttgcaaaaaaaaaaaaaaaaaaa

SEQ ID NO: 40 - DNA, 600 bp, Homo sapiens
gccatcaagaatttactgaaagcagttagcaaggaaaggtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatcatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtataaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttattt

SEQ ID NO: 41 - DNA, 513 bp, Homo sapiens
gggaaatcagtgaatgaagcctcctatgatggcaaatacagctcctattgataggacatagtggaagtgggctacaacgtagtacgtgtcgtgtagtacgatgtctagtgatgagtttgctaatacaatgccagtcaggccacctacggtgaaaagaaagatgaatcctagggctcagagcactgcagcagatcatttcatattgcttccgtggagtgtggcgagtcagctaaatggcaggggcagcaagatggtgttgcagacccaggtcttcatttctctgttgctctggatctctggtgcctacggggacatcgtgatgacccagtctccagactccctggctgtgtctctgggcgagagggccaccatcaagtgcaagtccagccagagtattttatataggtccaacaacaagaactacttagcttggtaccagcagaaagcaggacagcctcctaaattgttcatttactgggcatctacccgggaatccggggtccctgaccgatt

SEQ ID NO: 42 - DNA, 600 bp, Homo sapiens
aacgaaagtctagcctttcgtacccgtatatataaagacacccctgttctgattggacaaggcagcctttcccctgcagctcgattggtggagacgcccactccctgacagaacatctcctgcatgtagaccaaatattaaaactttcctccgtccatctttaactgctggtgttttcaaccctttcccctctgtgccatgtttctagcttttatttaaaacgtactttggttttccttggcaaaattgtgtctagctactaggatgacgtgtcttaatttttttttaaatgttggcgctgaaactggctttgatcaacgttttaaaaagacgcgcgctagttgtgattggccaagtgatttcttcttaccctcttaagtttagaaaggttaatttcatatcttgatttgtctatttaaacttggagatattttcaataatttgttccaaatgcaccatgactattaactcataagtaacaatatgaaacctgatgttaagctacatgaacacatttaatttcaccacaatatgacatcctcatatgaaagcactctcttatcttttacaagttcaactggtatttgtgtaatctgctgt

SEQ ID NO: 43 - DNA, 600 bp, Homo sapiens
  misc_feature (102)..(104): n is a, c, g, or t
tgctaccatgcctgactagtttttgtatttttagtagagacagggtttgaccatattggccaggttggtcttggactcctgacaagtgatccgccctcctcnnncncncgaagtgctagggttacnaggtgtgaaccaccatgcctaactatcgttgctactttctattggaagagaaggcagccctgatttagtctgtttacagtctgcattatgtggagaatagagagccatcatagtccctaaaactttccttgccagttaacccagcaggacaacctgtctttgtctcttgacaactgttaactgagaacagggcccttgctcctctaggtgtgcacattaaggactttgcacagtgtggatgtagctcatgctgctctgccntnnagtacatgctgcttgaattttcatcatnancctccacnccttncacctncnngnnaaaaaaaaagcgtgcaggaagtagcatttcagatccttctccaccacctctgcttcccttctcccttcttttcctccttgcagcattccctttagtacnagggagggatggtggttgaaaatggggggaatgatgttgctcagaaaaaaaaaaaa

SEQ ID NO: 44 - DNA, 600 bp, Homo sapiens
  misc_feature (166)..(166): n is a, c, g, or t
ataatgctggaaacagaagcaccaaactgattgtgcaattactccttttgtagaagaggccaaaatcctcctcctccttcctttctcctatattcactcctccaggatcataaagcctccctcttgtttatctgtgtctgtctgtctgattggttagatttggctncccttccaagctaatggtgtcaggtggagaacagagcaaccttccctcggaaggagacaattcgaggtgctggtacatttcccttgttttctatgttcttctttctagtgggtctcatgtagagatagagatatttttttgttttagagattccaaagtatatatttttagtgtaagaaatgtaccctctccacactccatgatgtaaatagaaccaggaataaatgtgtcattgtgataatcccatagcaatttatggtaagaacaagacccctttccctcaccaccgagtctcgtggtctgtgtctgtgaaccagggcaggtaattgtgacactgcatctcatagaactctgcctgcccagatttttgtgtgctcacctcaatgggtgaaaaataaagtctgtgtaaactgttaaaaaaaaaaaaaaaaaaa

SEQ ID NO: 45 - DNA, 497 bp, Homo sapiens
  misc_feature (383)..(384): n is a, c, g, or t
tcctcagacccagtaattccacccctaggaatccagcttacacacacaagaaagaaaagataaatgtacaaggttagtcactgcacagtgagacagcaaaagattagaaagaacccaagtgattattgatctgggttttattcctttatagcccaaccatatgatggaatactataatgttgtaaaaatgggttaagagttctttatgaattggtgtggaaacatcgccaagatatgaaagccaaatgcagaaaaatatatgtggtatgctattatctatgtgaaaaagacattactattctctggaaggataaacacaaatttgagaatggtggatatctggggtgagaggtatccttttcactgttctttaaaagttttgnnattttggtgtttgcctattcaaaaaaatggttaaaatcagttgccaccaattaaaaattaggagaatgcatataaagaannnaanttcctgttaaaaaaaaaaaaaaaaaaaa

SEQ ID NO: 46 - DNA, 600 bp, Homo sapiens
gcccatagtcccatctttttacaggcattttttacacctggagcagccagaggacgcatgcatggctcttcggaaggtaatttagggatcacccatgtaagtttcctaaggatttctttaacatggttcttctgattcagtccggccaattaaatctaaatccacccctgaaagccatctggtgtggataacaagcccacaaatgagcagtcagctttttgtgccctttagggcctgggacaaccacgggatctaaaaggggctggaactagaggtcttgagctcctgttcctaaaatcatcttcatcctatatctgcagccttctcctgccacggcatgcacccacacatgcgagcctcccgggtactgtcatcctgaattctgagaccatccagcacttcctttagttttgccctggtgctgttgacttttgtttactgaagagtgtgctggaggcaggacaagggacatggaaggctgcaatttaagagtctaaaaggttttagaatcctgaaggaggtttaacaagctgaattgaagaataatacctttctcaactggagagaatttacatgattgcattattgttaaaattaaca

SEQ ID NO: 47 - DNA, 600 bp, Homo sapiens
atcatttagttgaatcattataagtctaggactgtctgtagatgtaaatttgttaagaattaggactcaagagtagaattcctttaatccacatagacttacaatggtgctgtgcacatggagcccctaaatcattgctgactgagtagatttcccagggtaagcccaagaagttactcctagaaggggctggtaggggaaagagccaacatcccacatgcctgcccactttgggtctggtcccaagaaacaaactccagtggcctcgaaaatttaatattgctgtcagaagggcctccccttcaaaggaacaggtcctgatagctcttgttatatgcaaagtggaaaggtaacgtgactgttctctgcatttcctgcctttcaattgagtgaagacagacagatgatttattgggcatttcctagcctccccttcaccataggaaaccagactgaaaaaaaggtgcaaattttaaaaagatgtgtgagtatcttgagggggctgggggagaattcctgtgtaccactaaagcaaaaaaagaaaactctctaacagcaggacctctgatctggaggcatattgaccataaatttacgcca

SEQ ID NO: 48 - DNA, 491 bp, Homo sapiens
tttttctgagcaacatcattccccccattttcaaccaccatccctccctggtactaaagggaatgctgcaaggaggaaaagaagggagaagggaagcagaggtggtggagaaggatctgaaatgctacttcctgcacgctttttttcttcttggaggtggaaggagtggaggatgatgatgaaaattcaagcagcatgtactagacggcagagcagcatgagctacatccacactgtgcaaagtccttaatgtgcacacctagaggagcaagggccctgttctcagttaacagttgtcaagagacaaagacaggttgtcctgctgggttaactggcaaggaaagttttagggactatgatggctctctattctccacataatgcagactgtaaacagactaaatcagggctgccttctcttccaatagaaagtagcaacgatagttaggcatggtggttcacaccttgtaaccctagcacttcgtgggcag

SEQ ID NO: 49 - DNA, 600 bp, Homo sapiens
atcagaacaatttcatgttatacaaataacatcagaaaaatatcttaaattatatggcatattctattgattcatccacaaatttataagtccttaccacctttcattatattggtactaggcattatagtagtgctaggcactatagtaatgctggggtataaacaagaataaaacaaaataagttccttatttcaggtaacttacagtataggtcagtggttcttagcttgctttttaattatgaattcctttgaaagtctagtaaaataatccaacaccattattccccattgcacatacccccagatgttttagacatattttcaattgctccatggaccttaagaaaacttggttggtgtgcagtttggtgtattatgggtaagactggacctggtgttagaaaatctgcatttgaggctttgttctgacagtgtctagtgtaaacatgggcagaccacttaaacctctctttagtcttctctgtagaatgatgataataccatctaattagcaggattgttgttttattcagtgagacagcatatgtaaataacttagtaaaataaaaagcaacgtgtttataatggtaaaaaa

SEQ ID NO: 50 - DNA, 600 bp, Homo sapiens
tgggaatcatgaactccttcgtcaacgacatcttcgaacgcatcgcgggtgaggcttcccgcctggcgcattacaacaagcgctcgaccatcacctccagggagatccagacggccgtgcgcctgctgctgcccggggagttggccaagcacgccgtgtccgagggcaccaaggccgtcaccaagtacaccagcgctaagtaaacttgccaaggagggactttctctggaatttcctgatatgaccaagaaagcttcttatcaaaagaagcacaattgccttcggttacctcattatctactgcagaaaagaagacgagaatgcaaccatacctagatggacttttccacaagctaaagctggcctcttgatctcattcagattccaaagagaatcatttacaagttaatttctgtctccttggtccattccttctctctaataatcatttactgttcctcaaagaattgtctacattacccatctcctcttttgcctctgagaaagagtatataagcttctgtaccccactggggggttggggtaatattctgtggtcctcagccctgtaccttaataaatttgtatgccttttctctt

SEQ ID NO: 51 - DNA, 180 bp, Homo sapiens
gaattctactatttatgtgatccttttggagtcagacagatgtggttgcatcctaactccatgtctctgagcattagatttctcatttgccaataataatacctcccttagaagtttgttgtgaggattaaataatgtaaataaagaactagcataacactcagcatctagtaagtgctc

SEQ ID NO: 52 - DNA, 117 bp, Homo sapiens
gagggggtagcaagttcaccacagtgttaatgggggtcccaaggtattcttcccccaggcctaggtatagggctattactcctctctgctccaggtgtagacatacatttacatttt

SEQ ID NO: 53 - DNA, 257 bp, Homo sapiens
tatggctgatgtgggcaccgtctgtgacccggctcggagctgtgccattgtggaggatgatgggctccagtcagccttcactgctgctcatgaactgggtaaagtaggggtggatgagaaaggtattagggaggagaaggtgggggagggggtagcaagttcaccacagtgttaatgggggtcccaaggtattcttcccccaggcctaggtatagggctattactcctctctgctccaggtgtagacatacatttac

SEQ ID NO: 54 - DNA, 155 bp, Homo sapiens
acacacgcctccgatacagcttcttcgtgccccggccgaccccttcaacgccacgccccactccccaggactggctgcaccgaagagcacagattctggagatccttcggcggcgcccctgggcgggcaggaaataacctcactatcccggctgc

SEQ ID NO: 55 - DNA, 70 bp, Homo sapiens
ctggatgacacagtgtgactccatctcaaaaaaagaaaaaaaagggacaaagtatattggtccaaaaaag

SEQ ID NO: 56 - DNA, 215 bp, Homo sapiens
gaaccctttttcaatatcactcctgtatcaaacagatacagtgttcctgacccattgttatttgtcaatttgtccaattttgtacagccgctactttatcatatttaatacattttgtgacttgggcatgtggaacataattttgttattttcactgcttttactcccctaaaagatttggaatatgtgtgggaaaagacatgcataacagaaca

SEQ ID NO: 57 - DNA, 298 bp, Homo sapiens
gtggaggaaactaaacattcccttgatggtctcaagctatgatcagaagactttaattatatattttcatcctataagcttaaataggaaagtttcttcaacaggattacagtgtagctacctacatgctgaaaaatatagcctttaaatcatttttatattataactctgtataatagagataagtccattttttaaaaatgttttccccaaaccataaaaccctatacaagttgttctagtaacaatacatgagaaagatgtctatgtagctgaaaataaaatgacgtcacaagac

SEQ ID NO: 58 - DNA, 251 bp, Homo sapiens
aattggtgtgtcattgactcttctattttgattttcttttctgtgtaattcagtggtttaatttgacattaagggatacaagcctgaattctagattataattatttaatgaaatagagttcacattctgaattgaagaaaatacttatagcttttgaaaagggatactacattttatcgtatgtgtacagactattgagattgtgtctctgtataataaatttattgcactagcattataacaatttgat

SEQ ID NO: 59 - DNA, 199 bp, Homo sapiens
gctttctctcaccagggaaggtgtgggaaggacttgtgaaatacatattcgaggaaaaactatgcacaaggccgtgcatttaaaaataaactccctaaggctggggtgaaacctgctacggtctgcgcaagttgactgttaatgaatttgattctcaggtgtgagtgattaaaagaacactgatcatgtcattttcttt

SEQ ID NO: 60 - DNA, 255 bp, Homo sapiens
gtgtgatggatcccctttaggttatttaggggtatatgtcccctgcttgaaccctgaaggccaggtaatgagccatggccattgtccccagctgaggaccaggtgtctctaaaaacccaaacatcctggagagtatgcgagaacctaccaagaaaaacagtctcattactcatatacagcaggcaaagagacagaaaattaactgaaaagcagtttagagactgggggaggccggatctctagagccatcctgct

SEQ ID NO: 61 - DNA, 202 bp, Homo sapiens
caatctctttggtacacaggaagctttataaaatttcattcacgaatctcttattttgggaagctgttttgcatatgagaagaacactgttgaaataaggaactaaagctttatatattgatcaaggtgattctgaaagttttaatttttaatgttgtaatgttatgttattgttaattgtactttattatgtattcaatag

SEQ ID NO: 62 - DNA, 175 bp, Homo sapiens
ttgccttctaaatatactgaaatgatttagatatgtgtcaacaattaatgatcttttattcaatctaagaaatggtttagtttttctctttagctctatggcatttcactcaagtggacaggggaaaaagtaattgccatgggctccaaagaatttgctttatgtttttagctat

SEQ ID NO: 63 - DNA, 277 bp, Homo sapiens
acactaacatttccagtagtcacatgtgattgttttgttttcgtagaagaatactgcttctattttgaaaaaagagttttttttctttctatggggttgcagggatggtgtacaacaggtcctagcatgtatagctgcatagatttcttcacctgatctttgtgtggaagatcagaatgaatgcagttgtgtgtctatattttcccctctcaaaatcttttagaatttttttggaggtgtttgttttctccagaataaaggtattacttctcgtgcc

SEQ ID NO: 64 - DNA, 251 bp, Homo sapiens
agtttcaacccctcagaatccagcaatttccaaacatcctcctccaccacctggtcatggttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaattaaa

SEQ ID NO: 65 - DNA, 274 bp, Homo sapiens
ccatgtggtcaacatttggagtttttggtctcctcagagagctccatcacaccagtaaggagaagcaatataagtgtgattgcaagaatggtagaggaccgagcacagaaatcttagagatttcttgtcccctctcaggtcatgtgtagatgcgataaatcaagtgattggtgtgcctgggtctcactacaagcagcctatctgcttaagagactctggagtttcttatgtgccctggtggacacttgcccaccatcctgtgagtaaaagtgaa

SEQ ID NO: 66 - DNA, 48 bp, Homo sapiens
gctttatttcagtgtgggtgctatttggcaagttggaaaatagcattt

SEQ ID NO: 67 - DNA, 235 bp, Homo sapiens
cgtgccctctggatttggcttctttacctcagatttcttcctcagttctccttctgcctccccatcaggctgcggtccctcagccccacgtcccatcacgtcactacccccagcccaggccccgaaacatcatcctgaacttagtcccattcttcccaccacgactgtcccacactttccaccacagcacagcagaatcgctggcagggcccttaggcatgcctggcttatcctc

SEQ ID NO: 68 - DNA, 254 bp, Homo sapiens
gttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccaggatgtcca

SEQ ID NO: 69 - DNA, 262 bp, Homo sapiens
taatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaa

SEQ ID NO: 70 - DNA, 205 bp, Homo sapiens
actgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaaggg

SEQ ID NO: 71 - DNA, 165 bp, Homo sapiens
ttttggttgtggatccagtcacctctgaacatgaactgacatgtcaggctgagggctaccccaaggccgaagtcatctggacaagcagtgaccatcaagtcctgagtggagattagatcctgaggaaaaccatacagctgaattggtcatcccagaactacctct

SEQ ID NO: 72 - DNA, 228 bp, Homo sapiens
catggattagctggaagatctgtatttgatggaagaccttgaaattattggaagacatggatttcctggaagacgtggattttcctggaagatctggatttggtggaagaccagtaattgctggaagactggatttgctggaagacttgatttactggaagacttggagcttcttggaagacatggattgtccggaagacatggattgtctggaagatgtggattttc

SEQ ID NO: 73 - DNA, 238 bp, Homo sapiens
tatgtctaaaagagctcgctggcaagctgcctcttgagtttgttataaaagcgaactgttcacaaaatgatcccatcaaggccctcccataattaacactcaaaactatttttaaaatatgcatttgaagcatctgttgattgtatggatgtaagtgttcttacatagttagttatatactaatcattttctgttgtggctttctataaaaaataaacagtttatttacaggatttgt

SEQ ID NO: 74 - DNA, 283 bp, Homo sapiens
attagtttgctagtgttgcagtgtcttgtctcgaaacagttcaaggcatcaaatttagaactataaacttcatcgtttgctgtacatcatgaaactgaggtcaagtagaggaaacgtgaaaggtgtcagacggagaggaaagaatgatgcggtgtgcgaatagaaaattaaaaccttgccttgattgttgcccactcaaaatgatatttgccagatgctagtttctgggtcttttaaatttgctgtattaactactgtcttaacaagctcctcctaataaacc

SEQ ID NO: 75 - DNA, 298 bp, Homo sapiens
aagggcaacagttatcacagttcatacacacctttcatgtcctgtctcactcactcctcacagccatcctaggagatacatattgttttcatcctgcatttacagaaaaagaaatgaaaacagagagcttaaataatttgccacagtaatgtcgaaactaggcctttgaaccaaggcagtctagggtaaaatatagtttcaaagtatgaataagaattggtatttgtgttatctttgagtaagaaactgtccgatatgaatcacaacgtgggtgaatgtagtattttcctgaagtgtg

SEQ ID NO: 76 - DNA, 241 bp, Homo sapiens
atgaaagactgtacaaagtagaagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaaataaaaatgaggtactctcctggaaatattaagaaagactatctaaatgttgaaagaccaa

SEQ ID NO: 77 - DNA, 183 bp, Homo sapiens
gctatttttgaggttcgtgcctgttgtagaccacagtcacacactgctgtagtcttcccccatcctcattcccagctgcctcctcctactgtttccctctatcaaaaagcctccttggcgcaggttccctgagctgtgggattctgcactggtgctttggattccctgatatgttccttcaaa

SEQ ID NO: 78 - DNA, 62 bp, Homo sapiens
ttttgtatttttaggtgttcttgaactcctgatgtcaggtgattctcctagctccaaatgtt

SEQ ID NO: 79 - DNA, 264 bp, Homo sapiens
attttcactacaaccctgtaaggaggcttgagaaagaagatgacattcccaaaggcacatttgggcaagcaggaacttgggcaagtattttaacatctttaaacctcagtgaattcatttttttaaaaagaaaaaaatttgttgagcaccgctgtaagcccagtgctgtactaggggctgaagacaatgcatcaaacaggtcacacggagacagggttcctgccccaggaaatttaaagtccagcagggaagatggacattcat

SEQ ID NO: 80 - DNA, 289 bp, Homo sapiens
tcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctca

SEQ ID NO: 81 - DNA, 56 bp, Homo sapiens
gccatgttggtactagttattaatcatatctaaccaactgtaggtgttctttcctg

SEQ ID NO: 82 - DNA, 274 bp, Homo sapiens
atatggtacgttttaaccttgaaagttttgcaatgatgaaagcagtatttgtacaaatgaaaagcagaattctcttttatatggtttatactgttgatcagaaatgttgattgtgcattgagtattaaaaaattagatgtatattattcattgttctttactcatgagtaccttataataataataatgtattctttgttaacaatgccatgttggtactagttattaatcatatctaaccaactgtaggtgttctttcctgataactttttta

SEQ ID NO: 83 - DNA, 101 bp, Homo sapiens
ccaactggcacaattcaattcctactgtacccatcatgcacagatggctgaagtattgagaacgctccagtgaccgggaggcaatagtctgtctctctgtc

SEQ ID NO: 84 - DNA, 261 bp, Homo sapiens
ttcttctctgctgagcctgcaggcccgtcctgcctgcctggggtgcccgggagacgcgggcctgctccggagactgctgactgccggtcctgttagtcaggtgtcagccctgtctctgccgaagagactcttctctttattttaaattaaaccctcagagcaccaccaaagcatcacttttctccctccattggtgttctcattctttgatgttacttgtttgaacaccactattagtagttggagatttgttcctgagaa

SEQ ID NO: 85 - DNA, 277 bp, Homo sapiens
aagtcatttttcagggtccttcaggaagtcatccagagttataatggcccattatttaatggtcagagtttacttaggctttcactacttccactgcccacttgaaacagggaaaaatattttccccccgcgctgtgagtgtgctatttagagctgaccacaagcggggggaagagaggatggctcggatgctgcatttccactgagaacacaaggctggcaaagcttgtctgctgcccagcaagcacttcaggctcacaccattttaggttcactt

SEQ ID NO: 86 - DNA, 279 bp, Homo sapiens
tacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaacatatatgtgaggatttttattgattggggatcttggagtttttcattgtcgctattgatttttacttcaatgggctcttccaacaaggaagaagcttgctggtagcacttgctac

SEQ ID NO: 87 - DNA, 226 bp, Homo sapiens
  misc_feature (120)..(120): n is a, c, g, or t
atgctatatgctgtatcccacctttctctgaatgttacattttctcccctatcccaggctgcatctaagaaaactcaaagggaatatgctatctatcttttccgagcaatgaaagctctngggttttttccttgcttttcagggcacnatacttctctttcttcctggttagacaggataagttctgagtcccntggtatcatcagcttacttcttctctgttaaa

SEQ ID NO: 88 - DNA, 241 bp, Homo sapiens
  misc_feature (119)..(120): n is a, c, g, or t
gcacagcagcaagacagattgccatggagcatgttgtgcccaactagggacagcgcagatagattctgtaatttgcctaacaatgtctataggatgatcccatttgtcaaaaaaaaaanngaactgggctttattgatgtcacctaaatgcacctaaacttcttttttgccccatgctcttctgtactcttgatctttccccaaatttttaaaaacatgacactcattcccttatttttcc

SEQ ID NO: 89 - DNA, 295 bp, Homo sapiens
acatcctttaactcttcctacagaaatctaagagagaaatgaaacaaaagtttgcacagttctagacacgataaatacatgtgaaatcacacaactcagaaaatgtcccttaaattaattgagccattggtacttgtgaattagaagagacatctatgttctgatccactgttgaaagctgtacaatgttacctatttatttgcagacatcctttggaaacaaataggtagatttgcaacaaataaagagtggagtacagctgctgacattaccttgtatattcatgcctttatg

SEQ ID NO: 90 - DNA, 175 bp, Homo sapiens
tggtgaatttaaagactcactctccataaatgctacgaatattaaacacttcaaaaactgcacctccatcagtggcgatctccacatcctgccggtggcatttaggggtgactccttcacacatactccccctctggatccacaggaactggatattctgaaaaccgtaaaggaa

SEQ ID NO: 91 - DNA, 262 bp, Homo sapiens
cactttgcagccttgagaggtgcagaagagacaccgaggggttcaccaccagagccaccattgtcagagaggcgtccagctgtgtccacctgggactctgccttcagggcttcttgcctggctgggagctgcacaggcagactcctgggacggtgtgccgacagctctgggcacccccttctaggatctgattcctgaggaatcacaatgtggatttcacaatcacttccagtgtcttttgccaacctctgtgaacagatgt

SEQ ID NO: 92 - DNA, 252 bp, Homo sapiens
gataaacacatgaccgagcctgcacaagctctttgttgtgtctggttgtttgctgtacctctgttgtaagaatgaatctgcaaaatttctagcttatgaagcaaatcacggacatacacatctgtatgtgtgagtgttcatgatgtgtgtacatctgtgtatgtgtgtgtgtgtatgtgtgtgtttgtgacagatttgatccctgttctctctgctggctctatcttgacctgtgaaacgtatatttaacta

SEQ ID NO: 93 - DNA, 293 bp, Homo sapiens
aatgtgaaactgctccatgaaccccaaagaattatgcacatagatgcgatcattaagatgcgaagccatcgagttaccacctggcatgcttaaactgtaaagagtgggtcaaagtaaactgaattggaaaatccaaagttatgcagaaaaacaataaaggagatagtaaaaagggttaacgagccagtccaggggaagcgaagaagacaaaaagagtccttttctgggccaagtttgataaattaggcctcccgaccctttgctctgttgctttatcaactctactcggcaat

SEQ ID NO: 94 - DNA, 242 bp, Homo sapiens
agttaatatctgttttatgtgcccccagcatgtgttgaacatcaaacagtaccagggactttaaatatacccacggacaaagaaataattcataatgatgtttgttgaatttagttgcaatcaataaaaagtgcagtttgtgaatgctctgaggttcttgatattgatgtaaggctttgaacgacaaatgaggaccaaacataaataggaaagtaaaactgaaggatagaggccaaggccat

SEQ ID NO: 95 - DNA, 231 bp, Homo sapiens
cccggccgggcctgtgttgtgcaatgctgcacatcacaacaggagggtagggggacaaaagagcacaggtcctggcagctgccacagtctccaggggcttttgcgtttctctccagatttctaaggttaacatggggattagctgttttgcaatgaataaaaggtaacattgcctggaatgttgcttaaagacacttttttaaagctagttgattgttaagctgttgctac

SEQ ID NO: 96 - DNA, 263 bp, Homo sapiens
gcagtgggaatgactctgccatgcaccgtgtccccggccgggcctgtgttgtgcaatgctgcacatcacaacaggagggtagggggacaaaagagcacaggtcctggcagctgccacagtctccaggggcttttgcgtttctctccagatttctaaggttaacatggggattagctgttttgcaatgaataaaaggtaacattgcctggaatgttgcttaaagacacttttttaaagctagttgattgttaagctgttgctac

SEQ ID NO: 97 - DNA, 125 bp, Homo sapiens
actgcacctacgggtcctaataaatcttcactgtctgactttagtctcccactaaaactgcatttcctttctacaatttcaatttctccctttgcttcaaataaagtcctgacactattcatttg

SEQ ID NO: 98 - DNA, 146 bp, Homo sapiens
ggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaa

SEQ ID NO: 99 - DNA, 287 bp, Homo sapiens
gaaacctgcagggactccatgctgccagccttctccgtaattagcatggccccagtccatgcttctagccttggttccttctgcccctctgtttgaaattctagagccagctgtgggacaattatctgtgtcaaaagccagatgtgaaaacatctcaataacaaactggctgctttgttcaatgctagaacaacgcctgtcacagagtagaaactcaaaaatatttgctgagtgaatgaacaaatgaataaatgcataataaataattaaccaccaatccaacatcc

SEQ ID NO: 100 - DNA, 188 bp, Homo sapiens
ttgactttcataagtactctagttatgagcttatttaacatttgggttttagtaataggggtatgtgttgagaaaatttcaaagttttagaatatggttcacccacatgttgcttccctgtaaatataatttttaaaaccagattctgggccgggcatggtggctcacctctataatcccaaaacgtt

SEQ ID NO: 101 - DNA, 242 bp, Homo sapiens
agattttgagctatcatctctgcacatgcttagtgagaagattacacaacatttttaagaatttgagattttatattgtcagttaaccactttcattattcattcacctcaggacatgcagaaatatttcagtcagaactgggaaacagaaggacctacattctgctgtcacttatgtgtcaagaagcagatgatcgatgaggcaggtcagttgtaagtgagtcacattgtagcattaaatt

SEQ ID NO: 102 - DNA, 167 bp, Homo sapiens
gaatgttgtagttacctactgagtaggcggcgatttttgtatgttatgaacatgcagttcattattttgtggttctattttactttgtacttgtgtttgcttaaacaaagtgactgtttggcttataaacacattgaatgcgctttattgcccatgggatatgtggt

SEQ ID NO: 103 - DNA, 67 bp, Homo sapiens
gtagctgcgattgggtatgtgtttcctgggttaggggaaaggactctgccctattgagggctgtgag

SEQ ID NO: 104 - DNA, 162 bp, Homo sapiens
actcagtcgggctcccaggacctgaaggccctcaataccagctaccagtcccagctcatcaaacccagccgcatgcgcaagtaccccaaccggcccagcaagacgcccccccacgaacgcccttacgcttgcccagtagagtcctgtgatcgccgcttctcc

SEQ ID NO: 105 - DNA, 271 bp, Homo sapiens
ttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttg

SEQ ID NO: 106 - DNA, 245 bp, Homo sapiens
gggttcaattacagaactgttatcaaaatgcgttggtttatgcacatccgtgttttggacatggtgattccaagagactcttaataaaacttttcaaagtagatgagagacagtttttccctcacatgctgtggcaatattaatctatgttttcatgttccactggactttgtaattgaattttaaggaatgcatacagggcttcatatttatatataaaatatccatatccagtgttgaaagaa

SEQ ID NO: 107 - DNA, 261 bp, Homo sapiens
gaagagcattttaaacaactggaattaagctcaaatacattaccagtggttgaagaattcacatcaataattttttgaatttaggaataaaatggagaagtcaaggaaaggaaaatattatacacaggctagcaatagttaaaatacaattattaaagccagagctagacaaaattatggcaatgagatgtgtaacaaaaccactctgtaacttcatcatgttcgtttaaaagacctgaatgattccaaaatccttagacc

SEQ ID NO: 108 - DNA, 103 bp, Homo sapiens
ttgctcctaacttgctcttggacaggaaccagggaaaatgtgtagagggcatggtggagaggctagagatcctgatgattggtctcgtctggcgctccatgga

SEQ ID NO: 109 - DNA, 294 bp, Homo sapiens
ttcatccagcgctgtgcagtagcccagctgcgtgtctgccgggaggggctgccaagtgccctgcctactggctgcttcccgaatccctgccattccacgcacaaacacatccacacactctctctgcctagttcacacactgagccactcgcacatgcgagcacattccttccttccttctcactctctcggcccttgacttctacaagcccatggaacatttctggaaagacgttcttgatccagcagggtaggcttgttttgatttctctctctgtagctttagcattttga

SEQ ID NO: 110 - DNA, 217 bp, Homo sapiens
tcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacggg

SEQ ID NO: 111 - DNA, 226 bp, Homo sapiens
ggcttcctgaagcttagatttccagcttgtcaccttcaaggttaccttgtgaataggacttttttgagctatttctatccagttgactatggattttgcctgttgctttgtttccaccaactctccctgaagatgaggcgcacagacagacaactcacaggcaagaacagcctggtccatcttgaaagattctcaagactattctccacaagataattgtctactt

SEQ ID NO: 112 - DNA, 265 bp, Homo sapiens
aatctgagatctatgcacccaggaagcctgacacattattgtggtgtctcaattcttttttttttaattagaaaaattgtatcaaattgcattgggtgagagcaaaaataaactgaagttggttgagctttggaagactacaagccactgtaatatttaagatttcttgacctccagaactaacatttgtcctgtcagagaaaataattactcctgttgagaatacatgcattaaagtaagatgttcactactctatatgatcac

SEQ ID NO: 113 - DNA, 269 bp, Homo sapiens
gcatcataacataagcgctttcccccttctcgtcactatcatttgtatcaaccaaagaactgatctctggtatcctcgaaggaatgctgtggggatattcttcatctctgttcatggtacatcagcaatttgtggggaaaagatggactatataacacaatgatctgcctaaaagaaactgtctctacttatagggggctgagcaaaccttagagcatctgcggatgctcgtcattatcttcaaaagtccccaagagtttttctccata

SEQ ID NO: 114 - DNA, 235 bp, Homo sapiens
  misc_feature (107)..(123): n is a, c, g, or t
tggctttccggtcatgggttccagttaattcatgcctcccatggacctatggagagcagcaagttgatcttagttaagtctccctatatgagggataagttcctgannnnnnnnnnnnnnnnngtgttacaaaagaaagccctccctccctgaacttgcagtaaggtcagcttcaggacctgttccagtgggcactgtacttggatcttcccggcgtgtgtgtgccttacacagg

SEQ ID NO: 115 - DNA, 159 bp, Homo sapiens
ttttgagatgaatgataatagcgatacaacttaccaaaacctttgggatacagcaaaagcagtgtcaagaggaaagttcatagcattaaatacctacatcaaaaagtttgaaagagcacaaatagacaatttaaggtcacacctcagagaactagggaa

SEQ ID NO: 116 - DNA, 198 bp, Homo sapiens
ttgagatgaatgataatagcgatacaacttaccaaaacctttgggatacagcaaaagcagtgtcaagaggaaagttcatagcattaaatacctacatcaaaaagtttgaaagagcacaaatagacaatttaaggtcacacctcagagaactagggaaacaagaacaaatcaaacccaaacccagcagaaggaaagaaa

SEQ ID NO: 117 - DNA, 292 bp, Homo sapiens
ggaggtaagccagcctgaagatattgatgacactggatggccttaagtttccattttgactggcatgtaatccagccaaagattcccacagaaatccgaggttccaaatggtaaatccgtggtgacattggggtgaccgagaaatgaactctgtgcagaaaggaatggtaacaacacttgtctcgtacaactttggctctgggtaggggaaaggaaaacagcttcctagagaaatgttaaccacaagctagccctcatgagagatttcagctaggatggatgctatctgtgt

SEQ ID NO: 118 - DNA, 277 bp, Homo sapiens
actgggggcaggagtgtcatcttttgggcagggcaatcctggggctaaatgaggtacaggggaatggactctcccctactgcacccctgggagaggaagccaggcaccgatagagcacccagccccacccctgtaaatggaatttaccagatgaagggaatgaagtccctcactgagcctcagatttcctcacctgtgaaatgggctgaggcaggaaatgggaaaaagtgttagtgcttccaggcggcactgacagcctcagtaacaataaaaacaa

SEQ ID NO: 119 - DNA, 294 bp, Homo sapiens
gattaagaacagttttttcaacaaatagtgttgggacaatgggtgtccacatgcaaaagaataaagttgtccccttaccttacaccatctccaaaaattaactcaaaatatgtcaaagacataaacgtaagagctaaaactgtaaaactcctagaataaaacataggagtaaatcttcatgaccttggattaggccattgtgtcttaaatataacaccaaaagaataagtaataaaaaaatagataaattgaactccatcaaaattaaaagcctttgtgcttcataggacacca

SEQ ID NO: 120 - DNA, 229 bp, Homo sapiens
agacgccgggaacgcaggccgctttattcctctgtacttagatcaacttgaccgtactaaaatccctttctgttttaaccagttaaacatgcctcttctacagctccatttttgatagttggataatccagtatctgccaagagcatgttgggtctcccgtgactgctgcctcatcgataccccatttagctccagaaagcaaagaaaactcgagtaacacttgtttga

SEQ ID NO: 121 - DNA, 135 bp, Homo sapiens
ggctgaggttgggtttgtcatcacagagggggtgggcctggaaagggtccttcccaagctgccccggctccggcggcccgggccggcagcctctgccagccagcgtcctcacggcctccccctcgcctgtttctt

SEQ ID NO: 122 - DNA, 107 bp, Homo sapiens
agcaagtgtagacaccttcgagggcagagatcgggagatttaagatgttacagcatatttttttttcttgttttacagtattcaattttgtgttgattcagctaaat

SEQ ID NO: 123 - DNA, 247 bp, Homo sapiens
ggctgaggttgggtttgtcatcacagagggggtgggcctggaaagggtccttcccaagctgccccggctccggcggcccgggccggcagcctctgccagccagcgtcctcacggcctccccctcgcctgtttcttttgaaagcaagtgtagacaccttcgagggcagagatcgggagatttaagatgttacagcatatttttttttcttgttttacagtattcaattttgtgttgattcagctaaat

SEQ ID NO: 124 - DNA, 67 bp, Homo sapiens
acgcctgtgtccccgcgttctgagaagtcctctgtcttcgtgtcactaggtccagaaagtcgcgccg

SEQ ID NO: 125 - DNA, 142 bp, Homo sapiens
cagggccgaggaataagcgacaattctggtttttctcccctggccgtcgttcgccagcctccttcattttcctgagttcccgctgaagtatatactacctatgagtccaattaacatgagtattatgctagttctatcctac

SEQ ID NO: 126 - DNA, 279 bp, Homo sapiens
ctttcgccactcacggaccttgaggccagttgacggcccttctccccacgcctgtgtccccgcgttctgagaagtcctctgtcttcgtgtcactaggtccagaaagtcgcgccgggcagaggcgcaggcggggccggcagggccgaggaataagcgacaattctggtttttctcccctggccgtcgttcgccagcctccttcattttcctgagttcccgctgaagtatatactacctatgagtccaattaacatgagtattatgctagttctatcctac

SEQ ID NO: 127 - DNA, 263 bp, Homo sapiens
ccatgggagaactggatgttcaccaggggtcagcattggccttgaagtgtggagaagggtcatcttggcagaggtggcaaggtggtgagcccctggggctgagcacaggtgcgtctggtgagaggggcctggccatgaccgcagtgactgctcttcactgtcacctcctttgctcctcaggccacctgcgcagagggtgtgatccttgcatgactttgccattgaggaaatgcaagggtagaaagtgcagtctcgtcggccgc

SEQ ID NO: 128 - DNA, 252 bp, Homo sapiens
gggtcagaaggttagcctgcaggtgtgggatcacagaggtgtcctagtgatggagcttgtatgctctatgggttaaaaacagacgctaaggagataaactatacacagaagaagcttaatgggctgtgcacagtggcttgcacttgcaatcccagctctttcggaggccgaggtaggaggctaggagttcgagaacagcctggggcaacatagtgagacaccccccaccccaacccatctcattatgttgag

SEQ ID NO: 129 - DNA, 252 bp, Homo sapiens; identical to SEQ ID NO: 128

SEQ ID NO: 130 - DNA, 297 bp, Homo sapiens
agagctgaggctttggtacccccaaacccccaatatttttggactggcagactcaaggggctggaatctcatgattccatgcccgagtccgcccatccctgaccatggttttggctctcccaccccgccgttccctgcgcttcatctcatgaggatttctttatgaggcaaatttatattttttaatatcggggggtggaccacgccgccctccatccgtgctgcatgaaaaacattccacgtgccccttgtcgcgcgtctcccatcctgatcccagacccattccttagctattta

SEQ ID NO: 131 - DNA, 205 bp, Homo sapiens
gtgagactgagggatcgtagatttttacaatctgtatctttgacaattctgggtgcgagtgtgagagtgtgagcagggcttgctcctgccaaccacaattcaatgaatccccgacccccctaccccatgctgtacttgtggttctctttttgtattttgcatctgaccccggggggctgggacagattggcaatgggccgtcccc

SEQ ID NO: 132 - DNA, 228 bp, Homo sapiens
ttctacccttaacactctggaaagcctgtgaaatgaaattattccacctcctgccctagccacccacagctctcctggtgctggtggcatcccccaaaacccactcccttcctacgtcctcccttggtctgagagttccctgctgtatgcctgcagggtgagctgttactccttgagggaacaagggaattgtcaactttccttctctactttttctcttccccggga

SEQ ID NO: 133 - DNA, 252 bp, Homo sapiens
  misc_feature (86)..(144): n is a, c, g, or t
agtgaccagccttccgatcccctgaactcgccctccctcctcgctctgtgaactctttagacacacaaaacaaacaaacacatggnnnnnnnnnnnnnnnnnnnnnnnnnnnnnnnnnnnnnnnnnnnnnnnnnnnnnnnnnnncaaagtgggtgtgtggcctcccgggctcctccgtctgaccctctgcggccactgcgccactgccatcggacaggaggattccttgtgttttgtcctgcctcttgtttc

SEQ ID NO: 134 - DNA, 286 bp, Homo sapiens
  misc_feature (86)..(144): n is a, c, g, or t
agtgaccagccttccgatcccctgaactcgccctccctcctcgctctgtgaactctttagacacacaaaacaaacaaacacatggnnnnnnnnnnnnnnnnnnnnnnnnnnnnnnnnnnnnnnnnnnnnnnnnnnnnnnnnnnncaaagtgggtgtgtggcctcccgggctcctccgtctgaccctctgcggccactgcgccactgccatcggacaggaggattccttgtgttttgtcctgcctcttgtttctgtgccccggcgaggccggagagctggtgacttt

SEQ ID NO: 135 - DNA, 234 bp, Homo sapiens
gaaaaagtaaggggctacagctgggcgcagtggcttgcgcctgtaatcccagcattttgggaggccaagatgggcggatcacttgaggtcaggagtttgagaccagccttgccaagatggtgaaaccctgtctttacaaaaaatacaaaaattagcctggtgtggtggtgcacacctgtaatcccaggctacttgagaggttgaggcaggagaatcactttaacttaggaggca

SEQ ID NO: 136 - DNA, 238 bp, Homo sapiens
attagagtcctatattcaactaaagttacaacttccataacttctaaaaagtggggaaccagagatctacaggtaaaacctggtgaatctctagaagttatacaaaccacagatgacacaaaagttctctgcagaaatgaagaagggaaatatggttatgtccttcggagttacctagcggacaatgatggagagatctatgatgatattgctgatggctgcatctatgacaatgact

SEQ ID NO: 137 - DNA, 260 bp, Homo sapiens
ttcaacacagctgtggtcttcctctgaatattagcagaagtttcttattcaaaggcctcctcccagaagaagtcagtgggaagagatggccaggggaggaagtgggtttattttctgttgctattgatagtcattgtattactagaaatgaactgttgatgaatagaatatattcaggacaatttggtcaattccaatgcaagtacggaaactgagttgtcccaaattgatgtgacagtcaggctgtttcatcttttttg

SEQ ID NO: 138 - DNA, 177 bp, Homo sapiens
gacttgcattgcagtctgacagtaattttttttctgattgagaattatgtaaattcaatacaatgtcagtttttaaaagtcaaagttagatcaagagaatatttcagagttttggtttacacatcaagaaacagacacacatacctaggaaagatttacacaatagataatcatctt

SEQ ID NO: 139 - DNA, 114 bp, Homo sapiens
attttttcaaactttcatatagagttataagattatgatgctggtatctggtaaaatgtacatcccagtagtccaatagtttaaatgtttattgcttcctttaagagattataa

SEQ ID NO: 140 - DNA, 153 bp, Homo sapiens
agtgagctccagcacgcccagaggactgttaataacgatgatccatgtgttttactctaaagtgctaaatatgggagtttcctttttttactctttgtcactgatgacacaacagaaaagaaactgtagaccttgggacaatcaacatttaaa

SEQ ID NO: 141 - DNA, 178 bp, Homo sapiens
cacaacttaaatagggcattttataacctgaacacaatttatattggacttaattattatgtgtaatatgtttataatcctttagatcttataaatatgtggtataaggaatgccatataatgtgccaaaaatctgagtgcatttaatttaatgcttgcttatagtgctaaagttaaa

SEQ ID NO: 142 - DNA, 223 bp, Homo sapiens
ttccttgcaagttgaaacattattgtgctaggattgctctctagacaagccagaagtgacttattaaactattgaaggaaaaggactcaagaaaaataataaaagaccataaataagggcgaaaacattatcatgtgaaaagaatgtatttcacctgcaagttacaaaaaaatagtttgtgcattgcaaataagcaaagacttggattgactttacattcatc

SEQ ID NO: 143 - DNA, 167 bp, Homo sapiens
attcctgtcattacccattgtaacagagccacaaactaatactatgcaatgttttaccaataatgcaatacaaaagacctcaaaatacctgtgcatttcttgtaggaaaacaacaaaaggtaattatgtgtaattatactagaagttttgtaatctgtatcttatca

SEQ ID NO: 144 - DNA, 234 bp, Homo sapiens
taatcttctctattgtatacttgatttgtccattaatatttctgtccattatttcttgcttttatttttattgttgatttctttctacttactttgagtttaattggtattttttctgactttttgaagtgaaagtctatataattaattttcagcctttctccatttctaatacatatgtttaaacttatatgatttttgtaacattttatcttcattgtataagttttaaca

SEQ ID NO: 145 - DNA, 263 bp, Homo sapiens
gttttggttttgttaatcttctctattgtatacttgatttgtccattaatatttctgtccattatttcttgcttttatttttattgttgatttctttctacttactttgagtttaattggtattttttctgactttttgaagtgaaagtctatataattaattttcagcctttctccatttctaatacatatgtttaaacttatatgatttttgtaacattttatcttcattgtataagttttaacaagtatcttttgtcatt

SEQ ID NO: 146 - DNA, 70 bp, Homo sapiens
ataaagaaggaagagtgtttcatttatatctgaatgaaaatatgaatgactctaagtaattgaattaatt

SEQ ID NO: 147 - DNA, 240 bp, Homo sapiens
ctagccagggagaaagagtgagatcctggctcaaaaaaaccaaataaaacaaaacaaacaaacgaaaaacagaaaggaagactgaaagagaatgaaaagctggggagaggaaataaaaataaagaaggaagagtgtttcatttatatctgaatgaaaatatgaatgactctaagtaattgaattaattaaaatgagccaacttttttttaacaatttacattttatttctatgggaaaaa

SEQ ID NO: 148 - DNA, 222 bp, Homo sapiens
ataacactctatatagagctatgtgagtactaatcacattgaataatagttataaaattattgtatagacatttgctttttaaacagattgtgagttctttgagaaacagcgtggattttacttatctgtgtattcacagagcttagcacagtgcctggtaatgagcaagcatacttgccattacttttccttcccactctctccaacatcacattcacttt

SEQ ID NO: 149 - DNA, 256 bp, Homo sapiens
aaaagtggataatcttgtcttgtactttattttagaggaaaagctgtcagtttttcactgctgaatatgatgttaactatgaactttttatacatgtatttactatgttgaggtaatttccttttactcctggtttaagtgttttttgtttttttttgtttttttttttttttaaatcatggaaggacttgggttttatcaaatgtcttttctgtatctattgagatgaccaatttgtattagtcagcgttcttca

SEQ ID NO: 150 - DNA, 252 bp, Homo sapiens
gtggataatcttgtcttgtactttattttagaggaaaagctgtcagtttttcactgctgaatatgatgttaactatgaactttttatacatgtatttactatgttgaggtaatttccttttactcctggtttaagtgttttttgtttttttttgtttttttttttttttaaatcatggaaggacttgggttttatcaaatgtcttttctgtatctattgagatgaccaatttgtattagtcagcgttcttca

SEQ ID NO: 151 - DNA, 272 bp, Homo sapiens
tcctctcatctgcatttctcagaaatgccctccctgcccagtggtgactttccctcgtcactcctatggagttctacctggagcccagccatgtgtggaactgtgaagtttactcctctgtaaagatggtttaaagaaagtcagcttctgaaatgtaacaatgctaacccttgctggaaccctgtaagaaatagccctgctgatagttttctaggtttatcatgtttgatttttacactgaaaaataaaaaaatcctggtatgtttgaaatt

SEQ ID NO: 152 - DNA, 213 bp, Homo sapiens
accatcgcacttgagccagtcgattccatcccatatctcatatgaagactttaaaaaggagaatttctagccctcaacaccttgtaaggactttagtttctggctcttttttcaaaggagattatattcaaaaggaatactaaaaaattagccaggcataatggcaaacactggtagtcccagctattttgaaggctgaggtgacaggctcac

SEQ ID NO: 153 - DNA, 213 bp, Homo sapiens; identical to SEQ ID NO: 152

SEQ ID NO: 154 - DNA, 230 bp, Homo sapiens
aaggtatttggtcattatcttctacagcagtggaatgagtggtcccggagatgtgctatatgaaacattctttctgagatatatcaaccacacgtggaaaagcctttcagtcatacatgcaaatccacaaagaggaagagctgaccagctgaccttgctgggaagcctcacccttctgcccttcacaggctgaagggttaagatctaatctccctaatctaaatgacagt

SEQ ID NO: 155 - DNA, 249 bp, Homo sapiens
  misc_feature (69)..(71): n is a, c, g, or t
aggttttatgaaattccatggcaacagtcccaacatgtttgagacttcagctaaaggaatggatgtatnnnggngtgtagtcttcagtatatcactgtatttccgtaatactagactcnaagntatgcnagatngnttattcccttngtgaannnggagttgctcattacgttcttgaaatatcgcacatcctgttggttcttcaaaggaagcctttccaccagattagtgttcaagtctttgcagagg

SEQ ID NO: 156 - DNA, 292 bp, Homo sapiens
cattctccaagcatcagatccatttcctatcacaacatttttaaaaaatgtcatctgatggcacttctgcttctgtcctttaccttcccatctccagtgaaaagctgagctgctttgggctaaaccagttgtctatagaagaaaatctatgccagaagaactcatggttttaaatatagaccatcatcgaaactccagaaatttatccactgtggatgatgacatcgctttcctttggtcaaggttggcagagcaagggtataaagggggaaattgtttggcagcaccaaca

SEQ ID NO: 157 - DNA, 207 bp, Homo sapiens
ataaaaagcgggcacgggcccgtgacatccccacccttggaggctgtcttctcaggctctgccctgccctagctccacaccctctcccaggacccatcacgcctgtgcagtggcccccacagaaagactgagctcaaggtgggaaccacgtctgctaacttggagccccagtgccaagcacagtgcctgcatgtatttatccaataa

SEQ ID NO: 158 - DNA, 119 bp, Homo sapiens
aaaaagaggggctgggctgggcgcggtggttcacgcctgtaatcccagcactttgggaggccaaggagggtggatcacctgaggtcaggagttagaggccagcctggcgaaaccccatc

SEQ ID NO: 159 - DNA, 132 bp, Homo sapiens
ttctagggaaggtgttctgggggccgggctctctccagctgtgggaggcctgctccctctggggggcaccctgggcagggtgggggggccttgggaggcgcttcttgccaaatgcagacgaggggtgagcct

SEQ ID NO: 160 - DNA, 132 bp, Homo sapiens
gtgagcctgccagcgtttgcgacgtccccgcacgacaggctcatactttctgaggatcgtgcatagcataggacgtctgaacctttgtacaaatgtgtagatgacatcttgctacagcttttatttgtgaat

SEQ ID NO: 161 - DNA, 246 bp, Homo sapiens
tgtggacagtggacgtctgtcacccaagagagttgtgggagacaagatcacagctatgagcacctcgcacggtgtccaggatgcacagcacaatccatgatgcgttttctccccttacgcactttgaaacccatgctagaaaagtgaatacatctgactgtgctccactccaacctccagcctggatgtccctgtctgggccctttttctgttttttattctatgttcagcaccactggcaccaaa

SEQ ID NO: 162 - DNA, 185 bp, Homo sapiens
gaggaccggctgcagacctcactctgagtggcaggcagagaaccaaagctgcttcgctgctctccagggagaccctcctgggatgggcctgagaggccggggctcagggaaggggctgggatcggaacttcctgctcttgtttctggacaactttccccttctgctttaaaggttgtcgattatt

SEQ ID NO: 163 - DNA, 234 bp, Homo sapiens
ggcctcacacaaggagtgtttggcttacagtgaattgtccggggggttttgcccacctcctcctcatctccgtattcttcagcttcatccaaaactgacttagaagcctcccttgaccctcacctgactattcacaggttatagcactttatgtttttcagttctgttattttaattggtgcctctgtttgtgatctttaagaacataaaattctggcaagtaactatttgcta

SEQ ID NO: 164 - DNA, 223 bp, Homo sapiens
aagattcctgtgtactggtttacatttgtgtgagtggcatactcaagtctgctgtgcctgtcgtcgtgactgtcagtattctcgctattttatagtcgtgccatgttgttactcacagcgttctgacatactttcatgtggtaggttctttctcaggaactcagtttaactattatttattgatatatcattacctttgaaaagcttctactggcacaattta

SEQ ID NO: 165 - DNA, 256 bp, Homo sapiens
ttttggctttttgcatatctagtataataggaagtgtgagcaaggtgatgatgtggctgtgatttccgacgtctggtgtgtggagagtactgcatgagcagagttcttctattataaaattaccatatcttgccattcacagcaggtcctgtgaatacgtttttactgagtgtctttaaatgaggtgttctagacagtgtgctgataatgtattgtgcgggtgacctcttcgctatgattgtatctcttactgttt

SEQ ID NO: 166 - DNA, 216 bp, Homo sapiens
ggatcttattgcacttgggctgttcagaatgtagaaaggacatatttgaggaagtatctatttgagcactgatttactctgtaaaaagcaaaatctctctgtcctaaactaatggaagcgattctcccatgctcatgtgtaatggttttaacgttactcactggagagattggactttctggagttatttaaccactatgttcagtattttaggac

SEQ ID NO: 167 - DNA, 273 bp, Homo sapiens
caaggagctagggatcccttctcaggttctctggatgtgatatccctcagtgaaaactgggaatcatgttaatctcaacgcccgatgtagaatttcatgtcaagtctcaatagttgattttctaaaccacacaccatctataggataatatccccattcattcattcattcattcattcattcattcattcatctgctatgggctaggcactgcccatgcctaaacaaagacataatcttacagagcttagagtctagcaacaccattaccac

SEQ ID NO: 168 - DNA, 252 bp, Homo sapiens
  misc_feature (56)..(56): n is a, c, g, or t
tgagctctttctctccgaagagcggtagcttggccggcccacgttccgctccgctntgcgctctcccctccccggctctctcctggaaggttatttgtagcctctttggcgtgttgggacagcggccctactggggtaggaatgtagctgttcttcccaccacatgggccccttaaagggacagctttgctttttggggggtttcccccttcgggttgcaaggaaaggggatggaactttcctctcagcccc

SEQ ID NO: 169 - DNA, 224 bp, Homo sapiens
aaatgctacaaaaagccccaaagcatataatctctactccttacagtctctagaattaaatgtactcatttagacaacatattaaatgcatattttagccactttagagaaacctcataggcacagagtttccaagattaattttaagaatatcttcacgaacttgaccctcctactccacattgcaacatttccatcagacagcatttcaattccagtattat

SEQ ID NO: 170 - DNA, 259 bp, Homo sapiens
gagaaatttacctgcacaaggctctactctgtgcatcggccggttaaacaatgcattcatcagttatgcttcaccagtttacgacgtatgtacatcgtcaacaaggagatctgctctcgtcttgtctgtaaggaacacgaagctatgaaagatgagctttgccgtcagatggctggtctgccccctaggagactccgtcgctccaattacttccgacttcctccctgtgaaaatgtggatttgcagagacccaatggtc

SEQ ID NO: 171 - DNA, 246 bp, Homo sapiens
catggtaagttttgtagtcctgtaagattctgcaacacagtcaagaattatacaatcctactagcaatatataaggacccaaaatgtcttctgctaagctcagaggctggggctaaagcatgaggactatgccagctatagaacttggactcataattcgctatccaatttttcatgcagttgtctagtcgggaagtaaggttggaaactaagtctcatttactgattcgtttatgggtagtaccg

SEQ ID NO: 172 - DNA, 131 bp, Homo sapiens
tccaaccattgacagctaacccttagacagtatttcttaaaccaatccttttgcaatgtccagcttttacccctactctctactttttcacccaaactgataacatttatctcattttctagcacttaaaa

SEQ ID NO: 173 - DNA, 38 bp, Homo sapiens
gaagacagagccccaccctcagatgcacatgagctggc

SEQ ID NO: 174 - DNA, 207 bp, Homo sapiens
attgaaggatgctgtcttcgtactgggaaagggattttcagccctcagaatcgctccaccttgcagctctccccttctctgtattcctagaaactgacacatgctgaacatcacagcttatttcctcatttttataatgtcccttcacaaacccagtgttttaggagcatgagtgccgtgtgtgtgcgtcctgtcggagccctgtct

SEQ ID NO: 175 - DNA, 256 bp, Homo sapiens
gacacactgccggatccagaggggtggacatcagtggtgggtctgtgatggcggcaaacaacagcagacagcaaatggcagtggtggatggcaagcgaaagctcagctccagccataacaaacacggaccagaagagtgtgcagttgcaagatttaacagagtgaaaacagacgtcccatacaaagggagggaacccaaagggggttgccattgctgggtcgaatgcctgggtttctgtctcaatcactgtccctc

SEQ ID NO: 176 - DNA, 256 bp, Homo sapiens; identical to SEQ ID NO: 175

SEQ ID NO: 177 - DNA, 96 bp, Homo sapiens
aaatgtcttttaaagatggcctgtggttatcttggaaattggtgatttatgctagaaagcttttaatgttggtttattgttgaattcctagaaaag

SEQ ID NO: 178 - DNA, 194 bp, Homo sapiens
tggacactgggcgaattactttttagatctgtagctctgactcctcaggcataaaatgggaataatgcttttacagtttagtggcggaactaaactcccaaaattatttgttatatggatcaagtaataacgtcagtaatgtttttggtacaaagtcattatttaataaaagttattgttccatcttgcttgcc

SEQ ID NO: 179 - DNA, 184 bp, Homo sapiens
aaaacaatcttgtctatttgtcatccagctcaccagttatcaactgacgacctatcatgtatcttctgtacccttaccttattttgaagaaaatcctagacatcaaatcatttcacctataaaaaagtcatcatatataattaaacagctttttaaagaaacataaccacaaaccttttcaaat

SEQ ID NO: 180 - DNA, 178 bp, Homo sapiens
agtggttttttggaagacttaggatattatggtgctacataatttttcctcgatgctctcttcctctcatctttcttgtctcttaaattactttacttccttgcacactttgccatacaagaatgaacatgagcttttcttgtgtagatctgagttgaaatcctgtggacactgggcg

SEQ ID NO: 181 - DNA, 152 bp, Homo sapiens
tggacctgttccaagctctcacgttccacatcacacatgggacatctagtgtcaggctcccagagagcaggaaccaggtgaaatacaagagcacagtcctcccagccggtggcatggggataatcggacaatacaactctccaccctttttt

SEQ ID NO: 182 - DNA, 144 bp, Homo sapiens
gagcatcaccccctgaacatggacttgcagaattccacagaagagaggagactggcctagacagacagccccaggagctgagggcccaacaggctttctaccctggatgctgctcccatgccctgacatgaggcccactacaat

SEQ ID NO: 183 - DNA, 126 bp, Homo sapiens
caaaagtgacttaagtcaggttcccccaaaccagacaccaagacaagaatccatgtgtgtgtgactgaaggaagtgctgggagagccccagctgcagcctggatgtgaactgcaactccaaagtgt

SEQ ID NO: 184 - DNA, 179 bp, Homo sapiens
aggcaagggcactaggctttccagacctcctactaagtcattgatccagcactgccctgccaggacataaatccctggcacctcttgctctctgcaaaggagggcaaagcagcttcaggaggcccttgggagtcctccaaagagagtctagggtacaggtccgaaagtagaagaacaca

SEQ ID NO: 185 - DNA, 116 bp, Homo sapiens
acgtgtacagaagggccatgctgttattactcttacacaaggaggcagccctcgagccacagggtccagctgttggctataatagcctaccggtctctgatgatcaccatgtttct

SEQ ID NO: 186 - DNA, 84 bp, Homo sapiens
gcagcttccggccagagcacgtgtccaggctggccaccggcttgagcaagtccctgcagctgacggagctcacgctgacccagt

SEQ ID NO: 187 - DNA, 299 bp, Homo sapiens
cccttacttacatactagcttccaaggacaggtggaggtagggccagcctggcgggagtggagaagcccagtctgtcctatgtaagggacaaagccaggtctaatggtactgggtagggggcactgccaagacaataagctaggctactgggtccagctactactttggtgggattcaggtgagtctccatgcacttcacatgttacccagtgttcttgttacttccaaggagaaccaagaatggctctgtcacactcgaagccaggtttgatcaataaacacaatggtattccacgtc

SEQ ID NO: 188 - DNA, 119 bp, Homo sapiens
caccagggcacccacttgaacctgcctgcatggctggagcgtagcacttctgaaagcccttcctcaaggcacccccactctccttggggttaccacctcactcgccccagaacatctgg

SEQ ID NO: 189 - DNA, 151 bp, Homo sapiens
gggtctgtaaccagagactggacctggaatcaggactcttggctcaaaagggatttaactccccgggcctcagcttcttcatctgcacagtggatggtcagcctttccttttattgtcattgtggtgattctattatagtaattaccttaa

SEQ ID NO: 190 - DNA, 278 bp, Homo sapiens
cctgccctggaagtaatcttgctgtcctggaatctcctcggggatgaggcagctgccgagctggcccaggtgctgccgaagatgggccggctgaagagagtggacctggagaagaatcagatcacagctttgggggcctggctcctggctgaaggactggcccaggggtctagcatccaagtcatccgcctctggaataaccccattccctgcgacatggcccagcacctgaagagccaggagcccaggctggactttgccttctttgacaaccagcc

SEQ ID NO: 191 - DNA, 266 bp, Homo sapiens
tctttgtacaggaaatattgcccaatgactagtcctcatccatgtagcaccactaattcttccatgcctggaagaaacctggggacttagttaggtagattaatatctggagctcctcgagggaccaaatctccaacttttttttcccctcactagcacctggaatgatgctttgtatgtggcagataagtaaatttggcatgcttatatattctacatctgtaaagtgctgagttttatggagagaggcctttttatgcattaaa

SEQ ID NO: 192 - DNA, 218 bp, Homo sapiens
gtttaagcctggaacttgtaagaaaatgaaaatttaatttttttttctaggacgagctatagaaaagctattgagagtatctagttaatcagtgcagtagttggaaaccttgctggtgtatgtgatgtgcttctgtgcttttgaatgactttatcatctagtctttgtctatttttcctttgatgttcaagtcctagtctataggattggcagtttaa

SEQ ID NO: 193 - DNA, 64 bp, Homo sapiens
gaccttgacgggcaactcggcgctggtgctgctggcggtgcgcgacccgcgcctgcacacgccc

SEQ ID NO: 194 - DNA, 247 bp, Homo sapiens
tcttcgcccttgtcctcctgtgctacctcctgaccttgacgggcaactcggcgctggtgctgctggcggtgcgcgacccgcgcctgcacacgcccatgtactacttcctctgccacctggccttggtagacgcgggcttcactactagcgtggtgccgccgctgctggccaacctgcgcggaccagcgctctggctgccgcgcagccactgcacggcccagctgtgcgcatcgctggctctgggttc

SEQ ID NO: 195 - DNA, 263 bp, Homo sapiens
ggttagttccagagtgcaaattacagaaggaagctacttgtttaaaattccatacacgtttgcagttttttgtacacatttggatactttgaaagatgacagattgttaaatccattcaatggtaaagaaactcaccatttggagattgagtttacttgttaatgaatgactagcccaattatccttataaattgaatatggtgaccaaatgctttgatatcatactactctgcctttgtgggcacatatgtagacactacta

SEQ ID NO: 196 - DNA, 81 bp, Homo sapiens
cactaagttccaattttgttgctgaattgcttctgtgagttcacttttcagttctaaggaagaataatatttgctacatat

SEQ ID NO: 197 - DNA, 204 bp, Homo sapiens
atttgctacatatttcacaggggttcttatgaaggtaaatttaccagattaataaaaatttatgaatattaaaattatcattaataatataaaacacttatttgagattaaattaaatttttcatgagcccctctttggcaggaactctgtttaattctttgtatttatcccagcttcttaaatggtggctgtaacataataaa

SEQ ID NO: 198 - DNA, 195 bp, Homo sapiens
taatcctcaaatatactgtaccattttagatattttttaaacagattaatttggagaagttttattcattacctaattctgtggcaaaaatggtgcctctgatgttgtgatatagtattgtcagtgtgtacatatataaaacctgtgtaaacctctgtccttatgaaccataacaaatgtagctttttaaagtcc

SEQ ID NO: 199 - DNA, 206 bp, Homo sapiens
taatcctcaaatatactgtaccattttagatattttttaaacagattaatttggagaagttttattcattacctaattctgtggcaaaaatggtgcctctgatgttgtgatatagtattgtcagtgtgtacatatataaaacctgtgtaaacctctgtccttatgaaccataacaaatgtagctttttaaagtccattgtattgtt

SEQ ID NO: 200 - DNA, 268 bp, Homo sapiens
accatgttcatcttgtcctccaagttatgggggatcttgtactgacaatctgtgttttccaggagttacgtcaaactacctgtactggtttaaataagtttaccttttcctccaggaaatataatgatttctgggaacatgggcatgtatatatatatatggagagagaattttgcacatattatacatattttgtgctaatcttgttttcctcttagtattcctttgtataaattagtgtttgtctagcatgtttgtttaatccttt

SEQ ID NO: 201 - DNA, 261 bp, Homo sapiens
ggagggaaggcaagattctttccccctccctgctgaagcatgtggtacagaggcaagagcagagcctgagaagcgtcaggtcccacttctgccatgcagctactatgagccctcggggcctcctcctgggcctcagcttgcccagatacatacctaaatatatatatatatatatgagggagaacgcctcacccagattttatcatgctggaaagagtgtatgtatgtgaagatgcttggtcaacttgtacccagtgaaca

SEQ ID NO: 202 - DNA, 237 bp, Homo sapiens
atcccagacacagaagtggagtcaaggctgggcacctctgggacagcaaaaaaaactgcagaatgcatccctaaaactcacgaaagaggcagtaaggaacccagcacaaaagaaccctcaacccatataccaccactggattccaagggagccaactcggtctgagagaggaggaggtatcttgggatcaagactgcagtttgggaatgcatggacaccggatttgtttcttattcc

SEQ ID NO: 203 - DNA, 217 bp, Homo sapiens
aacaaagcagccacagtttcagacaaatgttcagtgtgagtgaggaaaacatgttcagtgaggaaaaaacattcagacaaatgttcagtgaggaaaaaaaggggaagttggggataggcagatgttgacttgaggagttaatgtgatctttggggagatacatcttatagagttagaaatagaatctgaatttctaaagggagattctggcttggga

SEQ ID NO: 204 - DNA, 298 bp, Homo sapiens
aacaaagcagccacagtttcagacaaatgttcagtgtgagtgaggaaaacatgttcagtgaggaaaaaacattcagacaaatgttcagtgaggaaaaaaaggggaagttggggataggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagcccctgg

SEQ ID NO: 205 - DNA, 180 bp, Homo sapiens
tctttaaattagaggatgctgtgccattgagtactttaagttaatatgaggttctggttcaaggaaaacttacgttggatctgaaccaatgagcagatattttgatatgtgccactcttgcatatacatctcagtcctaactaaagattctagtggcatccaggacctttagggaggcat

SEQ ID NO: 206 - DNA, 180 bp, Homo sapiens; identical to SEQ ID NO: 205

SEQ ID NO: 207 - DNA, 294 bp, Homo sapiens
ctagatttgccagggaaccagaatttatggatgaactgattgcttatattttagtcagggtttataaatgtagatggtcaaatttacattgcctagtgatggaaaattcaactttttttgatttttttttccaatattaaaaaaggctctgtatgcatggtggggctatgtaagtactctttaaaactatggccctattaatcttacaagtgttacttatgggtcaagcaatgtaaactgtataaatgtaaaaacaacccctccacacacataacccctggaatatatggtaaa

SEQ ID NO: 208 - DNA, 154 bp, Homo sapiens
cactgcgtctggcaataatgtaactttgaagcttaaaaattaatcccagtttgtagcaataacagaagactatctacaacggaagaaagaagcaactgccttacagttctgtaaagaattggcaagaaaataaagcctatagttgccgaaaaat

SEQ ID NO: 209 - DNA, 145 bp, Homo sapiens
gggctcccggcgctgagcggagacgagtttttctccccgcagaggaggatcctggaaggacgcggagctggggcttgggggatgcagtggcctggacgaggtgactccaacccgggggagcagagtccgtcacgccggcagggac

SEQ ID NO: 210 - DNA, 251 bp, Homo sapiens
ggttttccagtcctcaagggaatactgaagatgctgactgaaggggattggatgttgattttagaagatggagaactccagccacctttgtaaagcactagtgtttgtcatttatgtaagtcaggtcggctcaggtcttgatagtccgtcttggtgtgaggcatgcctgtcacgatgacctagctaacactgtgcatcttattgtgaggccagcttgtcccctcgaaccctctttggccaggtaaacattg

SEQ ID NO: 211 - DNA, 289 bp, Homo sapiens
tgccagaacagtttgtacagacgtatgcttattttaaaattttatctcttattcagtaaaaaacaacttctttgtaatcgttatgtgtgtatatgtatgtgtgtatgggtgtgtgtttgtgtgagagacagagaaagagagagaattctttcaagtgaatctaaaagcttttgcttttcctttgtttttatgaagaaaaaatacattttatattagaagtgttaacttagcttgaaggatctgtttttaaaaatcataaactgtgtgcagactcaataaaatcatgtac

SEQ ID NO: 212 - DNA, 264 bp, Homo sapiens
attgcatatgcatagttcccatgttaaatcccattcataactttcattaaagcatttactttgaatttctccaatgcttagaatgtttttaccaggaatggatgtcgctaatcataataaaattcaaccattattttttttttgtttataatacattgtgttatatgttcaaatatgaaatgtgtatgcacctattgaaatatgtttaatgcatttattaacatttgcaggacacttttacaggccccaattatccaatagttt

SEQ ID NO: 213 - DNA, 217 bp, Homo sapiens
gtagaaaaggctatgctttcaatctcctacacaaattttacatctggaatgatctgaaggttcttcaaagacattcaaaattaggcttttttatgtcctgttttaagtgaaaatatttattcttctaagggtccattttatttgtattcattcttttgtaaacctctttacatttctctttacattttattctttgcccaaatcaaaagtgattcct

SEQ ID NO: 214 - DNA, 288 bp, Homo sapiens
gaggtagttactatcctattactgtacttagttggctatgctggcatgtcattatgggtaaaagtttgatggatttatttgtgagttatttggttatgaaaatctagagattgaagtttttcattagaaaataacacacataacaagtctatgatcattttgcatttctgtaatcacagaatagttctgcaatatttcatgtatattggaattgaagttcaattgaattttatctgtatttagtaaaaattaactttagctttgatactaatgaataaagctgggttt

SEQ ID NO: 215 - DNA, 231 bp, Homo sapiens
gcaaataaattcatacatagtacatacaaaataagagaaaaaattaaattgcagatggttaaatatcacatcacttaactgatgttactgaaaatgtattttcctgcataatcatatggttgacagtatgcattaagaaggtaagtaaaacaatgaagacaattttgatttaatatggtaatgcacaattccaactaacgtacattcaacagatcatgaaattgggttatt

SEQ ID NO: 216 - DNA, 159 bp, Homo sapiens
atgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatg

SEQ ID NO: 217 - DNA, 231 bp, Homo sapiens
acacagaagtatttggcaaagcccaacaccttcccccactggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgcctaccttaatgcctctgaaacaaccactctgagcccttctggaag

SEQ ID NO: 218 - DNA, 262 bp, Homo sapiens
gagtgtctcagaagtgtgctcctctggcctcagttctcctcttttggaacaacataaaacaaatttaattttctacgcctctggggatatctgctcagccaatggaaaatctgggttcaaccagcccctgccatttcttaagactttctgctccactcacaggatcctgagctgcacttacctgtgagagtcttcaaacttttaaaccttgccagtcaggacttttgctattgcaaatagaaaacccaactcaacctgctta

SEQ ID NO: 219 - DNA, 280 bp, Homo sapiens
tcatgtcagtgaagccatgtcaccatatcatatttttgaatgaactctgagtcagttgaaatagggtaccatctaggtcagtttaagaagagtcagctcagagaaagcaagcataagggaaaatgtcacgtaaactagatcagggaacaaaatcctctccttgtggaaatatcccatgcagtttgttgatacaacttagtatcttattgcctaaaaaaaaatttcttatcattgtttcaaaaaagcaaaatcatggaaaatttttgttgtccaggcaaat

SEQ ID NO: 220 - DNA, 189 bp, Homo sapiens
gagagttcaactaagaaaggtcacatatgtgaaagcccaaggacactgtttgatatacagcaggtattcaatcagtgttatttgaaaccaaatctgaatttgaagtttgaattttctgagttggaatgaatttttttctagctgagggaaactgtatttttctttccccaaagaggaatgtaatgtaaa

SEQ ID NO: 221 - DNA, 285 bp, Homo sapiens
ttctcagtcagtctatgccagtctataaggttttgtggggatgctcacagtaagggcactgaaaggagcaagtttgtcaggtcactgcaatgagtaggccatgctttcagttcttcctatctggtcagctgccttctggcactccagctggatttacgagccaaaattatggtttgaggtgttgtaattggctaagtctctggacaaaaattacccaaaataatctgaaactttactggccaaagtgggactcctttaaaattccaaaacttgccagtctacaca

SEQ ID NO: 222 - DNA, 259 bp, Homo sapiens
  misc_feature (85)..(85): n is a, c, g, or t
aacatgagtgcactttactaatcctcatggcacagtggctcacgcctgtaatcccagcacttgggaggacaatgtgggtggatcncgaggtcaggagttcgagaacagcctggccaacatggtgaaaccccgtctccactaaaaatacaaaaattagccaggcatggtggcgtacacttgtaattccagctactcaagaggctgaggcaggaggattgcttgaaccctgaaggcagaggttacagagccaagatagcgc

SEQ ID NO: 223 - DNA, 299 bp, Homo sapiens
atactatcccgttggtatttcccagtggctgaaaacctgattttctgctgcacgtggcatctgattacctgtggtcactgaacacacgaataacttggatagcaaatcctgagacaatggaaaaccattaactttacttcattggcttataaccttgttgttattgaaacagcacttctgtttttgagtttgttttagctaaaaagaaggaatacacacaggaataatgaccccaaaaatgcttagataaggcccctatacacaggacctgacatttagctcaatgatgcgtttgtaag

SEQ ID NO: 224 - DNA, 268 bp, Homo sapiens
aataaagtcgcttactcagtcacccagctacatgatttgtggctctggaagttcagctactaggactcttccaacttcctacaggtgtcacgaaagcacaagaccacacacttacacacagttctgatgaaagagggctaaaaggaaaatttcagtaaaaaggtcaacaaggctttctaaaaaaactgtaaagataaataaatgatgtgtcgcagtaattctcaccaattaggaagcatcttagaaaagcattggatttcctctagtt

SEQ ID NO: 225 - DNA, 191 bp, Homo sapiens
ctggccactcgcaagaccttttatttgaaaaccagccaagctttattcacgacacactttttcccttcactctcccacttctgtggtcaactccctgcagaactcccaaactgccgttcttttcgatagctcacgatggtgtatgagtgtcaatcatctgacccttcttggagtctcatatttcgtggaac

SEQ ID NO: 226 - DNA, 243 bp, Homo sapiens
gaaggtagacactgaactatgctggtagcttttccattggtatatttgtctccagattagagaggcgtgtcaaggcctgaaggagcccatgtggttggataaaatcaagaaaaggtgaatgagcacggttacccccaagtggaggggtttgtacaagacatgcgcctcatcttccagaaccacagggctcttacaagtataaggattttggcaaatgggacttagactggaggtgaaatttga

SEQ ID NO: 227 - DNA, 241 bp, Homo sapiens
ataaccctgttacaaagctgtgttgttgcttcttgtgaaggccatgatattttgtttttccccaattaattgctattgtgttattttactacttctctctgtattttttcttgcattgacattatagacattgaggacctcatccaaacaatttaaaaatgagtgtgaagggggaacaagtcaaaatatttttaaaagatcttcaaaagtaatgcctctgtctagcatgccaacaagaatg

SEQ ID NO: 228 - DNA, 201 bp, Homo sapiens
caggaaggtagcttgaagtcaggaatttaagacagtctgggcaacatagtgagacccccatctctataaatgctttttaaaagtagcagggcatggtggcatgtgcctgcaatctcagctacttggatgggtgagttgggagcgtcgcttgagcccaggagttctgagctgcagtgagctgtggttgcactactgagctgt

SEQ ID NO: 229 - DNA, 254 bp, Homo sapiens
agtgcctcatgcctttgggaggccaaggcaggaaggtagcttgaagtcaggaatttaagacagtctgggcaacatagtgagacccccatctctataaatgctttttaaaagtagcagggcatggtggcatgtgcctgcaatctcagctacttggatgggtgagttgggagcgtcgcttgagcccaggagttctgagctgcagtgagctgtggttgcactactgagctgtgattgcactcaaggctgggccacag

SEQ ID NO: 230 - DNA, 285 bp, Homo sapiens
gtgcagaactacaccaactggagcaccagcccctacttcctggagcatggcatcccccccagctgctgcatgaacgaaactgattgtaatccccaggatctacacaatctgactgtggccgccaccaaagttaaccagaagggttgttatgatctggtaactagtttcatggagactaacatgggaatcatcgctggagtggcgtttggaatcgcattctcccagttaattggcatgctgctggcctgctgtctgtcccggttcatcacggccaatcagtatgag